(19) United States
(12) Patent Application Publication
(10) Pub. No.: US 2010/0323376 A1
(43) Pub. Date: Dec. 23, 2010

(54) Method for Measuring Lipoprotein-Specific Apolipoproteins

(75) Inventor: John Contois, Windham, ME (US)

Correspondence Address: DRINKER BIDDLE & REATH;ATTN: INTELLECTUAL PROPERTY GROUP, ONE LOGAN SQUARE, SUITE 2000, PHILADELPHIA, PA, 19103-6996, US

(73) Assignee: Maine Standards Company, LLC

(21) Appl. No.: 12/817,026

(22) Filed: Jun. 16, 2010

Related U.S. Application Data
(60) Provisional application No. 61/187,806, filed on Jun. 17, 2009.

(51) Int. Cl. G01N 33/53; G01N 33/566

(52) U.S. Cl. 435/7.92; 435/7.1; 436/501

(57) ABSTRACT

The present invention is directed to methods of measuring the concentration of lipoprotein particles and/or lipoprotein-specific apolipoproteins in a biological fluid using an immunoassay, without the need of preliminary physical separation of the various types of lipoprotein particles present in the biological fluid.

### CROSS-REFERENCE TO RELATED APPLICATION

This application claims priority under 35 U.S.C. §119(e) to U.S. Provisional Application No. 61/187,806, filed Jun. 17, 2009, which application is incorporated by reference herein in its entirety.

### BACKGROUND OF THE INVENTION

Lipoproteins are intricate protein-lipid structures, in which the lipids or their derivatives may be covalently or non-covalently bound to the proteins. Among the various lipoproteins that play a key role in metabolic processes are blood lipoproteins, which enable fats (lipids) to be carried in the blood stream. Blood lipoproteins may be classified based on their size and lipid content, with lipoproteins with higher lipid content tending to be larger and less dense.

Chylomicrons (density<0.95 g/ml, and diameter of 100-1000 nm) carry triacylglycerol (fat) from the intestines to the liver, skeletal muscle and adipose tissue. Very low density lipoproteins (VLDL) (density of 0.95-1.006 g/ml, and diameter of 30-80 nm) carry newly synthesized triacylglycerol from the liver to adipose tissue. Intermediate density lipoproteins (IDL) (density of 1.006-1.019 g/ml, and diameter of 25-50 nm) are intermediate in size between VLDL and LDL. Low density lipoproteins (LDL) (density of 1.019-1.063 g/ml, and diameter of 18-28 nm) carry cholesterol from the liver to cells of the body. High density lipoproteins (HDL) (density>1.063 g/ml, and diameter of 5-15 nm) collect cholesterol from the tissues of the body, and bring it back to the liver.

Lipid levels are intrinsically associated with coronary disease or coronary heart disease (CHD). CHD represents the failure of coronary circulation to supply adequate circulation to cardiac muscle and surrounding tissue. CHD is often associated with coronary artery disease, where lipid-containing plaques accumulate within the walls of the coronary arteries that supply the myocardium (the heart muscle) with oxygen and nutrients. Among the various causes for development of CHD is dyslipidemia, a condition associated with a deviation in standard amounts of lipid in the blood of a subject. Such deviation may include one of the following occurrences, or any combination thereof: increase in total cholesterol (TC) level, increase in triglyceride (TG) level, decrease in high-density lipoprotein (HDL) cholesterol level, increase in low-density lipoprotein (LDL) cholesterol level, or presence of elevated LDL particles. Medical experts generally believe that management of CHD may be achieved by close monitoring and correction of lipid levels (especially LDL levels).

HDL is sometimes referred to as the “good cholesterol” lipoprotein, and higher levels of circulating HDL are generally associated with lower risk for CHD.

On the other hand, LDL cholesterol is widely accepted as a major risk factor for coronary heart disease (CHD), and is sometimes referred to as the “bad cholesterol” lipoprotein. Intervention trials with LDL-lowering therapies have been shown to reduce the incidence of future CHD events. LDL particles are heterogeneous in their composition of cholesterol, triglycerides, and phospholipids, and individuals with the same concentration of LDL particles may present very different LDL cholesterol levels. Conversely, individuals with the same LDL cholesterol levels may present very different concentration of LDL particles.

The concentration of plasma LDL cholesterol is a good predictor of CHD in a population study, but is frequently found to be a poor predictor of CHD when dealing with an individual patient. The main reason behind this discrepancy is that the LDL particles—and not the cholesterol contained within their core—interact with the arterial wall to promote atherosclerosis (Tabas et al., 2007, Circulation 116:1832; Rudd et al., In: “Textbook Of Cardiovascular Medicine”, 2^{nd} ed.; R. J. Topal, Ed., Lippincott, Williams, and Wilkins, Philadelphia, 2002, pp. 2-12). The higher the number of LDL particles in circulation, the higher the rate of development and progression of atherosclerosis. Therefore, it follows that a better predictor of future CHD events in a patient is the concentration of circulating LDL particles in the patient. Table 1 summarizes prospective epidemiologic studies of LDL particles (measured by NMR) and CHD.

TABLE 1
Prospective studies of LDL particle concentration as a risk factor for CHD
(adapted from Contois et al., 2009, Clin. Chem. 55: 407-419)

| | | | | Follow- | | # | # |
|---|---|---|---|---|---|---|---|
| Study | Design | Subjects | Endpoint | up | Fasting | Cases | Controls |
| Cardiovascular | Nested | M, W | MI, A | 5 y | unknown | 434 | 500 |
| Health Study - | CC | age ≧ 65 | | | | | |
| Kuller 2002 | | | | | | | |
| Women's Health | Nested | W | CHDD, | 3 y | unknown | 130 | 130 |
| Study - | CC | | MI, S | | | | |
| Blake 2002 | | | | | | | |
| Framingham | Cohort | M, W | MI, A, CI, | 14.8 y | Yes | 531 | 2,535 |
| Heart Study - | | | CHDD, S, | | | | |
| Cromwell 2007 | | | TIA, IC, | | | | |
| | | | CHF | | | | |
| EPIC-Norfolk | Nested | M, W | CHD, CHDD | 6 y | No | 1,003 | 1,885 |
| Prospective | CC | | | | | | |
| Population Study - | | | | | | | |
| E1 Harchaoui | | | | | | | |
| 2007 | | | | | | | |
| Women's Health | Cohort | W | CHD, | 11 y | unknown | 1,015 | 26,658 |
| Study - | | | CHDD | | | | |
| Mora 2007 | | | | | | | |
| PLAC-I Trial - | Cohort | M, W | ΔMLD | 3 y | unknown | 241 | — |
| Rosenson 2002 | | ↑LDL | | | | | |
| VA-HIT - | Nested | M < 74 y | MI, CHDD | 5.1 y | Yes | 364 | 697 |
| Otvos 2006 | CC | | | | | | |

Abbreviations:
MI, fatal or nonfatal myocardial infarction;
A, angina;
CHD, coronary heart disease;
CHDD, coronary heart disease death;
M, men;
W, women;
CC, case-control;
y, years;
A, angina;
S, stroke;
ΔMLD, change in minimum lumen diameter by angiography;
CI, coronary insufficiency;
IC, intermittent claudication;
CHF, congestive heart failure;
LDL-P, low-density lipoprotein particle concentration.

Due to the heterogeneous nature of LDL, the concentration of LDL particles (LDL-P) cannot be accurately estimated from the level of LDL cholesterol (Otvos, 1999, Clin. Cardiol. 22:1121). Various analytical methods have been used to measure LDL-P. As an example, prospective epidemiological studies using nuclear magnetic resonance (NMR) spectroscopy (Otvos, U.S. Pat. Nos. 5,343,389 and 6,576,471) to measure LDL-P showed significantly stronger associations of CHD outcomes with LDL particle concentration than with LDL cholesterol or other lipid and lipoprotein parameters (Table 2; Contois et al., 2009, Clin. Chem. 55:407-419), most notably, in the VAHIT Trial (Otvos et al., 2006, Circulation 113:1556), the Women's Health Study (Mora et al., Scientific Sessions of the American Heart Association, Orlando, Fla., November, 2007), and the Framingham Heart Study (Cromwell et al., 2007, J. Clin. Lipidol. 1:583). In the Multi-Ethnic Study of Atherosclerosis (MESA), the concentration of LDL particles correlated well with preclinical atherosclerosis (carotid intima-media thickness), even in subjects with LDL cholesterol less than 100 mg/dL (Mora et al., 2007, Atherosclerosis 2007, 192:211). Unfortunately, use of NMR technology is expensive, experimentally complicated, and not yet amenable for routine use in the clinical laboratory. Therefore, there is need in the art to quantitate the number or concentration of LDL particles in an individual patient using a method that may be routinely performed in a clinical setting.

Apolipoproteins, the protein components of lipoproteins, serve three major functions: (a) modulate the activity of enzymes that metabolize lipoproteins; (b) maintain the structural integrity of lipoproteins, and (c) facilitate the uptake of lipoprotein by binding to specific cell surface receptors. Apolipoprotein B (apo B) is a large protein with two isoforms: apo B-100, which is synthesized in the hepatocytes; and apo B-48, a truncated protein that is also derived from the apo B-100 gene but synthesized in the small intestine (Elovson et al., 1988, J. Lipid Res. 29:1461). Apo B-48 is found on chylomicrons, while apo B-100 is found on VLDL, IDL, LDL, and lipoprotein(a) [Lp(a)]. In all these lipoproteins, there is one molecule of apo B per lipoprotein particle, and therefore measurement of apo B associated with a particular lipoprotein class indicates the particle number or concentration of that particular lipoprotein class (Sniderman et al., 1991, Atherosclerosis 89:109).

TABLE 2
Prospective studies of LDL particle concentration:
Comparison of relative risk with LDL cholesterol level
(adapted from Contois et al., 2009, Clin. Chem. 55: 407-419)

| | | | | Matching and/or |
|---|---|---|---|---|
| | | | | Adjustment |
| Study | Comparison | LDL-P | LDL-C | Variables |
| Blake | Q4 v. Q1 | 4.17 (1.96-8.87) | 2.06 (1.03-4.12) | Age, smoking, |
| 2002 | | | | treatment group |
| Kuller | Q4 v. Q1 | M: NS | M: NS | Age, race |
| 2002 | | W: 2.59 | W: 3.34 | |
| Rosenson | Above v. | 2.1 (0.7-5.8) | 1.4 (0.5-3.9) | Age, race, baseline |
| 2002 | below the | | | lumen diameter |
| | median | | | |
| E1Harchaoui | Q4 v. Q1 | 1.78 (1.34-2.37) | 1.22 (0.92-1.61) | Smoking, SBP, |
| 2006 | | | | LDL-C or LDL-P |
| Otvos | Baseline, | 1.20 (1.05-1.37) | 1.10 (0.97-1.25) | Treatment group, |
| 2006 | 1 SD | | | age, HTN, |
| | On-Trial, | 1.28 (1.12-1.47) | 1.08 (0.95-1.23) | smoking, BMI, |
| | 1 SD | | | diabetes |
| Cromwell | 1 SD | M: 1.24 | M: 1.06 | Age, SBP, DBP, |
| 2007 | | (1.10-1.39) | (0.94-1.20) | smoking, |
| | | W: 1.33 | W: 1.18 | medication use |
| | | (1.17-1.50) | (1.02-1.37) | |
| Mora | Q5 v. Q1 | 2.51 (1.91-3.30) | 1.74 (1.40-2.16) | |
| 2007 | | | | |

Abbreviations:
apo B, apolipoprotein B;
NS, not significant;
M, men;
W, women;
T, tertile;
Q, quartile or quintile;
SD, standard deviation;
TC, total cholesterol;
TG, triglycerides;
BP, blood pressure;
SBP, systolic blood pressure;
DBP, diastolic blood pressure;
LDL-C, low-density lipoprotein cholesterol;
LDL-P, low-density lipoprotein particle concentration;
BMI, body mass index;
HDL-C, high-density lipoprotein cholesterol;
HTN, hypertension;
HbA1c, hemoglobin A1c;
TIA, transient ischemic attack;
PVD, peripheral vascular disease.

In an attempt to quantitate LDL, researchers have disclosed techniques such as ultracentrifugation, electrophoresis or selective precipitation, to separate LDL from other lipoproteins. However, these techniques are generally cumbersome, and thus lack the ease of implementation required for routine assay. Gambert described a method for measuring LDL-apo B using electrophoresis to separate LDL from other lipoproteins and then using electro-immunodiffusion to measure apo B (U.S. Pat. No. 5,064,769). Anderson described a method for measuring LDL-apo B by precipitating VLDL and IDL with anti-apo CI antibody, and then measuring the remaining apo B in solution (presumably associated with LDL) by rocket electrophoresis (Anderson, 1998, J. Lipid Res. 29:377). Vrga et al. described the isolation of LDL using an immunoseparation kit from Genzyme Diagnostics (Cambridge, Mass.) (Vrga et al., 1997, Clin. Chem. 43:390). The method employed anti-apo A-I and anti-apo E antibodies complexed to polystyrene beads as a means to separate VLDL and HDL from LDL. A filtrate containing the isolated LDL was obtained by centrifugation, and apo B in the filtrate was measured by an immunoassay. Schriewer et al. determined LDL cholesterol and LDL-apo B concentrations following precipitation of VLDL in blood serum with phosphotunstic acid and magnesium chloride (Schriewer et al., 1984, J Clin. Chem. Clin. Biochem. 22: 35). Caulfield et al. described a method for measuring LDL particle concentration by ion mobility analysis (Caulfield et al., 2008, Clin. Chem. 54:1307). Koren et al. described a number of monoclonal antibodies, including one antibody directed against an apo B epitope that was reportedly recognized on LDL but not VLDL particles (US Patent Application No. 2004/0053321). Slater et al. also described a monoclonal antibody based assay for LDL-apo B using a monoclonal antibody that displayed relative specificity for apo B associated to LDL (Slater et al., 1985, Clin. Chem. 31:841). These antibodies, or similar monoclonal antibodies directed at LDL-apo B, have yet to be widely adopted.

In a different approach, some researchers have attempted to estimate LDL-P based on total apo B measurements. Clendenen et al. described the use of total apo B to estimate LDL particle concentration, which may then be used to determine the particle concentrations of LDL subfractions separated by gradient gel electrophoresis (US Patent Application No. 2007/0072302). The method assumed a 1:1 relationship between total apo B and LDL-P, which is only a rough approximation. Similarly, Baca and Warnick developed a mathematical formula that estimates LDL-apo B based on total apo B and triglyceride concentrations (Baca & Warnick, 2008, Clin. Chem. 54: 907), and Kulkarni described a similar mathematical equation to estimate apo B (U.S. Pat. No. 7,521,248).

In summary, there is great interest in expeditiously determining LDL particle concentrations in a blood sample as a means to routinely monitor lipid levels and estimate risk for future CHD events in the subject. Unfortunately, there remains need for an accurate method of specifically measuring LDL particle numbers or concentrations in biological samples, where such method may be implemented in standard chemistry laboratories without the need for cumbersome purification procedures or expensive analytical instrumentation. The present invention fulfills this need.

### SUMMARY OF THE INVENTION

In one aspect, the invention includes a method of measuring the concentration of lipoprotein particles in a biological fluid of a subject, wherein an apolipoprotein is structurally associated with said lipoprotein in the form of lipoprotein-apolipoprotein.

In one embodiment, the method comprises mixing the biological fluid with Reagent R1, wherein Reagent R1 comprises a surfactant, to generate Solution S1; Solution S1 is then incubated for a period of time t1 at a set temperature T1; Solution S1 is then mixed with Reagent R2, wherein Reagent R2 comprises an antibody against the apolipoprotein, to generate Solution S2. Solution S2 is then incubated for a period of time t2 at a set temperature T2, to generate an apolipoprotein antibody complex. A calibration curve is then generated for quantitating binding of the antibody to the apolipoprotein with an immunoassay, using at least one standard solution comprising a known concentration of the apolipoprotein. Thus, the antibody-bound apolipoprotein is quantitated in Solution S2 in the immunoassay using the calibration curve, and, therefore, the concentration of the lipoprotein-apolipoprotein in Solution S2 is assessed, and, therefore, the concentration of the lipoprotein-apolipoprotein in the biological fluid is determined, and, therefore, the concentration of the lipoprotein particles in the biological fluid is assessed.

In another embodiment, the method comprises mixing the biological fluid with Reagent R1 to generate Solution S1; Solution S1 is then incubated for a period of time t1 at a set temperature T1; Solution S1 is then mixed with Reagent R2, wherein Reagent R2 comprises an antibody against the apolipoprotein and a surfactant, to generate Solution S2. Solution S2 is then incubated for a period of time t2 at a set temperature T2, to generate an apolipoprotein antibody complex. A calibration curve is then generated for quantitating binding of the antibody to the apolipoprotein with an immunoassay, using at least one standard solution comprising a known concentration of the apolipoprotein. Thus, the antibody-bound apolipoprotein is quantitated in Solution S2 in the immunoassay using the calibration curve, and, therefore, the concentration of the lipoprotein-apolipoprotein in Solution S2 is assessed, and, therefore, the concentration of the lipoprotein-apolipoprotein in the biological fluid is determined, and, therefore, the concentration of the lipoprotein particles in the biological fluid is assessed.

In yet another embodiment, the method comprises mixing the biological fluid with Reagent R2, wherein Reagent R2 comprises an antibody against the apolipoprotein and a surfactant, to generate Solution S2. Solution S2 is then incubated for a period of time t2 at a set temperature T2, to generate an apolipoprotein antibody complex. A calibration curve is then generated for quantitating binding of the antibody to the apolipoprotein with an immunoassay, using at least one standard solution comprising a known concentration of the apolipoprotein. Thus, the antibody-bound apolipoprotein is quantitated in Solution S2 in the immunoassay using the calibration curve, and, therefore, the concentration of the lipoprotein-apolipoprotein in Solution S2 is assessed, and, therefore, the concentration of the lipoprotein-apolipoprotein in the biological fluid is determined, and, therefore, the concentration of the lipoprotein particles in the biological fluid is assessed.

In one embodiment, the subject is human.

In one embodiment, the biological fluid is blood. In another embodiment, the biological fluid is serum. In yet another embodiment, the biological fluid is plasma.

In one embodiment, the lipoprotein is LDL and the apolipoprotein is apo B. In another embodiment, the lipoprotein is HDL and the apolipoprotein is selected from the group consisting of apo A-I, apo A-II, apo A-IV, apo A-V, apo C-I, apo C-II, apo C-III, apo C-IV, and apo E.

In one embodiment, the surfactant is an ionic surfactant. In another embodiment, the surfactant is a non-ionic surfactant. In yet another embodiment, the non-ionic surfactant is selected from the group consisting of a POE polymer, POP polymer, POE-POP block copolymer, PEG polymer, Brij® surfactant, Igepal® surfactant, Tween® surfactant and Triton® surfactant.

In one embodiment, the surfactant in Solution S1 varies in concentration from about 0.001% to about 10%. In another embodiment, the surfactant in Solution S1 varies in concentration from about 0.005% to about 1%.

In one embodiment, the surfactant in Solution S2 varies in concentration from about 0.001% to about 10%. In another embodiment, the surfactant in Solution S2 varies in concentration from about 0.005% to about 1%.

In one embodiment, Reagent R1 further comprises a PEG polymer. In another embodiment, the PEG polymer varies in concentration in Solution S1 from about 0.1% to about 10%. In yet another embodiment, the PEG polymer varies in concentration in Solution S1 from about 1% to about 8%.

In one embodiment, Reagent R2 further comprises a PEG polymer. In another embodiment, the PEG polymer varies in concentration in Solution S2 from about 0.1% to about 10%. In yet another embodiment, the PEG polymer varies in concentration in Solution S2 from about 1% to about 8%.

In one embodiment, Solution S2 further comprises a PEG polymer. In another embodiment, the PEG polymer varies in concentration in Solution S2 from about 0.1% to about 10%. In yet another embodiment, the PEG polymer varies in concentration in Solution S1 from about 1% to about 8%.

In one embodiment, Reagent R1 optionally further comprises one or more components selected from the group consisting of PEG, dextran sulfate, α-cyclodextrin sulfate, EDTA, an azide salt, and a divalent cation.

In one embodiment, Reagent R2 optionally further comprises one or more components selected from the group consisting of PEG, dextran sulfate, α-cyclodextrin sulfate, EDTA, an azide salt, and a divalent cation.

In one embodiment, Solution S2 optionally further comprises one or more components selected from the group consisting of PEG, dextran sulfate, α-cyclodextrin sulfate, EDTA, an azide salt, and a divalent cation.

In one embodiment, Solution S1 comprises surfactant Pluronic® F-68 (0.5%), PEG 4000 (2.5%), and PBS buffer, pH 7.4. In another embodiment, Solution S1 comprises surfactant Brij® 700 (0.005%), PEG 8000 (3%), and PBS buffer, pH 7.4. In yet another embodiment, Solution S1 comprises surfactant Pluronic® F-127 (0.01%), PEG 8000 (5%), and PBS buffer, pH 7.4.

In one embodiment, Solution S2 comprises surfactant Pluronic® F-68 (0.5%), PEG 4000 (2.5%), and PBS buffer, pH 7.4. In another embodiment, Solution S2 comprises surfactant Brij® 700 (0.005%), PEG 8000 (3%), and PBS buffer, pH 7.4. In yet another embodiment, Solution S2 comprises surfactant Pluronic® F-127 (0.01%), PEG 8000 (5%), and PBS buffer, pH 7.4.

In one embodiment, the period of time t1 is about 5 minutes and the set temperature T1 is about 37° C.

In one embodiment, the period of time t2 is about 3 minutes and the set temperature T2 is about 37° C.

In one embodiment, the immunoassay is immunoturbidimetry or immunonephelometry. In another embodiment, the immunoassay is an ELISA assay. In one embodiment, at least one standard solution has known concentration expressed in mg/dL units. In another embodiment, the concentration of the lipoprotein-apolipoprotein in the biological fluid is calculated in mg/dL units. In yet another embodiment, the concentration of the lipoprotein-apolipoprotein in the biological fluid is calculated in nmoles/L units. In yet another embodiment, the concentration of the lipoprotein particles in the biological fluid is calculated in nmoles/L units.

In another aspect, the invention includes a composition comprising a non-ionic surfactant selected from the group consisting of a POE polymer, a POP polymer, a POE-POP block copolymer, a PEG polymer, a Brij® surfactant, a Igepal® surfactant, a Tween® surfactant and a Triton® surfactant, wherein the concentration of said non-ionic surfactant varies from about 0.001% to about 10%; in a buffer with a pH value ranging from about 6.5 to about 8.5.

In one embodiment, the composition optionally further comprises a PEG polymer varying in concentration from about 0.1% to about 10%. In another embodiment, the composition optionally further comprises one or more components selected from the group consisting of PEG, dextran sulfate, α-cyclodextrin sulfate, EDTA, an azide salt and a divalent cation. In yet another embodiment, the composition further comprises a biological fluid from a subject. In yet another embodiment, the composition further comprises an antibody against an apolipoprotein.

In yet another aspect, the invention includes a kit for measuring the concentration of lipoprotein particles in a biological fluid of a subject, wherein an apolipoprotein is structurally associated with the lipoprotein in the form of lipoprotein-apolipoprotein. The kit comprises: (a) at least one composition comprising a known amount of said apolipoprotein; (b) a composition comprising a non-ionic surfactant selected from the group consisting of a POE polymer, a POP polymer, a POE-POP block copolymer, a PEG polymer, a Brij® surfactant, a Igepal® surfactant, a Tween® surfactant and a Triton® surfactant, wherein the concentration of the non-ionic surfactant varies from about 0.001% to about 10%; in a buffer with a pH value ranging from about 6.5 to about 8.5; this composition optionally further comprises a PEG polymer varying in concentration from 0.1% to about 10%; and optionally further comprising one or more components selected from the group consisting of PEG, dextran sulfate, α-cyclodextrin sulfate, EDTA, an azide salt and a divalent cation; and (c) a composition comprising an antibody against the apolipoprotein and optionally further comprising one or more components selected from the group consisting of a surfactant, PEG, dextran sulfate, α-cyclodextrin sulfate, EDTA, an azide salt and a divalent cation; wherein the kit further comprises an applicator and an instructional material for the use of the kit.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph representing the effect of surfactant Pluronic® P123, as a component of Reagent R1, in the reaction of anti-apo B antibody with VLDL and LDL particles, as measured by immunoturbidimetry. In all reactions, Reagent R1 contained 2.7% of PEG 4000. The extent of reaction for 0% Pluronic® P123 was normalized to 100%.

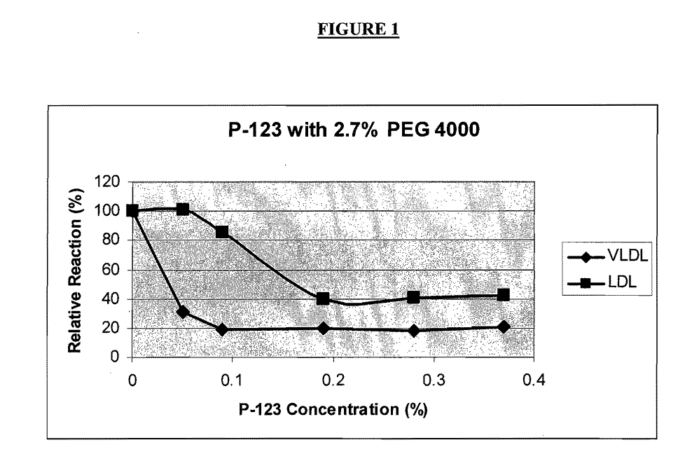

FIG. 2 is a graph representing the effect of surfactant Pluronic® L121, as a component of Reagent R1, in the reaction of anti-apo B antibody with VLDL and LDL particles, as measured by immunoturbidimetry. In all reactions, Reagent R1 contained 2.7% of PEG 4000. The extent of reaction for 0% Pluronic® P123 was normalized to 100%.

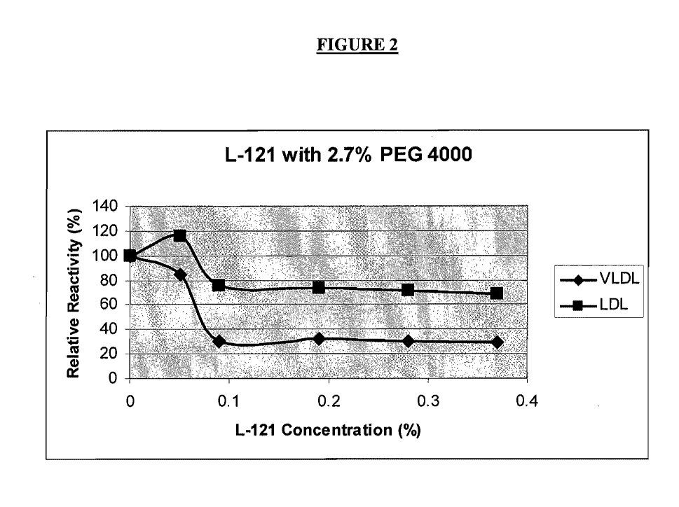

FIG. 3 is a graph representing the effect of PEG 4000, as a component of Reagent R1, in the reaction of anti-apo B antibody with VLDL and LDL particles, as measured by immunoturbidimetry. In all reactions, Reagent R1 contained 0.05% of Pluronic® P 123.

FIG. 4 is a graph representing the effect of PEG 600, as a component of Reagent R1, in the reaction of anti-apo B antibody with VLDL and LDL particles, as measured by immunoturbidimetry. In all reactions, Reagent R1 contained 0.8% of Pluronic® F68.

FIG. 5 is a graph representing the effect of PEG 4000, as a component of Reagent R1, in the reaction of anti-apo B antibody with VLDL and LDL particles, as measured by immunoturbidimetry. In all reactions, Reagent R1 contained 0.8% of Pluronic® F68.

FIG. 6 is a graph representing the effect of surfactant Pluronic® F68, as a component of Reagent R1, in the reaction of anti-apo B antibody with VLDL and LDL particles, as measured by immunoturbidimetry. In all reactions, Reagent R1 contained 3% PEG 4000.

FIG. 7 is a graph representing the effect of surfactant Pluronic® F68, as a component of Reagent R1, in the reaction of anti-apo B antibody with VLDL and LDL particles, as measured by immunoturbidimetry. In all reactions, Reagent R1 contained 3% PEG 600.

FIG. 8 is a graph and table illustrating the linear regression of observed and expected concentrations derived from dilution of concentrated LDL particles.

### DETAILED DESCRIPTION OF THE INVENTION

The invention relates to the discovery that the concentration of lipoprotein particles in solution may be expeditiously determined based on the quantitation of one or more apolipoproteins associated with the lipoprotein particles of interest, without the need for preliminary physical separation of the various types of lipoprotein particles present in the sample. The method thus advantageously avoids the use of costly and/or timely purification procedures or expensive analytical instrumentation.

The invention includes a method of measuring the concentration of LDL-apo B (e.g., apo B associated with LDL particles) in a biological fluid of a subject. The method comprises treating the biological fluid with a chemical reagent that “blocks” apo B associated with non-LDL particles (such as VLDL-apo B, i.e., apo B associated with VLDL particles) from binding to anti-apo B antibodies. Only apo B protein associated with LDL particles is then free to bind to anti-apo B antibody added to the treated sample. The method further comprises a method of quantitating the LDL-apo B concentration in the biological fluid and using that information to calculate the LDL particle concentration in the biological fluid.

The invention also includes a method of measuring the concentration of an apolipoprotein of choice bound to HDL in a biological fluid of a subject. The method comprises treating the biological fluid with a chemical reagent that “blocks” the apolipoprotein of choice (such as, but not limited to, apo A-I, apo A-II, apo A-IV, apo A-V, apo C-I, apo C-II, apo C-III, apo C-IV or apo E) that is associated with non-HDL particles (such as, but not limited to, VLDL), from binding to the corresponding anti-apolipoprotein antibodies (anti-apo A-I, anti-apo A-II, anti-apo A-IV, anti-apo A-V, anti-apo C-I, anti-apo C-II, anti-apo C-III, anti-apo C-IV or anti-apo E antibodies, respectively). The apolipoprotein of choice that is associated with HDL is then free to bind to the anti-apolipoprotein antibody added to the treated sample. The method further comprises a method of quantitating the concentration of apolipoprotein associated with HDL in the biological fluid and using that information to estimate the HDL particle concentration in the biological fluid.

The methods included in the invention may be used to monitor specific circulating levels of apolipoprotein and/or lipoprotein in the subject over time. This monitoring may be used to determine the subject's likelihood of developing coronary heart disease (CHD) and may be used as an integral part of a medical and/or lifestyle intervention, created for the individual with the objective of helping manage circulating level of the specific apolipoprotein and/or lipoprotein to ranges accepted as healthy or desirable by medical specialists.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, lipid chemistry, and nucleic acid chemistry and hybridization are those well known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used.

As used herein, the term “PBS” refers to phosphate-buffered saline, and the term “Tris” or “tris” refers to tris(hydroxymethyl)aminomethane base.

As used herein, the term “lipoproteinX-apolipoproteinY” refers to the apolipoproteinY that is structurally associated with lipoproteinX. For example, LDL-apo B refers to the apo B apolipoprotein that is structurally associated with LDL lipoprotein. Non-LDL-apo B refers to the apo B apolipoprotein that is structurally associated with lipoproteins that are not LDL. As a consequence, non-LDL-apo B corresponds to the total apo B apolipoprotein in the sample minus the apo B apolipoprotein that is structurally associated with LDL.

As used herein, the term “surfactant” refers to a wetting agent that lowers the surface tension of a liquid, allowing easier spreading, and lowers the interfacial tension between two liquids. A surfactant is usually an organic compound that is amphiphilic, meaning it contains both hydrophobic groups (“tails”) and hydrophilic groups (“heads”). Non-limiting examples of surfactants are polyoxyethylene (POE) polymers, polyoxypropylene (POP) polymers, POE-POP block copolymers, cyclodextrins, polyethyleneglycol (PEG), Brij® surfactants, Igepal® surfactants, Tween® surfactants, Triton® surfactants and dextran sulfate.

As used herein, the terms “peptide,” “polypeptide” and “protein” are used interchangeably, and refer to a compound comprised of amino acid residues covalently linked by peptide bonds. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof. A protein may be a receptor or a non-receptor.

As used herein, the term “fragment,” as applied to a protein or peptide, refers to a subsequence of a larger protein or peptide. A “fragment” of a protein or peptide may be at least about 10 amino acids in length; for example, at least about 50 amino acids in length; more preferably, at least about 100 amino acids in length; even more preferably, at least about 200 amino acids in length; particularly preferably, at least about 300 amino acids in length; and most preferably, at least about 400 amino acids in length.

As used herein, a “nucleic acid” refers to a polynucleotide and includes polyribonucleotides and polydeoxyribonucleotides.

As used herein, the term “homologous” refers to the subunit sequence similarity between two polymeric molecules, e.g., between two nucleic acid molecules, such as two DNA molecules or two RNA molecules, or between two polypeptide molecules. When a subunit position in both of the two molecules is occupied by the same monomeric subunit; e.g., if a position in each of two DNA molecules is occupied by adenine, they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. By way of example, the DNA sequences 3′ATTGCC5′ and 3′TATGGC5′ are 50% homologous. As used herein, “homology” is used synonymously with “identity.”

As used herein, the term “substantially the same” amino acid sequence is defined as a sequence with at least 70%, preferably at least about 80%, more preferably at least about 90%, even more preferably at least about 95%, and most preferably at least 99% homology to another amino acid sequence, as determined by the FASTA search method in accordance with Pearson & Lipman, Proc. Natl. Inst. Acad. Sci. USA 1988, 85:2444-2448.

As used herein, the term “isolated” means altered or removed from the natural state through the actions of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as a host cell for example.

As used herein, the term “antibody” refers to an immunoglobulin, whether natural or partly or wholly synthetically produced. The term also covers any polypeptide, protein or peptide having a binding domain that is, or is homologous to, an antibody binding domain. These may be isolated from natural sources, or may be partly or wholly synthetically produced. Examples of antibodies are intact immunoglobulin molecules, as well as fragments thereof, such as Fab, F(ab′)2, Fv fragments, and single chain variable fragments (scFv), which are capable of binding an epitopic determinant. Antibody fragments refer to antigen-binding immunoglobulin peptides that are at least about 5 to about 15 amino acids or more in length, and that retain some biological activity or immunological activity of an immunoglobulin. Antibody as used herein includes polyclonal and monoclonal antibodies, hybrid, single chain, and humanized antibodies, as well as Fab fragments, including the products of a Fab or other immunoglobulin expression library, and suitable derivatives.

As used herein, an antibody “specifically binds”, referring to an antibody binding to an apolipoprotein of choice, means that the antibody binds the apolipoprotein of choice, or subunit thereof, but does not bind to a biological molecule that is not the apolipoprotein of choice. Antibodies that specifically bind to an apolipoprotein of choice, or subunit thereof, do not substantially cross-react with biological molecules outside the apolipoprotein of choice.

As used herein, the term “monoclonal antibody” includes antibodies that display a single binding specificity and affinity for a particular epitope. These antibodies are mammalian-derived antibodies, including murine, human and humanized antibodies. As used herein, an “antibody heavy chain” refers to the larger of the two types of polypeptide chains present in all antibody molecules in their naturally occurring conformations. As used herein, an “antibody light chain” refers to the smaller of the two types of polypeptide chains present in all antibody molecules in their naturally occurring conformations.

As used herein, the term “anti-apolipoprotein-binding non-antibody molecule” should be construed to include organic molecules or peptides that are not antibodies and that bind to one or more of the apolipoproteins that are contemplated in the invention.

“Biologically active,” as used herein with respect to anti-apolipoprotein antibodies, fragments, derivatives, homologs or analogs, or anti-apolipoprotein-binding non-antibody molecules, means that the antibodies, fragments, derivatives, homologs or analogs, or anti-apolipoprotein-binding non-antibody molecules, have the ability to bind an apolipoprotein as described herein (e.g. anti-apo B antibody, as a non-limiting example). The term “inhibit,” as used herein, means to suppress or block an activity or function by at least about 10% relative to a control value. Preferably, the activity is suppressed or blocked by 50% compared to a control value, more preferably by 75%, and even more preferably by 95%.

“Derivative” includes any purposefully generated peptide that in its entirety, or in part, comprises an amino acid sequence substantially similar to a variable domain amino acid sequence of an antibody that binds one of the apolipoproteins contemplated in the invention. Derivatives of the antibodies of the present invention may be characterized by single or multiple amino acid substitutions, deletions, additions, or replacements. These derivatives may include: (a) derivatives in which one or more amino acid residues are substituted with conservative or non-conservative amino acids; (b) derivatives in which one or more amino acids are added; (c) derivatives in which one or more of the amino acids of the amino acid sequence used in the practice of the invention includes a substituent group; (d) derivatives in which amino acid sequences used in the practice of the invention or a portion thereof is fused to another peptide (e.g., serum albumin or protein transduction domain); (e) derivatives in which one or more nonstandard amino acid residues (e.g., those other than the 20 standard L-amino acids found in naturally occurring proteins) are incorporated or substituted into the amino acid sequences used in the practice of the invention; (f) derivatives in which one or more non-amino acid linking groups are incorporated into or replace a portion of the amino acids used in the practice of the invention; and (g) derivatives in which one or more amino acid is modified by glycosylation.

As used herein, the terms “subject”, “individual” or “patient” refer to the mammal, preferably human, whose biological fluid may be analyzed using the methods of the invention. As used herein, the term “biological fluid” includes any liquid material that is inside the body or organs of a subject. Biological fluids may be excreted or secreted from the body, or may be removed from the body. Biological fluids include, but are not limited to, amniotic fluid, blood, blood plasma, cerebrospinal fluid, interstitial fluid, lymph, breast milk, mucus (including nasal drainage and phlegm), pleural fluid, pus, sebum (skin oil), blood serum, and genital secretions.

As used herein, the term “applicator” refers to any device including, but not limited to, a hypodermic syringe, a pipette, an automatic sample probe and the like, for administering the compounds and compositions of the invention.

As used herein, the term “instructional material” includes a publication, a recording, a diagram, a product insert or any other medium of expression that may be used to communicate the usefulness of the composition and/or compound of the invention in the kit with respect to the methods of the invention. Optionally, or alternately, the instructional material may describe one or more methods related to the present invention, including as disclosed elsewhere herein.

The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Alternately, the instructional material may be obtained on the Internet in a format suitable for electronic file transmission to the user. For example, the instructional material is for use of a kit; instructions for use of the compound; or instructions for use of a formulation of the compound.

### Methods

The invention includes a method of measuring the concentration of an apolipoprotein associated with a lipoprotein in a biological fluid of a subject without the need to physically separate the given lipoprotein from other lipoproteins before performing the analysis.

In one embodiment of the invention, the apolipoprotein is apo B and the lipoprotein is LDL.

In another embodiment, the biological fluid is treated with a chemical reagent that “blocks” the apo B apolipoprotein associated with non-LDL particles (non-LDL-apo B) from binding to anti-apo B antibodies. Adding anti-apo B antibody to the treated sample leads to binding of the antibody only to the apo B protein associated with LDL particles (LDL-apo B). The invention includes a method of using this system to quantitate the LDL-apo B concentration in the biological fluid. The LDL-apo B concentration may then be used to calculate the LDL particle concentration in the biological fluid, using methods described herein.

In yet another embodiment, the biological fluid is treated with a chemical reagent that “blocks” the apo B protein associated with LDL particles (LDL-apo B) from binding to anti-apo B antibodies. Adding anti-apo B antibody to the treated sample leads to binding of the antibody only to the apo B protein associated with non-LDL particles (non-LDL-apo B). The invention includes a method of using this system to quantitate the non-LDL-apo B concentration in the biological fluid, which may be subtracted from the total apo B concentration to provide the LDL-apo B concentration in the biological fluid. The LDL-apo B concentration may then be used to calculate the LDL particle concentration in the biological fluid, using methods described herein.

In another embodiment of the invention, the lipoprotein is HDL, and the apolipoprotein is selected from the group consisting of apo A-I, apo A-II, apo A-IV, apo A-V, apo C-I, apo C-II, apo C-III, apo C-IV and apo E. The biological fluid is treated with a chemical reagent that “blocks” the apolipoprotein associated with non-HDL particles (non-HDL-apolipoprotein) from binding to anti-apolipoprotein antibodies. Adding anti-apolipoprotein antibody to the treated sample leads to binding of the antibody only to the apolipoprotein associated with HDL particles (HDL-apolipoprotein). The invention further relates to a method of quantitating the HDL-apolipoprotein concentration in the biological fluid. This information may then be used to estimate the HDL particle concentration in the biological fluid, based on the average number of apolipoprotein per HDL-apolipoprotein particle, using methods described herein.

The biological fluid under analysis may comprise any biological fluid isolated from a mammal, preferably a human. In one embodiment, the biological fluid is human blood. In another embodiment, the biological fluid is human serum. In yet another embodiment, the biological fluid is human plasma.

The invention includes a method of measuring the concentration of lipoprotein particles in a biological fluid of a subject, wherein an apolipoprotein is associated with the lipoprotein in the form of lipoprotein-apolipoprotein. In one embodiment, the method comprises mixing the biological fluid with Reagent R1, wherein Reagent R1 comprises a surfactant, to generate Solution S1; Solution S1 is incubated for a first period of time at a first set temperature; Solution S1 is mixed with Reagent R2, wherein Reagent R2 comprises an antibody against the apolipoprotein, to generate Solution S2. Solution S2 is then incubated for a period of time t2 at a set temperature T2, to generate an antibody-bound apolipoprotein. A calibration curve is then generated for quantitating binding of the antibody to the given apolipoprotein with an immunoassay of choice, using at least one standard solution comprising a known concentration of the apolipoprotein. Thus, the antibody-bound apolipoprotein in Solution S2 is quantified in the immunoassay of choice using the calibration curve, and the concentration in Solution S2 of the lipoprotein-apolipoprotein is assessed, and therefore, the concentration of the lipoprotein-apolipoprotein in the biological fluids determined; and therefore the concentration of the lipoprotein particles in the biological fluid of the subject is determined.

In another embodiment, the method comprises mixing the biological fluid with Reagent R1 to generate Solution S1; Solution S1 is incubated for a period of time t1 at a set temperature T1; Solution S1 is mixed with Reagent R2, wherein Reagent R2 comprises an antibody against the apolipoprotein and a surfactant, to generate Solution S2. Solution S2 is then incubated for a period of time t2 at a set temperature T2, to generate an antibody-bound apolipoprotein. A calibration curve is then generated for quantitating binding of the antibody to the given apolipoprotein with an immunoassay of choice, using at least one standard solution comprising a known concentration of the apolipoprotein. Thus, the antibody-bound apolipoprotein in Solution S2 is quantified in the immunoassay of choice using the calibration curve, and the concentration in Solution S2 of the lipoprotein-apolipoprotein is assessed, and therefore, the concentration of the lipoprotein-apolipoprotein in the biological fluids determined; and therefore the concentration of the lipoprotein particles in the biological fluid of the subject is determined.

In yet another embodiment, the method comprises mixing the biological fluid with Reagent R2, wherein Reagent R2 comprises an antibody against the apolipoprotein and a surfactant, to generate Solution S2. Solution S2 is then incubated for a period of time t2 at a set temperature T2, to generate an antibody-bound apolipoprotein. A calibration curve is then generated for quantitating binding of the antibody to the given apolipoprotein with an immunoassay of choice, using at least one standard solution comprising a known concentration of the apolipoprotein. Thus, the antibody-bound apolipoprotein in Solution S2 is quantified in the immunoassay of choice using the calibration curve, and the concentration in Solution S2 of the lipoprotein-apolipoprotein is assessed, and therefore, the concentration of the lipoprotein-apolipoprotein in the biological fluids determined; and therefore the concentration of the lipoprotein particles in the biological fluid of the subject is determined.

### Reagent R1

The biological fluid to be analyzed may be diluted with a water-based solution, referred to as Reagent R1. The resulting solution is referred to as Solution S1. The desirable degree of dilution of biological fluid with Reagent R1 to generate Solution S1 depends, among other factors, on the concentration of the apolipoprotein of interest in the biological fluid, the components of Reagent R1 and the sensitivity of the method used for detecting antibody binding to the apolipoprotein of interest. A person skilled in the art is able to identify, with a minimal amount of experimentation, an acceptable dilution value for the biological fluid with Reagent R1, so that the binding of apolipoprotein and anti-apolipoprotein antibody may be properly monitored.

Reagent R1 may be buffered using a buffer system that is known in the art not to interfere with immunoassays in general. In one embodiment, the buffer system used in Reagent R1 has an optimal buffering range of pH about 6.5 to pH about 8.5. In another embodiment, the buffer system used in Reagent R1 has an optimal buffering range of pH about 7.0 to pH about 8.0. In yet another embodiment, the buffer system used in Reagent R1 comprises PBS. In yet another embodiment, the buffer system used in Reagent R1 comprises Tris.

In one embodiment, Reagent R1 comprises a surfactant. In another embodiment, Reagent R1 comprises a non-ionic surfactant. In yet another embodiment, the concentration of the surfactant in Reagent R1 affords a final concentration of the surfactant in Solution S1 varying from 0.001 to about 10%. In one embodiment, the concentration of the surfactant in Reagent R1 affords a final concentration of the surfactant in Solution S1 varying from about 0.005 to about 1%.

A surfactant may be ionic or non-ionic in nature. An ionic surfactant is characterized by the presence of a net charge on its head group. If the charge is negative, the surfactant is more specifically called anionic. Anionic surfactants generally have head groups based on sulfate, sulfonate or carboxylate anions. If the charge is positive, the surfactant is called cationic. Cationic surfactants generally have head groups based on quaternary ammonium cations. In the case where a surfactant contains a head with two oppositely charged groups, it is termed zwitterionic or amphoteric.

Non-limiting examples of anionic surfactants are: perfluorooctanoate (PFOA or PFO); perfluorooctanesulfonate (PFOS); alkyl sulfate salts, such as sodium dodecyl sulfate (SDS) and ammonium lauryl sulfate; sodium lauryl sulfate, also known as sodium lauryl ether sulfate (SLES); alkyl benzene sulfonate; and soaps, or fatty acid salts.

Non-limiting examples of cationic surfactants are: alkyltrimethyl-ammonium salts, such as cetyl trimethylammonium bromide (CTAB), also known as hexadecyl trimethyl ammonium bromide; cetylpyridinium chloride (CPC); polyethoxylated tallow amine (POEA); benzalkonium chloride (BAC); and benzethonium chloride (BZT).

Non-limiting examples of zwitterionic or amphoteric surfactants are: dodecyl betaine; cocamidopropyl betaine; and coco ampho glycinate.

A non-ionic surfactant has no charge groups in its head. Non-limiting examples of non-ionic surfactants contemplated by the invention are POEs (polyoxyethylene polymers), POPs (polyoxypropylene polymers), POE-POP block copolymers, POE-POB block copolymers, Brij® surfactants, Igepal® surfactants, Tween® surfactants and Triton® surfactants.

Polyoxyethylene (POE), a synthetic polymer manufactured by polymerizing ethylene oxide, is also known as polyethylene glycol (PEG) or polyethylene oxide (PEO), and has the following structure:

HO—(CH₂—CH₂—O—)ₙ—H

Polyoxyethylene refers to oligomers and polymers of any molecular mass, and linear POEs are commercially available from 300 g/mole to 10,000,000 g/mole. For a PEG sample, the number cited after the “PEG” term represents the average molecular weight of the molecules in the sample. Non-limiting examples of PEG contemplated by the invention are PEG 200, PEG 300, PEG 400, PEG 550, PEG 600, PEG 1,000, PEG 1,500, PEG 2,000, PEG 3,000, PEG 3,350, PEG 4,000, PEG 4,400, PEG 6,000, PEG 6,200, PEG 8,000, PEG 10,000, PEG 12,000, PEG 20,000, PEG 35,000, PEG 40,000, PEG 55,000, PEG 108,000, PEG 218,000, PEG 246,000, PEG 463,000, and PEG 511,000.

Polyoxypropylene (POP), a synthetic polymer manufactured by polymerizing propylene oxide, is also known as polypropylene glycol (PPG) or polypropylene oxide (PPO), and has the following structure:

HO—(CH₂—CH(CH₃)—O—)ₙ—H

Polyoxybutylene (POB) is a synthetic polymer manufactured by polymerizing butylene oxide, and may have the following structure:

HO—(CH₂—CH(CH₂CH₃)—O—)ₙ—H

Non-ionic block copolymer surfactants may be made from butylene oxide, propylene oxide and ethylene oxide. The hydrophilic block is typically polyoxyethylene (POE), and the hydrophobic block is selected from polyoxypropylene (POP) and/or polyoxybutylene (POB). A typical POE-POP block copolymer, also known as poloxamer or Pluronic® (BASF, Florham Park, N.J.), has the following general structure (J. Am. Oil Chem. Soc. 1994, 71:777) with a central POP moiety and the two flanking hydrophilic POE groups:

HO(CH₂CH₂O)_{y}(CH₂CH(CH₃)O)ₓ(CH₂CH₂O)_{z}H or

HO(POE)_{y}(POP)ₓ(POE)_{z}H.

As evident from the general structure, POE-POP surfactants may vary widely in molecular weight and hydrophobicity, depending on the relative amounts of POE and POP. Also, the POE-POP surfactants may be chemically modified on the free hydroxyl groups, by esterification or alkylation, for example. A POP-POB block polymer has a similar structure to that of a POE-POP block polymer, wherein the POP residues are replaced with POB residues.

The composition of poloxamer or Pluronic® surfactant may be described by abbreviated nomenclature. Poloxamers are commonly named with the letter “P” (for poloxamer) followed by three digits: the first two digits, multiplied by 100, give the approximate molecular mass of the polyoxypropylene core, and the last digit, multiplied by 10, gives the percentage of polyoxyethylene content. For example, P-407 is a poloxamer with a polyoxypropylene molecular mass of 4,000 g/mol and a 70% polyoxyethylene content. For the Pluronic® tradename, the first letter in the code name refers to its physical form at room temperature: L=liquid, P=paste, F=flake (solid), and is followed by two or three digits. The first digit in a two-digit number or the first two digits in a three-digit number, multiplied by 300, indicates the approximate molecular weight of the hydrophobic block; and the last digit, multiplied by 10, gives the percentage of polyoxyethylene content. For example, L-61 is a Pluronic® surfactant with a polyoxypropylene molecular mass of 1,800 g/mol and a 10% polyoxyethylene content. Based on these conventions, poloxamer 181 (P-181) corresponds to Pluronic® L-61.

Non-limiting examples of Pluronics contemplated within the invention are: Pluronic® 10R5; Pluronic® 17R2; Pluronic® 17R4; Pluronic® 25R2; Pluronic® 25R4; Pluronic® 31R1; Pluronic® F-108; Pluronic® F-127; Pluronic® F-38; Pluronic® F-68; Pluronic® F-7 ; Pluronic® F-87; Pluronic® F-88; Pluronic® F-98; Pluronic® L-10; Pluronic® L-101; Pluronic® L-121; Pluronic® L-31; Pluronic® L-35; Pluronic® L-43; Pluronic® L-44; Pluronic® L-61; Pluronic® L-62; Pluronic® L-64; Pluronic® L-81; Pluronic® L-92; Pluronic® N-3; Pluronic® P-103; Pluronic® P-104; Pluronic® P-105; Pluronic® P-123; Pluronic® P-65; Pluronic® P-84; and Pluronic® P-85. Table 3 correlates Pluronic® and poloxamer nomenclatures, for a block polymer of formula HO(C₂H₄O)_{A}(C₃H₆O)_{B}(C₂H₄O)_{A}H.

TABLE 3
Correspondence of selected Pluronic ® and poloxamer surfactants.

| Pluronic ® | Poloxamer | “A” value | “B” value |
|---|---|---|---|
| L-44 | P-124 | 12 | 20 |
| F-68 | P-188 | 80 | 27 |
| F-87 | P-237 | 64 | 37 |
| F-108 | P-338 | 141 | 44 |
| F-127 | P-407 | 101 | 56 |

Brij® (ICI Americas, Wilmington, Del.) surfactant is a polyoxyethylene (POE) ether. Non-limiting examples of Brij® surfactants are: Brij® 30 (main component is tetraethylene glycol dodecyl ether, Mₙ˜362); Brij® 52 (main component is diethylene glycol hexadecyl ether, Mₙ˜330); Brij® 56 (main component is decaethylene glycol hexadecyl ether, Mₙ˜683); Brij® 700 (Mₙ˜4,670); Brij® 72 (main component is diethylene glycol octadecyl ether); Brij® 78 (main component is eicosaethylene glycol octadecyl ether); Brij® 92V (main component is diethylene glycol oleyl ether); Brij® 93 (Mₙ˜357); Brij® 97 (molecular formula C₁₈H₃₅—O—(CH₂CH₂)ₓH where x˜10, Mₙ˜709); Brij® 98 (Mₙ˜1,150); Brij® 010 (main component is decaethylene glycol oleyl ether); Brij® S10 (main component is decaethylene glycol octadecyl ether, Mₙ˜711); and Brij® 58 (Mₙ˜1124), where Mₙ is the number average molecular weight, e.g., the total weight of all the polymer molecules in the sample divided by the total number of polymer molecules in the sample.

Igepal® (Rhodia, Cranbury, N.J.) surfactants are homologous octylphenoxypoly(ethyleneoxy)ethanols, all derived from the same hydrophobic material (octylphenol) and varying lengths of hydrophilic tails (ethylene oxide). Their chemical structure is illustrated by the following formula:

C₈H₁₇-p-phenyl-O—(CH₂CH₂O)ₙH

wherein “n” denotes the number of moles of ethylene oxide per mole of octylphenol. Changes in the hydrophobic-hydrophilic balance, linked to the value of “n”, influence wetting detergency, emulsification, solubility or foam, and some applications may require the mixing of two or more of the products for a specific use. Non-limiting examples of Igepal® surfactants are: CA-210 (n=1.5, with 24% ethylene oxide); CA-420 (n=3, with 40% ethylene oxide); CA-520 (n=5, with 50% ethylene oxide); CA-620 (n=7, with 60% ethylene oxide); CA-630 (n=9, with 65% ethylene oxide); CA-720 (n=12, with 73% ethylene oxide); CA-887 (n=30, with 87% ethylene oxide); CA-890 (n=40, with 90% ethylene oxide); and CA-897 (n-40, with 90% ethylene oxide).

Tween® surfactants (ICI Americas, Wilmington, Del.) are polysorbates, defined as oily liquids derived from PEG-ylated sorbitan (a derivative of sorbitol) esterified with fatty acids. Surfactants that are esters of plain (non-PEG-ylated) sorbitan with fatty acids are usually referred to by the name Span®. Non-limiting examples of Tween® surfactants are Polysorbate 20 [Tween® 20 or polyoxyethylene (20) sorbitan monolaurate]; Polysorbate 40 [Tween® 40 or polyoxyethylene (20) sorbitan monopalmitate]; Polysorbate 60 [Tween® 60 or polyoxyethylene (20) sorbitan monostearate]; and Polysorbate 80 [Tween® 80 or polyoxyethylene (20) sorbitan monooleate]. The number following the “polysorbate” term is related to the type of fatty acid associated with the polyoxyethylene sorbitan part of the molecule—monolaurate is indicated by 20, monopalmitate is indicated by 40, monostearate by 60 and monooleate by 80. The same numbering is followed in their Span® equivalents (Span® 20, Span® 40, Span® 60 and Span® 80). The number 20 following the “polyoxyethylene” term refers to the total number of oxyethylene —(CH₂CH₂O)— groups found in the molecule.

As mentioned above, Tween® 20 (Polysorbate 20) is a polyoxyethylene derivative of sorbitan monolaurate, with the molecular formula C₅₈H₁₁₄O₂₆ and the molecular mass of 1,227.5 g/mole.

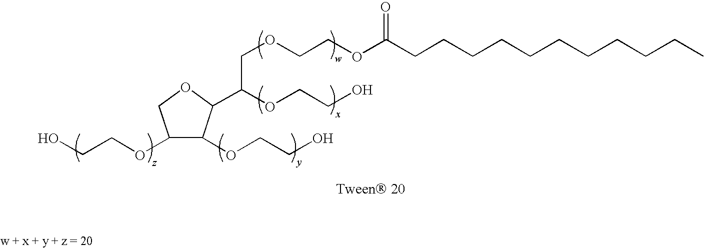

Tween® 80 (Polysorbate 80) is a polyoxyethylene derivative of sorbitan monooleate, with molecular formula of C₆₄H₁₂₄O₂₆ and molecular mass of 1,310 g/mole.

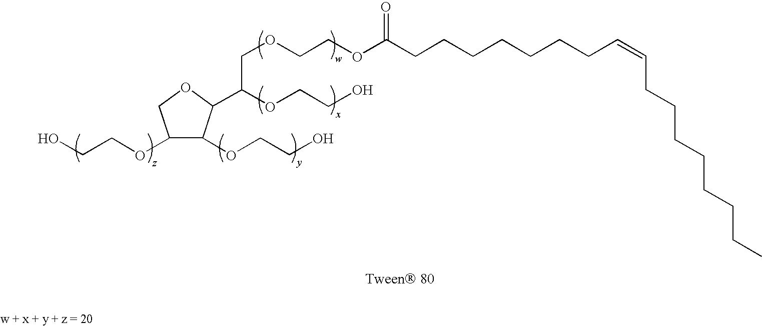

Triton® X-100 (Dow Chemical, Midland, Mich.) has the molecular formula C₁₄H₂₂O(C₂H₄O)ₙ and is a nonionic surfactant with a hydrophilic polyethylene oxide group (n˜9.5) and the lipophilic 4-(1,1,3,3-tetramethylbutyl)-phenyl group.

Reagent R1 may optionally comprise a polyethylene glycol polymer (PEG). PEGs are commercially available over a wide range of molecular weights from 300 g/mol to 10,000,000 g/mol. In one embodiment, the concentration of PEG in Reagent R1 affords a final concentration of PEG in Solution S1 varying from about 0.1% to about 10%. In another embodiment, the concentration of PEG in Reagent R1 affords a final concentration of PEG in Solution S1 varying from about 1% to about 8%.

Reagent R1 may optionally comprise one or more components selected from the group consisting of PEG, dextran sulfate, α-cyclodextrin sulfate, EDTA, an azide salt and a divalent cation. In one embodiment, the concentration of dextran sulfate in Reagent R1 affords a final concentration of dextran sulfate in Solution S1 varying from about 0.1 g/L to about 3.0 g/L. In another embodiment, the concentration of α-cyclodextrin sulfate in Reagent R1 affords a final concentration of α-cyclodextrin sulfate in Solution S1 varying from about 0.1 g/L to about 3.0 g/L. In yet another embodiment, the concentration of the azide salt in Reagent R1 affords a final concentration of azide in Solution S1 varying from about 0.01% to about 0.1%. In yet another embodiment, the azide salt is sodium azide. In yet another embodiment, the concentration of EDTA in Reagent R1 affords a final concentration of EDTA in Solution S1 varying from about 1 mM to about 50 mM. In yet another embodiment, the concentration of the divalent cation in Reagent R1 affords a final concentration of divalent cation in Solution S1 varying from about 1 mM to about 20 mM. In yet another embodiment, the divalent cation is magnesium. In yet another embodiment, the divalent cation salt is magnesium chloride.

### Solution S1

Upon mixing of Reagent R1 with the biological sample, the resulting Solution S1 may be incubated at a preset temperature T1 for a defined period of time t1 in order to allow equilibration between the various components of Solution S1. During this period of time, the surfactant molecules interact with the lipoproteins present, reaching equilibrium between unbound surfactant and lipoprotein-bound surfactant. One skilled in the art may easily determine the optimal time and temperature for this incubation by running parallel experiments with varying time and temperature values and determining which combinations of incubation temperature and incubation time provide a reproducible reading for the subsequent antibody binding measurement. One skilled in the art will also easily recognize that the optimal time and temperature for this incubation are dependent on the lipoprotein and surfactant used, as well as on the nature of the remaining components contained in Solution S1. In one embodiment of the invention, the incubation time t1 is about 5 minutes. In another embodiment, the incubation temperature T1 is about 37° C.

### Reagent R2

Solution S1 may then be contacted with Reagent R2, to generate Solution S2. Reagent R2 comprises an anti-apolipoprotein antibody. In one embodiment, the anti-apolipoprotein antibody is anti-apo B antibody. In another embodiment, the anti-apolipoprotein antibody is anti-apo A-I antibody. In yet another embodiment, the anti-apolipoprotein antibody is anti-apo A-II antibody. In yet another embodiment, the anti-apolipoprotein antibody is anti-apo A-IV antibody. In yet another embodiment, the anti-apolipoprotein antibody is anti-apo A-V antibody. In yet another embodiment, the anti-apolipoprotein antibody is anti-apo C-I antibody. In another embodiment, the anti-apolipoprotein antibody is anti-apo C-II antibody. In yet another embodiment, the anti-apolipoprotein antibody is anti-apo C-III antibody, In yet another embodiment, the anti-apolipoprotein antibody is anti-apo C-IV antibody. In yet another embodiment, the anti-apolipoprotein antibody is anti-apo E antibody.

Using conventional techniques, the skilled artisan may use the nucleotide and amino acid sequences of the apolipoprotein to prepare an antigenic peptide for use in generating corresponding anti-apolipoprotein antibody. The human apo B cDNA sequence (SEQ ID NO:1) is disclosed in Higuchi et al., 1988, Proc. Natl. Acad. Sci. USA 85 (6):1772-1776. Likewise, the cDNA sequences for human apo A-I (SEQ ID NO:2), apo A-II (SEQ ID NO:3), apo A-IV (SEQ ID NO:4), apo A-V (SEQ ID NO:5), apo C-I (SEQ ID NO:6), apo C-II (SEQ ID NO:7), apo C-III (SEQ ID NO:8), apo C-IV (SEQ ID NO:9) and apo E (SEQ ID NO:10) are known in the art.

Alternatively, the skilled artisan may utilize a commercially available antibody against the apolipoprotein (such as apo B). Anti-apo B and other anti-apolipoprotein antibodies are available from Midland Bioproducts (Boone, Iowa), US Biological (Swampscott, Mass.), Santa Cruz Biotechnology (Santa Cruz, Calif.), Millipore (Billerica, Mass.), R&D Systems (Minneapolis, Minn.) and Rockland Immunochemicals (Gilbertsville, Pa.), among others. The skilled artisan may also obtain commercially available anti-apolipoprotein antibodies and modify them using conventional methods such as coupling to other antibodies, partial digestion, pegylation or covalent modification. Modified antibodies may then be used in the methods of the invention as described herein. Antibodies useful in the practice of the present invention may be polyclonal, monoclonal, synthetic or fragments of any of the above.

It will be appreciated that the anti-apolipoprotein antibody used in the invention may be monovalent, divalent or polyvalent in order to achieve apolipoprotein binding. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

The invention also includes functional equivalents of the antibodies described herein. Functional equivalents have binding characteristics comparable to those of the antibodies, and include, for example, hybrid and single chain antibodies, as well as fragments thereof. Methods of producing such functional equivalents are disclosed in PCT Application Nos. WO 1993/21319 and WO 1989/09622. Functional equivalents include polypeptides with amino acid sequences substantially the same as the amino acid sequence of the variable or hypervariable regions of the antibodies raised against apolipoproteins, according to the practice of the present invention.

Functional equivalents of the anti-apolipoprotein antibodies further include fragments of antibodies that have the same, or substantially the same, binding characteristics to those of the whole antibody. Such fragments may contain one or both Fab fragments or the F(ab′)2 fragment. Preferably the antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Preferred constant regions are gamma 1 (IgG1), gamma 2 (IgG2 and IgG), gamma 3 (IgG3) and gamma 4 (IgG4). The light chain constant region can be of the kappa or lambda type.

The monoclonal antibodies may be advantageously cleaved by proteolytic enzymes to generate fragments retaining the apolipoprotein binding site. For example, proteolytic treatment of IgG antibodies with papain at neutral pH generates two identical so-called “Fab” fragments, each containing one intact light chain disulfide-bonded to a fragment of the heavy chain (Fc). Each Fab fragment contains one antigen-combining site. The remaining portion of the IgG molecule is a dimer known as “Fc”. Similarly, pepsin cleavage at pH 4 results in the so-called F(ab′)2 fragment.

Single chain antibodies or Fv fragments are polypeptides that consist of the variable region of the heavy chain of the antibody linked to the variable region of the light chain, with or without an interconnecting linker. Thus, the Fv comprises an antibody combining site.

Hybrid antibodies may be employed. Hybrid antibodies have constant regions derived substantially or exclusively from human antibody constant regions and variable regions derived substantially or exclusively from the sequence of the variable region of a monoclonal antibody from each stable hybridoma.

Methods for preparation of fragments of antibodies are known to those skilled in the art. See, Goding, “Monoclonal Antibodies Principles and Practice”, Academic Press (1983), p. 119-123. Fragments of the monoclonal antibodies containing the antigen binding site, such as Fab and F(ab′)₂ fragments, may be preferred in therapeutic applications, owing to their reduced immunogenicity. Such fragments are less immunogenic than the intact antibody, which contains the immunogenic Fc portion. Hence, as used herein, the term “antibody” includes intact antibody molecules and fragments thereof that retain antigen binding ability.

When the antibody used in the practice of the invention is a polyclonal antibody (IgG), the antibody is generated by inoculating a suitable animal with the apolipoprotein or a fragment thereof. Antibodies produced in the inoculated animal that specifically bind the apolipoprotein are then isolated from fluid obtained from the animal. Anti-apolipoprotein antibodies may be generated in this manner in several non-human mammals such as, but not limited to, goat, sheep, horse, rabbit, and donkey. Methods for generating polyclonal antibodies are well known in the art and are described, for example in Harlow et al. (In: Antibodies, A Laboratory Manual, 1988, Cold Spring Harbor, N.Y.). These methods are not repeated herein as they are commonly used in the art of antibody technology.

When the antibody used in the methods used in the practice of the invention is a monoclonal antibody, the antibody is generated using any well known monoclonal antibody preparation procedures such as those described, for example, in Harlow et al. (In: Antibodies, A Laboratory Manual, 1988, Cold Spring Harbor, N.Y.) and Tuszynski et al. (Blood 1988, 72: 109-115). Given that these methods are well known in the art, they are not replicated herein. Generally, monoclonal antibodies directed against a desired antigen are generated from mice immunized with the antigen using standard procedures as referenced herein. Monoclonal antibodies directed against full length or fragments of target structure may be prepared using the techniques described in Harlow et al. (In: Antibodies, A Laboratory Manual, 1988, Cold Spring Harbor, N.Y.).

The skilled artisan would further appreciate, based upon the disclosure provided herein, that the invention is not limited to the use of an antibody as the binding element for the apolipoprotein of interest. The invention also allows for the use of an anti-apolipoprotein-binding non-antibody molecule as the element that binds to one or more of the apolipoproteins that are contemplated in the invention. The anti-apolipoprotein-binding non-antibody molecule may bind to the apolipoprotein or a fragment of the apolipoprotein. Preferred anti-apolipoprotein-binding non-antibody molecules within the invention are aptamers. Aptamers are oligonucleic acid (also referred to as nucleic acid) molecules or peptide molecules that bind a specific target molecule. Nucleic acid aptamers are nucleic acid species that have been engineered through repeated rounds of in vitro selection or equivalently, SELEX (systematic evolution of ligands by exponential enrichment), to bind to various molecular targets such as small molecules, proteins, nucleic acids, and even cells, tissues and organisms. Aptamers are useful in biotechnological and therapeutic applications as they offer molecular recognition properties that rival that of the commonly used antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. See Ellington, A. D. & Szostak, J. W., 1990, “In vitro selection of RNA molecules that bind specific ligands”, Nature 346 (6287): 818-22; Bock et al., 1992, “Selection of single-stranded DNA molecules that bind and inhibit human thrombin”, Nature 355 (6360): 564-6; Drabovich et al., 2006, “Selection of smart aptamers by methods of kinetic capillary electrophoresis”, Anal Chem. 78 (9): 3171-8, all of which are incorporated herein by reference in their entireties. Aptamers useful within the invention may be selected and/or prepared according to the teachings of the art.

Reagent R2 may optionally further comprises a surfactant. In the case where Reagent R1 does not comprise a surfactant, Reagent R2 comprises a surfactant. In the case where the method does not use Reagent R1, Reagent R2 comprises a surfactant. In one embodiment, Reagent R2 comprises a non-ionic surfactant. In another embodiment, the concentration of the surfactant in Reagent R2 affords a final concentration of the surfactant in Solution S2 varying from 0.001 to about 10%. In yet another embodiment, the concentration of the surfactant in Reagent R2 affords a final concentration of the surfactant in Solution S2 varying from about 0.005 to about 1%. In yet another embodiment, the surfactant is selected from the group consisting of POEs (polyoxyethylene) polymer, POPs (polyoxypropylene) polymer, POE-POP block copolymer, PEG polymer, POE-POB block copolymer, Brij® surfactant, Igepal® surfactant, Tween® surfactant and Triton® surfactant.

Reagent R2 may optionally further comprise one or more components selected from the group consisting of a PEG polymer (“PEG”), dextran sulfate, α-cyclodextrin sulfate, EDTA, an azide salt and a divalent cation salt. The PEG may comprise any commercially available polyethylene glycol polymer ranging in molecular weight from 200 to 520,000. In one embodiment, the concentration of PEG in Reagent R2 varies from about 0.1% to about 10%. In another embodiment, the concentration of dextran sulfate in Reagent R2 varies from about 0.1 g/L to about 3.0 g/L. In another embodiment, the concentration of α-cyclodextrin sulfate in Reagent R2 varies from about 0.1 g/L to about 3.0 g/L. In yet another embodiment, the concentration of the azide salt in Reagent R2 varies from about 0.01% to about 0.1%. In yet another embodiment, the azide salt is sodium azide. In yet another embodiment, the concentration of EDTA in Reagent R2 varies from about 1 mM to about 50 mM. In yet another embodiment, the concentration of the divalent cation in Reagent R2 varies from about 1 mM to about 20 mM. In yet another embodiment, the divalent cation is magnesium. In yet another embodiment, the divalent cation salt is magnesium chloride.

### Solution S2

Solution S2 may be incubated for a defined period of time t2 and at a defined temperature T2 before the interaction of the anti-apolipoprotein antibody with the apolipoprotein is measured. The incubation time should be selected to allow approximate equilibration of the antibody within the solution, without causing unwanted sample degradation. One skilled in the art may easily determine the optimal time and temperature for this incubation by running parallel experiments with varying time and temperature values, and determining which combinations of incubation temperature and incubation time will provide a reproducible reading for the antibody binding measurement. One skilled in the art may also easily recognize that the optimal time and temperature for this incubation are dependent on the lipoprotein and anti-apolipoprotein antibody used, as well as on the nature of the remaining components contained in the second analysis solution. In one embodiment of the invention, Solution 2 is incubated for about 3 minutes. In another embodiment of the invention, Solution 2 is incubated at about 37° C.

### Analysis and Quantitation

Solution S2 may then be analyzed for binding of the anti-apolipoprotein antibody with the apolipoprotein contained therein. Any appropriate immunoassay to measure such an interaction is useful in the invention. Immunoassays are based on specific binding of an antibody to its antigen (in this particular case, the apolipoprotein of interest that is available to interact with the antibody). Detecting the interaction of the antibody with the antigen may be achieved using a variety of methods, of which one of the most common is to label either the antigen or antibody, and monitor the change in environment of the label upon binding. The label may comprise an enzyme (wherein binding is monitored by enzyme immunoassay or EIA), colloidal gold (wherein binding is monitored by lateral flow assays), radioisotopes such as ¹²⁵I radioimmunoassay (wherein binding is monitored by radiometric methods), magnetic labels (wherein binding is monitored by magnetic immunoassay or MIA) or fluorescence. Other techniques include, but are not limited to, agglutination, nephelometry, turbidimetry and Western Blot. All of these methods are known to those of skill in the art. See e.g. Harlow et al., 1988, “Antibodies: A Laboratory Manual”, Cold Spring Harbor Laboratory Press, Cold Spring Harbor, N.Y.; Harlow et al., 1999, “Using Antibodies: A Laboratory Manual, Cold Spring Harbor Laboratory Press”, Cold Spring Harbor, N.Y.

Immunoassays may be divided into those that involve non-labelled reagents and those that involve labelled reagents. Immunoassays that involve labelled reagents are divided into homogenous immunoassays and heterogeneous immunoassays (the latter require an extra step to remove unbound antibody or antigen from the site, usually using a solid phase reagent).

Heterogeneous immunoassays may be competitive or non-competitive. In a competitive immunoassay, the antigen in the unknown sample competes with labeled antigen to bind with antibodies. The amount of labeled antigen bound to the antibody site is then measured. In this method, the response will be inversely proportional to the concentration of antigen in the unknown, since a large response indicates that there is little antigen in the unknown to compete with the labeled antigen. In noncompetitive immunoassays, also referred to as the “sandwich assay,” antigen in the unknown is bound to the antibody site, then labeled antibody is bound to the antigen. The amount of labeled antibody on the site is then measured. Unlike the competitive method, the results of the noncompetitive method are directly proportional to the concentration of the antigen, since the labeled antibody will not bind if the antigen is not present in the unknown sample.

In certain embodiments, the immunoassay is selected from the group consisting of immunoturbidimetry, immunonephelometry, an ELISA assay, radioimmunoassay, chemiluminescence immunoassay, immunofluorescence, immunoprecipitation, immunoelectrophoresis, and flow cytometry-based immunoassay.

One skilled in the art will recognize that optimization studies may be easily performed to determine which chemical reagents should be present in Reagent R1 and/or Reagent R2 to allow for selective binding of the anti-apolipoprotein antibody to the apolipoprotein associated with the lipoprotein of choice over the apolipoprotein associated with other lipoproteins. The optimization studies may involve at least two lipid sources. The first lipid source comprises the apolipoprotein associated with the lipoprotein of choice, and the second source comprises the apolipoprotein associated to a lipoprotein that is not the lipoprotein of choice. The two lipid sources are incubated with Reagents R1 and R2 of differing compositions, as previously described, wherein each composition of Reagent R1 or Reagent R2 comprises varying concentrations of chemical reagents that may modulate the ability of the apolipoprotein of interest to bind to the corresponding anti-apolipoprotein antibody. Non-limiting examples of such chemical reagents are surfactants, non-ionic surfactants, divalent cation salts, dextran salts, PEG, α-cyclodextrin salts, EDTA, and azide salts. Following incubations, an immunoassay is used to determine the degree of antibody binding for each sample, and this information is used to determine the compositions for Reagent R1 and Reagent R2 that favor the binding of the antibody to the apolipoprotein associated with the lipoprotein of choice over the apolipoprotein associated with other lipoproteins. This evaluation follows standard methodologies used in analytical sciences and should not require unwarranted experimentation from those skilled in the art.

In a non-limiting example, wherein the apolipoprotein of interest is apo B and the given lipoprotein is LDL, the optimization studies are performed to identify compositions of Reagent R1 and/or Reagent R2 that “block” the apo B protein associated with non-LDL particles (non-LDL-apo B) from binding to anti-apo B antibodies. In this example, adding anti-apo B antibody to the treated sample leads to binding of the antibody only to the apo B protein associated with LDL particles (LDL-apo B). Therefore, the concentration of apo B determined in the assay corresponds to the concentration of LDL-apo B.

In another non-limiting example, wherein the apolipoprotein of interest is apo B and the given lipoprotein is LDL, the optimization studies are performed to identify compositions of Reagent R1 and/or Reagent R2 that “block” the apo B protein associated with LDL particles (LDL-apo B) from binding to anti-apo B antibodies. In this example, adding anti-apo B antibody to the treated sample leads to binding of the antibody only to the apo B protein associated with non-LDL particles (non-LDL-apo B). Therefore, the concentration of apo B determined in the assay corresponds to the non-LDL-apo B concentration, and the LDL-apo B concentration may be derived from the subtraction of the non-LDL-apo B concentration from the total apo B concentration (which may determined by an assay where the apo B from all lipoprotein particles is free to interact with the anti-apo B antibody, or any other published or known method to determine the total apo B concentration in a sample).

In yet another non-limiting example, wherein lipoprotein is HDL and the apolipoprotein of interest is selected from the group consisting of apo A-I, apo A-II, apo A-IV, apo A-V, apo C-I, apo C-II, apo C-III, apo C-IV and apo E, the optimization studies are performed to identify compositions of Reagent R1 and/or Reagent R2 that “block” the apolipoprotein of interest associated with non-HDL particles (non-HDL-apo B) from binding to anti-apolipoprotein antibodies. In this example, adding anti-apolipoprotein antibody to the treated sample leads to binding of the antibody only to the apolipoprotein associated with HDL particles. Therefore, the concentration of apolipoprotein of interest determined in the assay corresponds to the HDL-apolipoprotein concentration.

The immunoassay used to detect the interaction of the antibody with the apolipoprotein of interest may also be used to quantitate the concentration of the apolipoprotein in the sample. In a typical procedure included in the invention, a series of standard solutions containing known concentrations of the apolipoprotein of interest are prepared and analyzed by an immunoassay. The readings obtained for each standard solution are used to create a calibration curve. The unknown sample is then analyzed by the same immunoassay and its reading is compared to the standard curve in order to obtain a corresponding concentration of the apolipoprotein of interest in the sample. This concentration may be used to calculate the actual concentration of the apolipoprotein of interest in the biological fluid, taking into account the diluitions that the biological sample was subjected to for the preparation of Solution S2.

Use of the calibration curve, as described above, allows the concentration of the apolipoprotein to be determined in the same units used to express the concentration of the standard solutions. In some instances, the standard solutions have their component concentrations identified in mass/volume units (such as mg/dL units, for example). The concentration of the apolipoprotein of interest in the biological sample, determined as mg/dL from the calibration curve, may be converted to a concentration of moles/volume (such as nmol/L) based on the molecular weight of the apolipoprotein of interest. In the case where the apolipoprotein of interest is apo B, the molecular weight used for calculations is about 512,000 Daltons.

The concentration of apolipoprotein determined in the sample may be used to calculate the concentration of corresponding lipoprotein, depending on how many apolipoprotein molecules on average are contained in the lipoprotein. When the apolipoprotein of interest is apo B and the lipoprotein is LDL, there is one molecule of apo B per LDL molecule, and therefore the concentration of LDL-apo B (in molar-related units, such as nmole/L) is identical to that of LDL (in molar-related units, such as nmole/L). When there is an average of “N” apolipoprotein molecules of interest per lipoprotein, the concentration of lipoprotein (in molar-related units, such as nmole/L) is 1/N of the value of the concentration of apolipoprotein (in molar-related units, such as nmole/L).

As evident to those skilled in the art, the present invention encompasses variations that are included within the scope. As a non-limiting example, VLDL and HDL lipoprotein particles share a number of apolipoproteins, including apolipoprotein A-I (apo A-I), apolipoprotein A-II (apo A-II), apolipoprotein A-IV (apo A-IV), apolipoprotein A-V (apo A-V), apolipoprotein C-I (apo C-I), apolipoprotein C-II (apo C-II), apolipoprotein C-III (apo C-III), apolipoprotein C-IV (apo C-IV), and apolipoprotein E (apo E). The compositions of Reagent R1 and/or Reagent R2 that “block” VLDL-apo B from binding to anti-apo B antibodies may also “block” other VLDL-apolipoproteins (wherein the apolipoprotein is selected from the group consisting of apo A-I, apo A-II, apo A-IV, apo A-V, apo C-I, apo C-II, apo C-III, apo C-IV and apo E) from binding to the corresponding anti-apolipoprotein antibodies. In this case, such compositions of Reagent R1 and/or Reagent R2 may be used to promote selective binding of such anti-apolipoprotein antibodies to HDL-apolipoprotein, and this may facilitate determination of HDL-specific apolipoprotein concentrations directly.

The skilled artisan would appreciate, based on the present disclosure, that a composition of the invention may be modified based on the needs of a particular application. By way of a non-limiting example, a particular surfactant may be excluded from a particular analyte solution if the surfactant has the potential to interfere with the assay or with one or more other components of the analyte solution. Similarly, the skilled artisan will know, based on the present disclosure, that a method of the invention may be modified based on the needs of a particular application. By way of a non-limiting example, the immunoassay may utilize an endpoint reaction, wherein the concentration of apolipoprotein-antibody complex is measured at equilibrium. By the way of another non-limiting example, the immunoassay may measure the rate of formation of the apolipoprotein-antibody complex over a period of time or at one or more time points.

The skilled artisan would further appreciate, based upon the disclosures provided herein, that the invention is not limited to any particular instrument, but rather the invention encompasses a wide plethora of instruments as are known in the art or to be developed in the future. That is, such instruments for assessing the presence and/or level of a known constituent of interest in a sample include, but are not limited to, multi-channel chemistry analyzers and nephelometers such as, for instance, the Hitachi, Integra and Cobas analyzers from Roche Diagnostics (Indianapolis, Ind.), the Synchron, UniCel and AU systems from Beckman Coulter (Fullerton, Calif.), the Dimension, BN, IMS and Advia systems from Siemens Healthcare Diagnostics (Deerfield, Ind.), the Aeroset and Architect systems from Abbott Diagnostics (Abbott Park, Ill.), the Vitros system from Ortho Clinical Diagnostics (Rochester, N.Y.), and the Polychem from Polymedco (Cortlandt Manor, N.Y.). Thus, the skilled artisan would understand, based upon the disclosure provided herein, that the invention is not limited in any way to any particular instrument, either known or to be developed. Such instruments, including serum analyzers, hand-held devices, single test devices, and the like, are well-known in the art and are not discussed further herein.

As will be understood by one of skill in the art, when armed with the disclosure set forth herein, a set of reference apolipoproteins or equivalents (also referred to as “calibration samples”) may be used to create a calibration curve for a certain method and/or instrument. By way of a non-limiting example, the set of reference apolipoproteins or equivalents may be used in a two-point calibration assay. In another embodiment of the invention, the set of reference apolipoproteins or equivalents may be used in a five- or six point calibration assay. In one aspect, the set of reference apolipoproteins or equivalents may include as many or as few reference points as determined to be necessary to establish a valid and accurate reference curve.

Numerous calibration schemes may be used in the clinical laboratory. Older methods, often manually performed, employ several concentration levels throughout the assay range and typically plot the instrumental response versus concentration or use linear regression to calculate patient analyte values. These methods may still be used. However, with the increasing use and availability of computer technology, methods now often use one or two calibrator points to achieve the same results. Quite often, the one or two set point method incorporates a saline or distilled water blank as an additional set point, this latter function being dictated by the instrument or reagent manufacturer. For non-linear chemistries, the traditional approach provides five or six levels of calibrator, usually set in a non-linear fashion dictated by the mathematical model used in the final calculation of patient result. A more recent trend for non-linear chemistries is to use one calibrator containing the highest concentration of analyte measured in the assay. Using this method, the analytical system is then directed to perform the necessary dilutions of this high concentration value to generate the predetermined calibration set points on the fly when the system calibrates the analyte. A four- or five-parameter logit/log calibration curve is typically used for automated immunoassays.

Therefore, in an aspect of the present invention, there is provided a method that features the use of multiple calibrator points in order to generate a reference curve. In one embodiment, the method features the use of more than one point. In another embodiment, one of the multiple points is a zero point. In yet another embodiment, the zero point is not included as one of the multiple points, but may be included separately in a reference curve. In another embodiment, the method features the use of a single calibration point, as described in detail elsewhere herein. In yet another embodiment, the method features the use of a zero point in addition to a single calibration point.

By way of a series of non-limiting examples, the method of the invention may use a reference curve based on a single concentration for calibration, a reference curve based on a single concentration plus a zero concentration point for calibration, a reference curve based on at least two concentrations for calibration, or a reference curve based on at least two concentrations plus a zero concentration point for calibration. In yet another embodiment of the invention, the concentration of at least one calibration sample in a mixture containing at least two calibration samples is known.

### Kits

The invention includes various kits that comprise a set of apolipoprotein standards or equivalent (“calibration samples”), an applicator, and instructional materials that describe use of the kit to perform the methods of the invention. Although exemplary kits are described below, the contents of other useful kits will be apparent to the skilled artisan in light of the present disclosure. Each of these kits is included within the invention.

In one embodiment, the invention includes a kit for measuring the LDL-apo B concentration in a biological sample of a subject. The kit comprises reagents that allow for the determination of LDL-apo B in the presence of non-LDL-apo B. The kit further comprises an applicator and instructional material for the use of the kit.

In another embodiment, the invention includes a kit for measuring the non-LDL-apo B concentration in a biological sample of a subject. The kit comprises reagents that allow for the determination of non-LDL-apo B in the presence of LDL-apo B. The kit further comprises an applicator and instructional material for the use of the kit.

In yet another embodiment, the invention includes a kit for measuring the concentration of a specific apolipoprotein bound to HDL in a biological sample of a subject. The kit comprises reagents that allow for the determination of HDL-apolipoprotein in the presence of non-HDL-apolipoprotein. The kit further comprises an applicator and instructional material for the use of the kit.

The kit is used pursuant to the methods disclosed in the invention. In one embodiment, the kit may be used to determine the concentration of LDL-apo B in a biological sample. This is because, as more fully disclosed elsewhere herein, the data disclosed herein demonstrates that the reagents of the invention “block” binding of anti-apo B antibody to non-LDL-apo B. This allows for the direct detection and quantitation of LDL-apo B in the sample by the methods described herein.

The kit further comprises an applicator useful for administering the reagents for use in the relevant assay. The particular applicator included in the kit will depend on, e.g., the method used to assay a lipid, as well as the particular analyzer equipment used, and such applicators are well-known in the art and may include, among other things, a pipette, a syringe, a dropper bottle, and the like. Moreover, the kit comprises an instructional material for the use of the kit.

The kit includes a kit comprising Reagent R1 and/or Reagent R2 and/or various reagents used to prepare Reagent 1 and/or Reagent 2 as disclosed elsewhere herein, including, but not limited to, one or more surfactants, and one or more biological buffers. The composition is provided in an appropriate amount as set forth elsewhere herein.

Further, the kit includes a kit comprising at least one reference composition comprising a known value of a known constituent, which may be an apolipoprotein, lipoprotein, a derivative thereof or a fragment thereof Such kits may be used to create a calibration curve for quantitation of the apolipoprotein or lipoprotein. Thus, the invention encompasses a kit comprising at least one reference composition. While the invention is not limited to any particular set, certain combinations of reference compositions are exemplified elsewhere herein.

Further, the kit optionally comprises a lipoprotein, apolipoprotein or equivalent, including, but not limited to, total cholesterol, VLDL, LDL, HDL, apo A, apo B, Lp(a) and the like, and any combination thereof, as would be appreciated by one skilled in the art based upon the disclosure provided herein.

### Examples

The invention is now described with reference to the following Examples. These Examples are provided for the purpose of illustration only, and the invention is not limited to these Examples, but rather encompasses all variations that are evident as a result of the teachings provided herein.

### Materials

Anti-human apolipoprotein B polyclonal antibody (goat as host) was purchased from Midland Bioproducts, Boone, Iowa. Block copolymer surfactants Pluronic® P-123, L-121 and F-68, as well as all other surfactants, were purchased from Sigma-Aldrich (St. Louis, Mo.).

Immunoturbidimetric experiments were performed on a Roche Cobas Fara analyzer (Roche Diagnostic, Indianapolis, Ind.).

### Example 1

### Investigative Work Towards Development of a Method for Measuring LDL-Apo B Concentration or LDL Particle Concentration in a Biological Fluid

In order to identify conditions under which the anti-apo B antibody may bind preferentially to LDL-apo B over non-LDL-apo B, experiments were performed using lipoprotein fractions isolated from a serum pool. Ultracentrifugation was used to isolate chylomicrons, if present, and VLDL fractions (d<1.006 g/mL) from the LDL/HDL fraction (d>1.006 g/ml). Chylomicrons contribute relatively little apo B, especially in the fasting state, and HDL particles do not contain apo B. Therefore, these fractions above may be characterized as, respectively, “VLDL fraction” and “LDL fraction” for the purpose of apo B analysis.

The ability of each fraction to interact with anti-apo B antibody was evaluated using the Cobas Fara chemical analyzer. In a typical procedure, each fraction (6 μL) was mixed with 285 μL of Reagent R1. The composition of Reagent R1 was varied to investigate its influence on the interaction of apo B with the antibody. For this investigation, different block copolymer surfactants, other non-ionic detergents or salts were used to prepare Reagent R1. Among the block copolymer surfactants used in this investigation were three POE-POP surfactants: Pluronic® P-123, L-121 and F-68. Reagent R1 was prepared by diluting each surfactant to a final concentration of 0.05% (in the case of P-123 or L-121), or 0.4% (in the case of F-68), in PBS, pH 7.4.

The samples were loaded onto the analyzer, transferred to the analysis cuvettes, and incubated at about 37° C. for about 5 minutes. The resulting solution was treated with 95 μL of Reagent R2, prepared by diluting 1 part of anti-apo B antiserum with 7.5 parts of 50 mM Tris base, pH 8.0. The amount of apo B/anti-apo B antibody binding for each system was measured using immunoturbidimetry, based on absorbance relative to PBS.

The influence of various components in Reagent R1 on the apolipoprotein-antibody interaction is described below. As summarized in Tables 4 and 5, each of the 3 surfactants analyzed exhibited the ability of inhibiting anti-apo B antibody binding to VLDL-apo B over LDL-apo B. As examples of typical formulations, an R1 solution comprising 0.8% POE-POP and 5% PEG in PBS allowed complete inhibition of anti-apo B binding to VLDL particles, without altering binding of anti-apo B antibodies to LDL particles.

These experiments, the results of which are shown in Tables 4 and 5, indicate that reagents such as dextran sulfate, cyclodextrins and magnesium chloride, either alone or in combination, had little effect on the binding of anti-apo B antibody to VLDL-apo B or LDL-apo B. This result is in contrast to previously published reports (Sugiuchi, Clin. Chem. 1998, 44:522; U.S. Pat. Nos. 5,888,827 and 6,794,157) for direct LDL cholesterol measurement. Furthermore, these reagents did not influence the binding selectivity of anti-apo B antibody for VLDL-apo B or LDL-apo B in the presence of POE-POP block copolymers. This result differs from reports of assays for direct LDL cholesterol (LDL-C) and HDL cholesterol (HDL-C) measurements. In such reports, dextran sulfate, α-cyclodextrin sulfate and magnesium chloride were found to increase the selectivity that POE-POP block copolymers impart to the reactivity of enzymes used in cholesterol measurement (Sugiuchi, 1998, Clin. Chem. 44:522).

TABLE 4
Reactivity of apo B antibodies with VLDL and LDL fractions

| | VLDL RELATIVE | LDL RELATIVE |
|---|---|---|
| FORMULATION | REACTION (%) | REACTION (%) |
| PBS | 100 | 100 |
| P-123 | 31 | 107 |
| P-123, DS, MgCl₂ | 33 | 109 |
| P-123, αCDS, MgCl₂ | 35 | 117 |
| PEG 600 | 100 | 100 |
| PEG 600, P-123 | 34 | 108 |
| PEG 600, P-123, DS, MgCl₂ | 33 | 118 |
| PEG 600, P-123, αCDS, MgCl₂ | 33 | 110 |
| PBS | 100 | 100 |
| L-121 | 63 | 112 |
| L-121, DS, MgCl₂ | 77 | 108 |
| L-121, αCDS, MgCl₂ | 81 | 109 |
| PEG 600 | 100 | 100 |
| PEG 600, L-121 | 77 | 121 |
| PEG 600, L-121, DS, MgCl₂ | 73 | 108 |
| PEG 600, L-121, αCDS, MgCl₂ | 78 | 112 |
| PEG 4000 | 100 | 100 |
| PEG 4000, L-121 | 85 | 116 |
| PEG 4000, P-123 | 32 | 101 |
| PEG 4000, F-68 | 24 | 137 |
| PBS | 100 | 100 |
| αCDs | 100 | 115 |
| DS | 101 | 115 |
| MgCl₂ | 100 | 117 |
| αCDs, MgCl₂ | 107 | 132 |
| DS, MgCl₂ | 107 | 116 |
| Triton X-114, 0.1% | 44 | 62 |
| Triton X-114, 0.02% | 45 | 103 |
| Tween 20, 0.1% | 0 | 68 |
| Tween 20, 0.02% | 61 | 106 |

Final R1 concentrations for each reagent (if not indicated in Table 4): 0.05% P-123; 0.05% L-121; 0.4% F-68; 2.7% PEG 600; 2.7% PEG 4000; 0.15 g/L Dextran Sulfate (DS); 0.14% α-cyclodextrin sulfate (αCDs); 4.2 mM magnesium chloride (MgCl₂) in PBS, pH 7.4.

TABLE 5
Effect of F-68 surfactant on reactivity of anti-apo B antibodies with
VLDL and LDL fractions

| | VLDL RELATIVE | LDL RELATIVE |
|---|---|---|
| FORMULATION | REACTION (%) | REACTION (%) |
| PBS | 100 | 100 |
| 0.1% F-68 | 77 | 99 |
| 0.2% F-68 | 60 | 99 |
| 0.4% F-68 | 52 | 99 |
| 0.8% F-68 | 51 | 95 |
| 5% PEG 4000 | 100 | 100 |
| 5% PEG 4000, 0.1% F-68 | 67 | 102 |
| 5% PEG 4000, 0.2% F-68 | 54 | 102 |
| 5% PEG 4000, 0.4% F-68 | 34 | 98 |
| 5% PEG 4000, 0.8% F-68 | 27 | 96 |
| 5% PEG600 | 100 | 100 |
| 5% PEG600, 0.1% F-68 | 74 | 103 |
| 5% PEG600, 0.2% F-68 | 58 | 104 |
| 5% PEG600, 0.4% F-68 | 42 | 111 |
| 5% PEG600, 0.8% F-68 | 36 | 85 |

The data presented on Tables 4 and 5 also suggest that the hydrophobicity of the detergent does not have a significant effect on the relative reactivity of various formulations. This observation differs from literature reports of methods to measure lipoprotein-specific cholesterol or triglycerides (Sugiuchi, 1998, Clin. Chem. 44:522, and Wieland, U.S. Pat. No. 6,991,913).

Polyethylene glycol is typically added to immunoassay reagents to accelerate the interaction of antigen and antibody (Ritchie, “The Foundations of Immunochemistry”, Chapter 5, In: “the Immunoassay Handbook”, 3^{rd} Edition, David Wild, ed., Elsevier, 2005, pp. 91-2).

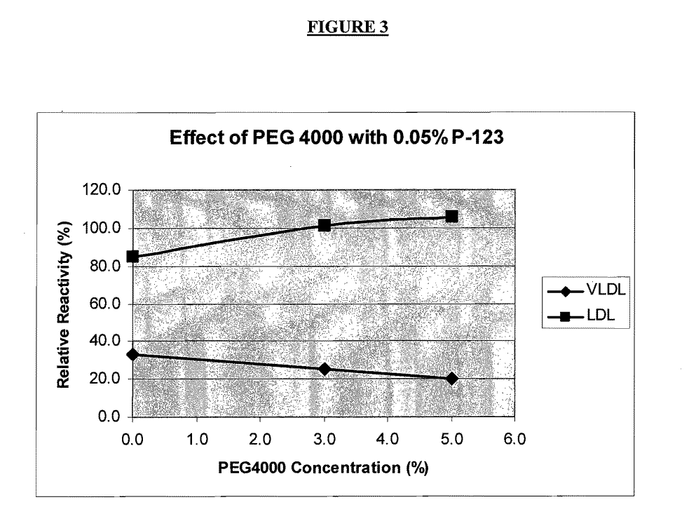

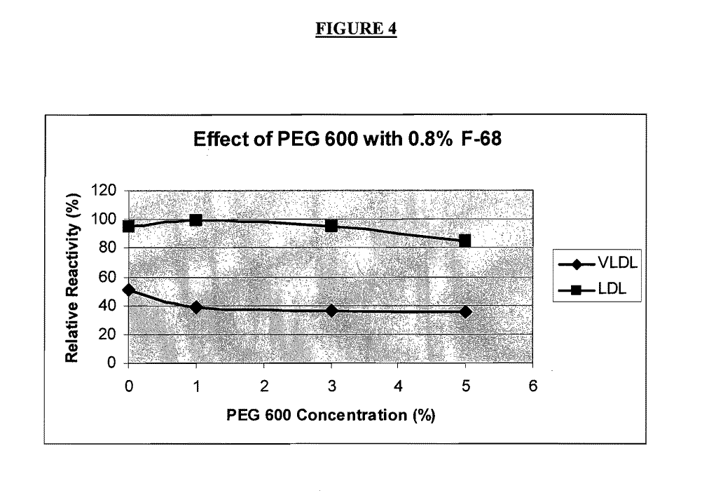

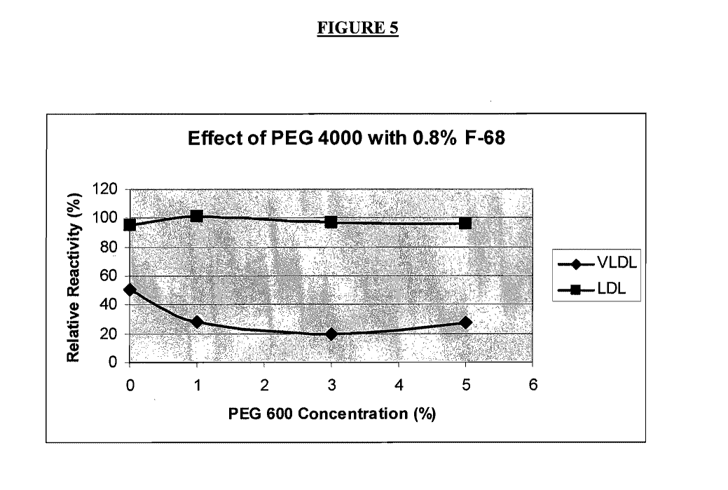

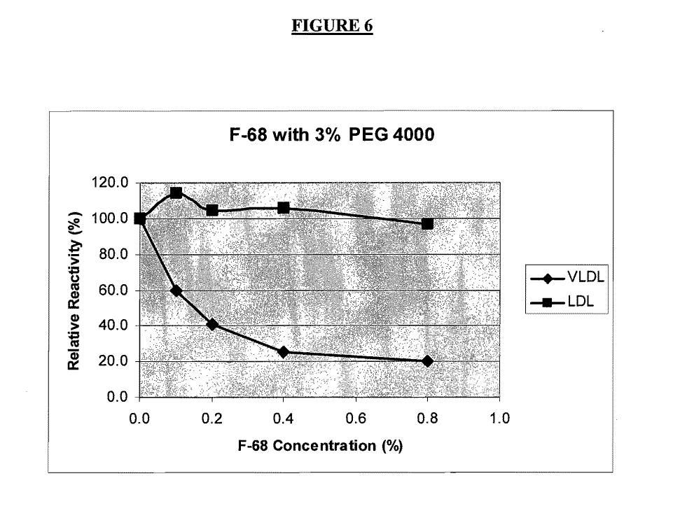

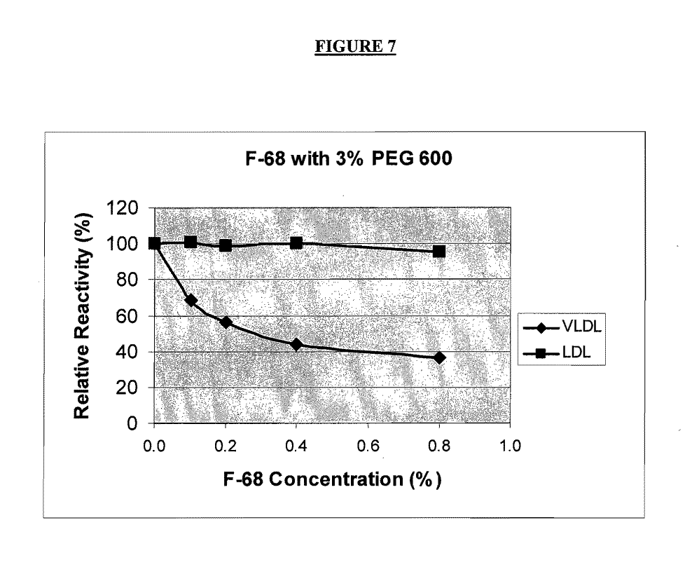

The data in Table 4 demonstrate that addition of PEG 600 or PEG 4000 does not influence the selectivity that P-123 or L-121 imparts on antigen-antibody interactions for LDL over VLDL, but in a subsequent experiment formulations with 0.05% P-123 and 0%, 3% or 5% PEG 4000 suggest otherwise (FIG. 3). The addition of PEG 4000 enhanced the recovery of LDL-apo B while inhibiting VLDL-apo B binding. Similarly, the addition of PEG 600 or PEG 4000 enhances the selectivity that F-68 imparts on the interaction of anti-apo B with LDL over VLDL (Table 4, and FIGS. 4-7).

Non-ionic detergents other than POE-POP block copolymers, such as Brij® 30, Brij® 700, Igepal® 630, and Tween® 20, were also found to inhibit reaction of anti-apo B antibody with VLDL, as shown in Table 6.

TABLE 6
Polyoxyethylene surfactants

| | VLDL RELATIVE |
|---|---|
| FORMULATION | REACTION (%) |
| PBS | 100 |
| 0.6% Brij ® 30 | 49.3 |
| 0.6% Brij ® 700 | 36.6 |
| 0.8% Igepal ® 630 | 78.9 |
| 0.4% Igepal ® 630 | 68.1 |
| 0.05% Tween ® 20 | 55.4 |
| PBS | 100 |
| 0.3% Brij ® 700 | 33.4 |
| 0.3% Brij ® 700, 3% PEG 4000 | 35.8 |
| 0.3% Brij ® 700, 0.05% Tween ® 20 | 33.8 |
| 0.3% Brij ® 700, 0.9% F-68 | 30.4 |

### Example 2

### Method for Measuring LDL-Apo B Concentration or LDL Particle Concentration in a Biological Fluid

Based on the optimization experiments described in Example 1, three Reagent R1 formulations were selected for further study.

### Reagent R1

Formulation 1: Pluronic® F-68 (0.5%); PEG 4000 (2.5%); PBS buffer, pH 7.4.
Formulation 2: Brij® 700 (0.005%); PEG 8000 (3%); PBS buffer, pH 7.4
Formulation 3: Pluronic® F-127 (0.01%); PEG 8000 (5%), PBS Buffer, pH 7.4

In a typical experiment, the assay was set up on a Cobas Fara analyzer using 6 μL sample of biological fluid, 285 μL of Reagent R1 (selected from one of the three formulations listed above), and 95 μL of Reagent R2. Reagent R2 consisted of a 1 part of apo B antiserum (Midland Bioproducts, Boone Iowa) diluted in 7.5 parts of 50 mM Tris base, pH 8.0. The biological sample was added to Reagent R1 and incubated at about 37° C. for about 5 minutes. After the “blank” absorbance measurement, Reagent R2 was added. About three minutes after Reagent R2 addition, the final reading was performed.

A four parameter log-logit calibration curve was prepared using c.f.a.s. lipid calibrators from Roche (Indianapolis, Ind.) using a serial dilution to give calibrator values of 232, 116, and 58 mg/dL, along with a normal saline zero calibrator. C.f.a.s. lipid calibrator is made from lyophilized human serum. Therefore, it is assumed to contain both VLDL and LDL apo B. This could in principle reduce the accuracy of measurement of LDL-apo B concentration, but the results of this experiment still demonstrate that the assays inhibit the reaction of VLDL-apo B with anti-apo B antibodies.

The different samples (VLDL fraction, LDL fraction and whole serum) were analyzed using the method described above and the results are shown in Table 7. Using the calibrator concentrations given above, the lipoprotein concentrations are provided in mg/dl units, which may be converted to nmole/l by multiplying by a factor of 19.5, based on a molecular weight of 512,000 for apo B (Knott et al., 1986, Nature 323:734-38; Chen et al., 1986, J. Biol. Chem. 261:12918-21; Law et al., 1986, Proc. Natl. Acad. Sci. USA 83:8142-46).

The results indicate that, for the three Reagents R1 used, the interaction of anti-apo B with VLDL was inhibited, while the interaction of anti-apo B with LDL was largely undisturbed.

TABLE 7
Quantitation of lipid fractions.

| | Reagent R1, | Reagent R1, | Reagent R1, |
|---|---|---|---|
| | Formulation 1 | Formulation 2 | Formulation 3 |
| Sample | concentration determined, nmole/L (mg/dL) | | |
| VLDL | 0 (0.0) | 8 (0.4) | 4 (0.2) |
| (d < 1.006 g/mL) | | | |
| LDL | 1539 (78.9) | 1835 (94.1) | 1712 (87.8) |
| (d > 1.006 g/mL) | | | |
| Serum | 1778 (91.2) | 1913 (98.1) | 1882 (96.5) |

### Example 3

### Assay Performance

Studies were performed to determine within-run and total precision, limit of detection, limit of quantitation, and linearity. Institutional review board (IRB) approval was obtained, and sera were obtained from volunteers who provided informed consent. Enough serum was available to separate VLDL and LDL by ultracentrifugation in 19 subjects. The LDL-apoB assay was calibrated with the WHO/IFCC SP3-08 reference material. This provided a reasonably accurate standard; however, the reference material is a stabilized serum pool that contains some VLDL.

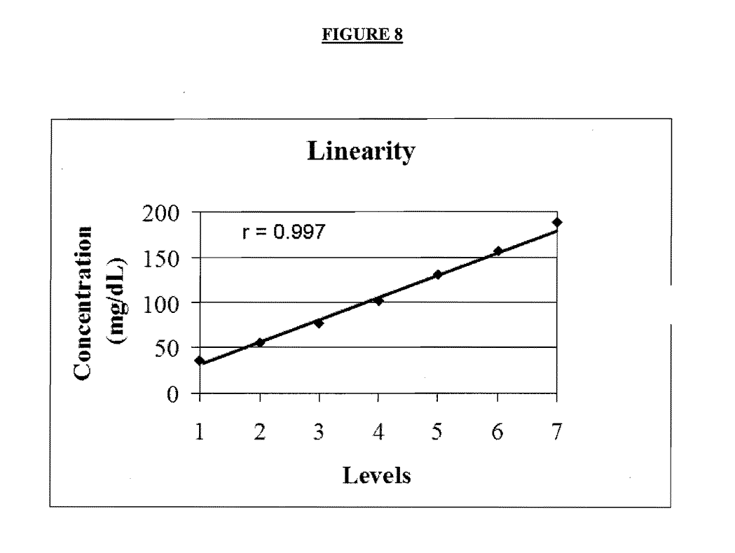

Data were collected on the Roche Cobas Fara analyzer as previously described, with the exception that 0.5 g/L dextran sulfate and 2 mM magnesium chloride were added to the R1 formulation, along with 0.6% Pluronic F-68. Within-run imprecision, assessed with three levels of QC material (52, 107, and 124 mg/dL), was 3.4%, 3.1, and 1.8%, respectively. Total imprecision, determined by measuring the same three levels of QC material in duplicate twice per day for 20 days, with four recalibration cycles, was 10.1%, 6.6%, and 6.0% for the low, medium, and high QC, respectively. The limit of detection, assessed with both normal saline and HDL supernatant (apoB particles removed from serum by precipitation with dextran sulfate-magnesium chloride) was 25 mg/dL (488 nmole/l). The limit of quantitation, defined as the concentration associated with 20% imprecision, was also <25 mg/dL. Linearity, assessed by linear regression of observed (y) and expected (x) concentrations derived from dilution of concentrated LDL, extended from about 30-183 mg/dL (FIG. 8). The measured values are listed in Table 8.

TABLE 8

| Level | Mean (mg/dL) | Expected (mg/dL) | Bias (mg/dL) |
|---|---|---|---|
| 1 | 35.9 | 30.0 | 5.9 |
| 2 | 55.3 | 55.5 | −0.2 |
| 3 | 76.9 | 80.9 | −4.1 |
| 4 | 101.6 | 106.4 | −4.8 |
| 5 | 130.6 | 131.8 | −1.3 |
| 6 | 155.9 | 157.3 | −1.4 |
| 7 | 188.5 | 182.7 | 5.8 |

Apo B in the VLDL fraction of 19 serum specimens, separated by ultracentrifugation, was also measured. Unlike initial studies using a serum pool, the addition of a small amount of dextran sulfate (0.5 g/L) and magnesium chloride (2.0 mM) improved the selectivity of the antiserum for LDL in most serum samples, especially those with elevated triglycerides. This was observed by Sugiuchi et al. for the direct LDL cholesterol assay, as well (Clin Chem 1998, 44:522-531) (Table 9). The assay was modified by increasing the sample: reagent volume ratio to increase low-end sensitivity and provide a limit of detection<1.0 mg/dL. Mean±SD concentration for VLDL-apo B measured with total apoB assay was 10.4±11.8. The mean VLDL-apo B concentration measured with the LDL-P assay was 1.1±3.5; indicating that the LDL-P reagent had successfully blocked binding of VLDL-apoB with the antiserum (Table 9). The exception was specimen 17, which had a triglyceride concentration of 632 mg/dL.

TABLE 9
VLDL-apoB measured with a total apoB assay and
with the LDL-apoB (LDL-P) assay

| | | Total | | LDL-P |
|---|---|---|---|---|
| ID | TRIG | APOB | LDL-P | (+DS/MgCl₂) |
| 1 | 141 | 4.6 | 1.4 | 0.1 |
| 2 | 160 | 4.6 | 2.1 | 0.3 |
| 3 | 88 | 4.4 | 0.1 | 0.0 |
| 4 | 208 | 14.1 | 2.3 | 1.0 |
| 5 | 50 | 9.1 | 0.1 | 0.1 |
| 6 | 121 | 19.6 | 1.1 | 0.1 |
| 7 | 158 | 1.9 | 1.2 | 0.2 |
| 8 | 174 | 10.5 | 2.0 | 0.2 |
| 9 | 111 | 12.3 | 0.7 | 0.0 |
| 10 | 173 | 9.6 | 2.2 | 0.4 |
| 11 | 111 | 1.8 | 0.4 | 0.2 |
| 12 | 282 | 10.1 | 5.7 | 1.3 |
| 13 | 177 | 7.7 | 1.5 | 0.3 |
| 14 | 70 | 2.1 | 0.1 | 0.0 |
| 15 | 25 | 0.8 | 0.1 | 0.0 |
| 16 | 160 | 10.3 | 1.4 | 0.2 |
| 17 | 632 | 54.6 | 33.0 | 15.5 |
| 18 | 256 | 13.2 | 2.6 | 0.8 |
| 19 | 151 | 6.9 | 0.9 | 0.1 |

| | | | |
|---|---|---|---|
| Mean ± SD | 10.4 ± 11.8 | 3.1 ± 7.4 | 1.1 ± 3.5 |

### INCORPORATION BY REFERENCE

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. claims

What is claimed:

1. A method of measuring the concentration of lipoprotein particles in a biological fluid of a subject, wherein an apolipoprotein is structurally associated with said lipoprotein in the form of lipoprotein-apolipoprotein, the method comprising the steps of:
incubating Solution S2, comprising said biological fluid, a surfactant, and an antibody against said apolipoprotein, for a period of time t2 at a set temperature T2, to generate an antibody-bound apolipoprotein;
generating a calibration curve for quantitating binding of said antibody to said apolipoprotein with an immunoassay, using at least one standard solution comprising a known concentration of said apolipoprotein;
quantitating said antibody-bound apolipoprotein in Solution S2 with said immunoassay using said calibration curve;
determining concentration of said lipoprotein-apolipoprotein in said Solution S2;
determining concentration of said lipoprotein-apolipoprotein in said biological fluid; and,
determining said concentration of said lipoprotein particles in said biological fluid.

2. The method of claim 1, wherein said subject is human.

3. The method of claim 1, wherein said biological fluid is selected from the group consisting of blood, serum and plasma.

4. The method of claim 1, wherein said Solution S2 is generated by a method comprising the steps of:
mixing said biological fluid with Reagent R1, wherein said Reagent R1 comprises a surfactant, to generate Solution S1;
incubating said Solution S1 for a period of time t1 at a set temperature T1; and,
mixing said Solution S1 with Reagent R2, wherein said Reagent R2 comprises said antibody against said apolipoprotein, to generate said Solution S2.

5. The method of claim 1, wherein said Solution S2 is generated by a method comprising the steps of:
mixing said biological fluid with Reagent R1, to generate Solution S1;
incubating said Solution S1 for a period of time t1 at a set temperature T1; and,
mixing said Solution S1 with Reagent R2, wherein said Reagent R2 comprises said surfactant and said antibody against said apolipoprotein, to generate said Solution S2.

6. The method of claim 1, wherein said Solution S2 is generated by a method comprising the step of:
mixing said biological fluid with Reagent R2, wherein said Reagent R2 comprises said surfactant and said antibody against said apolipoprotein, to generate said Solution S2.

7. The method of claim 1, wherein said lipoprotein is LDL and said apolipoprotein is apo B.

8. The method of claim 1, wherein said lipoprotein is HDL and said apolipoprotein is selected from the group consisting of apo A-I, apo A-II, apo A-IV, apo A-V, apo C-I, apo C-II, apo C-III, apo C-IV, and apo E.

9. The method of claim 1, wherein said surfactant is a non-ionic surfactant.

10. The method of claim 9, wherein said non-ionic surfactant is selected from the group consisting of a POE polymer, POP polymer, POE-POP block copolymer, PEG polymer, Brij® surfactant, Igepal® surfactant, Tween® surfactant and Triton® surfactant.

11. The method of claim 1, wherein said surfactant in said Solution S2 varies in concentration from about 0.001% to about 10%.

12. The method of claim 11, wherein said surfactant in said Solution S2 varies in concentration from about 0.005% to about 1%.

13. The method of claim 4, wherein said surfactant in said Solution S1 varies in concentration from about 0.001% to about 10%.

14. The method of claim 13, wherein said surfactant in said Solution S1 varies in concentration from about 0.005% to about 1%.

15. The method of claim 1, wherein said Solution S2 further comprises a PEG polymer.

16. The method of claim 15, wherein said PEG polymer varies in concentration in said Solution S2 from about 0.1% to about 10%.

17. The method of claim 16, wherein said PEG polymer varies in concentration in said Solution S2 from about 1% to about 8%.

18. The method of claim 4, wherein said Reagent R1 further comprises a PEG polymer.

19. The method of claim 18, wherein said PEG polymer varies in concentration in said Solution S1 from about 0.1% to about 10%.

20. The method of claim 19, wherein said PEG polymer varies in concentration in said Solution S1 from about 1% to about 8%.

21. The method of claim 1, wherein said Solution S2 optionally further comprises one or more components selected from the group consisting of PEG, dextran sulfate, α-cyclodextrin sulfate, EDTA, an azide salt, and a divalent cation.

22. The method of claim 4, wherein said Reagent R1 optionally further comprises one or more components selected from the group consisting of PEG, dextran sulfate, α-cyclodextrin sulfate, EDTA, an azide salt, and a divalent cation.

23. The method of claim 4, wherein said Reagent R2 optionally further comprises one or more components selected from the group consisting of PEG, dextran sulfate, α-cyclodextrin sulfate, EDTA, an azide salt, and a divalent cation.

24. The method of claim 1, wherein said Solution S2 comprises a composition selected from the group consisting of: (i) surfactant Pluronic® F-68 (0.5%), PEG 4000 (2.5%), and PBS buffer, pH 7.4; (ii) surfactant Brij® 700 (0.005%), PEG 8000 (3%), and PBS buffer, pH 7.4; and, (iii) Pluronic® F-127 (0.01%), PEG 8000 (5%), and PBS buffer, pH 7.4.

25. The method of claim 4, wherein said Solution S1 comprises a composition selected from the group consisting of: (i) surfactant Pluronic® F-68 (0.5%), PEG 4000 (2.5%), and PBS buffer, pH 7.4; (ii) surfactant Brij® 700 (0.005%), PEG 8000 (3%), and PBS buffer, pH 7.4; and, (iii) Pluronic® F-127 (0.01%), PEG 8000 (5%), and PBS buffer, pH 7.4.

26. The method of claim 1, wherein said period of time t2 is about 3 minutes and said set temperature T2 is about 37° C.

27. The method of claim 4, wherein said period of time t1 is about 5 minutes and said set temperature T1 is about 37° C.

28. The method of claim 1, wherein said immunoassay is immunoturbidimetry or immunonephelometry.

29. The method of claim 1, wherein said immunoassay is an ELISA assay.

30. The method of claim 1, wherein said at least one standard solution has said known concentration expressed in mg/dL units.

31. The method of claim 1, wherein said concentration of said lipoprotein-apolipoprotein in said biological fluid is calculated in mg/dL units.

32. The method of claim 1, wherein said concentration of said lipoprotein-apolipoprotein in said biological fluid is calculated in nmoles/L units.

33. The method of claim 1, wherein said concentration of said lipoprotein particles in said biological fluid is calculated in nmoles/L units.

34. A composition comprising a non-ionic surfactant selected from the group consisting of a POE polymer, a POP polymer, a POE-POP block copolymer, a PEG polymer, a Brij® surfactant, a Igepal® surfactant, a Tween® surfactant and a Triton® surfactant, wherein the concentration of said non-ionic surfactant varies from about 0.001% to about 10%; in a buffer with a pH value ranging from about 6.5 to about 8.5%; said composition optionally further comprising a PEG polymer varying in concentration from 0.1% to about 10%; and optionally further comprising one or more components selected from the group consisting of PEG, dextran sulfate, α-cyclodextrin sulfate, EDTA, an azide salt and a divalent cation.

35. The composition of claim 34, further comprising a biological fluid from a subject.

36. The composition of claim 34, further comprising an antibody against an apolipoprotein.

37. A kit for measuring the concentration of lipoprotein particles in a biological fluid of a subject, wherein an apolipoprotein is structurally associated with said lipoprotein in the form of lipoprotein-apolipoprotein, said kit comprising:
at least one composition comprising a known amount of said apolipoprotein,
a composition comprising a non-ionic surfactant selected from the group consisting of a POE polymer, a POP polymer, a POE-POP block copolymer, a PEG polymer, a Brij® surfactant, a Igepal® surfactant, a Tween® surfactant and a Triton® surfactant, wherein the concentration of said non-ionic surfactant varies from about 0.001% to about 10%; in a buffer with a pH value ranging from about 6.5 to about 8.5; said composition optionally further comprising a PEG polymer varying in concentration from about 0.1% to about 10%; and optionally further comprising one or more components selected from the group consisting of PEG, dextran sulfate, α-cyclodextrin sulfate, EDTA, an azide salt and a divalent cation;
a composition comprising an antibody against said apolipoprotein and optionally further comprising one or more components selected from the group consisting of PEG, dextran sulfate, α-cyclodextrin sulfate, EDTA, an azide salt and a divalent cation;
said kit further comprising an applicator and an instructional material for the use of said kit.


SEQUENCE LISTING (10 sequences)

SEQ ID NO: 1 - DNA, 42645 bp, Homo sapiens
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccggtgagtgcgcggccgctctgcgggcgcagagggagcgggagggagccggcggcacgaggttggccggggcagcctgggcctaggccagagggagggcagccacagggtccagggcgagtggggggattggaccagctggcggcccctgcaggctcaggatggggggcgcgggatggaggggctgaggagggggtctccggagcctgcctccctcctgaaaggtgaaacctgtgccggtggtccccctgtcgggccctagcacccgctgggaagacgtgggaagctcacagatttctttctcctgtcttacagaagaggaaatgctggaaaatgtcagcctggtctgtccaagtaaggcatctgcgcatggggcgtggaagggcgcccagccccgtgcactctcctacacccgggtccctgagggcctcccactctacagggctgagatggcatcgtggtgtgccttgctctgaccccaggaagcaagttccctgagcctctgcccacacccaagggatgccaactctcttctacctggccttctgttctgtcccaaaagttcagcctgggggcgggggagggaagggattgtctctccgctggcctgtgcacactttgaagaaacatcactgtcctgtttatcagtgactagtcattgattcgaagcatgtgagggtgaggaaatactgactttaacctttgtgaagaaatcgaacctccacccccttcctatttacctgacccctgggggttaaaggaactggcctccaagcgcgaccctgtgtgctggagccgcggggcggacttctgatggggcagcaccgccatctagtggccgtctgtcatcactgcagctggactcaggacccagatgttctttttcttcaattgttcagaaaattcctctcaactacagtggaaacctccagaaattcttttctaggagtttgttaagttagttacgcttaatgcttaatgaactttgccttaagtatttggtagtcttagagtcacggaattacggcgtgttcaagctaaaaaagcattagagatagtactatttgcgtaatgttgtcatctcttaatttgccagagggtctctcatgcagattttctgagccccattacttgacacttgtcactcccttccctgtgcctcagatgagatattcaagacatgccagccaatttaaacattagcctcagcaaaaacataatggagaagtcaaatctataaaggaaaattaagtataaagtcaattaaaaaataatttgagttgaattaccatttttaattctctatgccactgcccctctctgcccagaattggctgtccttgggagagctatttctgctatgtggctgacgtatttctccccacgttagaagatgcgacccgattcaagcacctccggaagtacacatacaactatgaggctgagagttccagtggagtccctgggactgctgattcaagaagtgccaccaggatcaactgcaaggtatggaggatgcaggcaggagggacctagagcccacagctttcccccagccctgttccagcgggcgcccaacacgcgaccttcccggagggtgtgtactgagcaaacgcagaacatcccagaactgttgtaatctgatcaaagcactgggactttgcctctgtttgtaagtcagccacattgctgagatgtggtctgcccccaccaaatttcgcaagtcagaagtattttcccgttaacttcccagatgcaataggaatccatgatctagattagcagcagtgtgggtctgtagatttcagcgtgagagaggcccagtaggtgagctatgggaggcaggcaactcggaatcgcactgtgaaatgcagtttttataatttaagtcaaacagaatctgttgctgaaaaatgaatggaaagaagaaaaaaatataaacatacagtttgttctaaaataaaactttgcttattattgagactggttgtactcatgttacatacatgtggagcagatctacaggctgctattggggtttgggtggggaagagaagtcaagctgagcagtcacctttttttagagagtaccgtagctcttgtatgtgctgtccaatatggtagacatgagccacattgggctatttaaatggaatgaaattaaaaattcatattcgttgtcacattagctgcatttcaactgctcaacagccaccctggctactggctcccatattgaacagcacacatgtacaacatttctataaagttatttgaatagtgctggataataagtaggaatccgttgaaactccagctatatgcaaagctctaaataggccctaatagatataaccagttttttgggtgacattaaggagacatttgctgtggaaacgaaggatggccctcttcctgctttctgtttttcttcttcactttcactcctagtctgcagcgcttctatttaaccacagctctttataattaaagtgagtaactttagaaccaataaaaggacatcctccttcccatgcctaggggcaaacttaagaaatgtgttacccgggagggggaaaacgtcagcaataggactaagtctaggttggtgcacagagaacccaggaggcatgttgataaggcatgtggtgttgaggcgcaggcagtggtgttcccagcaccattccctttggtgctctgattagagattaagccctgggcttcaggggccacctctcattcttgatagacaacctcaatgctctgctaccctgaattctcaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtaagtcatgttgtacatgagcacacgcatgtgtgtgtgtccgctgaggtatgaacttgtgtgtttgcaccaggcacggatgtgactgtaagtatttgtattccgtatccatcgtggatcagggaattactgagttttcacaatcatcaaaaagagagaagcattagttaaccttccctagttaggttcctttaattatcattttcatgtgtttctaaaaatctcatgctttaaacttcttgagattataaaactgagatgctttgtttaaacaagtgaattcttatttaaagaactagtcaagactagtgcttggtggtctttggtgtggggtcccagaggcactggctgctgtggccggcacatggcggggcagggtctgttcaccgcagggcagaggagcaccaaggcttcggtggctccccctcctaggctggcattcagccactgcacgctgatcggccactgcagctgcatctctgctgactggtcagggcccatgtcgcacccattgtaaatattttcaacatcacccctgcctcatcctcaatcacagtttgtagggtcctaggtgtgtatgaatacaggcaggatagagttgttaacttggtagcatcagaaaactctgtctgtattagtctgttttcatgctgctgataaagacatacctgagactgggcaatttacaaaagaaaggtttattggactcacagttccacgtggctggggaggtctcacaatcatggcggaaggtgagggacagcaagtcacatcttatgtagatggtggctggcaaagagagcttgtgcagagaaactcctgtttttagaaccatcagatctcccgacacccatctgcaatcacgagaacagcacgggaaagacctgcccccatgattcaatcacctccccccgggtccctcccacaacacgtgggaattatgagagctacgagacgaaatttgggtggggacgcagagccaaaccatatcaccatccttgcccatttttcagttttgctaaacattagattcagatgccagtcctttcttgccaaaataggctgtgaggcttctttctttcctatgctttattttctccaagacttaactgtatatgagggagaggggtatggtggcaggaggaaagagtggtttattttttggtccttggtcttctccaaatacagaagagactcctgttcttgaaaaggagggctttccatgtttgcatcttcatgactttaactgtcttttttaaaaattgacatacaataattatacatatttattgagaacatagtgatattttgatacatgtaatgtatggtgatcagatcagagtaattagcatacccatcatctcaaacatttatcatttcttcgtgttgggaactttctgagagagtgtaggctgtgggagataagtccgtcaccttttcctcctgatgtaaccagagtggctgcagccaggtcctcagaaactcagagagtacccagtgggaaatccctaagaccaaagtcagcatgggcttcagccatggcctgacaccatacaaaagaatgactgtccaacaagtgtatgaaaataagctccaattcactggtagtcaagaaatgcgaattaatgtaacaacaagatatttatctgcttttacccatcatactgcaaaactggaaaacagtgatagcacctgttgctggcaggccagtgaggaaaagtgtgctgtcctgagctgctggtggaaacgagagccatcaggcaatatctactgtaatttaaaatacttaataccctttgacacagatattttagtctttgggactctagcccatgaaaataaaagcagtaatgtgtgaagataggcacataaggatgtttgttttggtattgtttgtgtggtttaaaaaaaatccagaaagagagagggcaaatgccatcaaatggggcaatgtgtgaataaattatatttagccatggaatggaatgttctgcatgcagcttttaaaaaaatctgttagagctgtaccaagtgactcagaaggatttttgtgaagtataattaagtgagaaaaacaagataaaagtatgcataatacaatgccacttgtataaaacaaacaatggcaaaatctttgtatgactctgtttgcactcacccatgtttacagaggattgtatgagtgtgcagaaacaaatggaacaaccactcgggtgtccgtatggggaggatgggcaaagagactgatatgggtggagaacagagcagggctggatgagccaagcaaaaaaagttaaaacacagctggacctggtggctcatgcctgtagtcccagcactttgggaggccgaggagggagaatcacctgaggtcaggagtttgagaccagcctggccaacatggtgaaaactgtctctactaaaaatacaaaaattagctgggtgtgatggcacatgccagtagtcctagctactccggaggctgaggcaggagaatcacttgatcccaggaggtggaggttgcagtgagctgaggttgcgccattgcactccagcccgggcgaccgagcgagactccatttcaaaaaaagaaaaagaaaaaagaaaaaaagaaaaaaaaagaatcaccaaaacttatgtatatgtgcatacttttttgaaaatgtatgtctatgtgtagctatattctatatttacaaataaatgatgtcagaagaacaattggttaaaaaaatatgagaaaagaaacttcagtgccacccagcttacttccagcaagttgtaatggagaaggacatttccgtgaccatcctctctctgggacaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggtgaggatttagaaagctgatagcagtggcccttgaaactcatcttcatgtgttagagaccagtcctaccatatacaaagcagatcactgagtcagctccatgactagttacataggaagccctggattggcgtgaaatactggtgcccgaggttcctcctgccccttaggctcactgacagatcatcccaagcaggcttatcaggttgggtctaattttaaaacagtcattgaggagtcctggccaccccacccctgcttttgtttgatgcttcacctgtgtttgctgggttatggtgtacacagtaaatcctgtgtgtattttaaacaccaaaaataatgggatctgttgctggtctcttttacgaatttcaggtttcactgtgagacagaattcatttcacctcagtcccatgagcacttttgtgtgttctaatttctctacgacaccataatgggagaagacaccgatgcaacctgcggaggcctttctgcagacccacctttaactggttttctctctcccaacttgggctggccaggcactagcaagaccacactctgcataggaagaaaaagaaagtccctcccaaagctagattccttctgctttttctttcacgatccccaccccatccctcccaagtacccaaggatgttgcccgtgttgaatacatgtggttgcatcttcttcctccataggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatggtaagtcccatgtcagcactgtcgtgcacagcaaggagcatcctcttattaatacaattccagaacttttgagctagtgggcacctttgaggacagcctgccctggctgttttttatacagactagagataggaccctgagcaggcacgggaaggtctgcccaggcttcacggcctgggatcagttgagccaaggcttgagtcaggctcctccctcccagcccagagctctgtctttcctcctgtccttctgtcactggcaccaaactgcctctaatctcatcacttgagagtaatgactactcacctctgagaaggttccggggatggatgtagggcagcaaaaccaccttctgttcttttctgcacaaggactccttgtgccagctccaagcctctggcctttgaagaagtcccaagacctgtgttctccccctctccctcatcccatgaagtggagtgacttagagtgctccagcttcttgtccttccacccccagtaccaccctgaccaaacatggccccactgccaccggcctggagcaccctctcctctctgttaactggggccatggagcaccatattacctgagcctgcctgacccctgcaacatcttccctgatatgagccccagcctgtctcagtgaacatgaataacttgggcaatcactgtcatgctgggcgctgttcctggtcattgtccttagggttgaaaacagggagtctgatgaccatgagtgccacagtcagaagaggataatgcactggcttaggggtcttttctgagcatctgctgtttgctcaaccccactctgggcagcaccaaggaagggacagtggcagatgaaccatggaccttcccctcaggatgcttccagtctaatgcaggagccaggtcaataaagtatacgtggtatactcaataaggtgataagctgaacagtgcagacaagaagtcctgggcctgaccaggaaggagaaagaattattcatgtagctcagcgggcaacatttcatggaagatgtggagcaggaacccaaaaaatgcaaagaatatgtaaatgaaagagacatgtaagaatgggcttttgggcaaagaaaagttactgagcaggtgtgtgaggggctatgtggtgggatgggcatgtggaggatacaaagtttagacattgtccagtgagggtggaaaaagaggagtctacagcttgactcagctttggggatgccgacttgttgcaccccctggtctaaatgtcaagtacccagttatcttctttctctgagtttatctagtggtacaggactcctgctcccttctaccttgaaggtaaatgcttttaacagaagatacagggactgatcaaaatgctcgtctccaatctctttcatagacccgccccttgtcaactctgatcagcagcagccagtcctgtcagtacacactggacgctaagaggaagcatgtggcagaagccatctgcaaggagcaacacctcttcctgcctttctcctacaagtaggtcatgtgatgcacccctgatttgtcatttaatgggtcagtgtgaactgaacacttctcaagtgctctgttccaggcaaacctgtgcctgggagggaggaatggagagggataaaatgccgcccctccctgtccccctttttaagcgaacaggccatttggcagaaaagtcctaggcatgcaaaacaatccaagaccaacaaaagatatctaagacccattctttaagggctgtagatccagaaaacctgaggatcactgcagggtaccctggttagaaaaggtttcatggaagatttgggatactgactggaaacttgtgtatccaaatccactttgaaaactgataatcaatgaatatatattgagtaactgccatattcttggctctatgttgtggaagatacgaaagaattttgagacattgcactagttcctacctctggccactccagactagtggagagtataaggcacgcatgtctttttgatgggaggataactagcgtgaccaggaagaggtggatgttattcattcagggccaacaatggctggatttacccatgctttgaaagatgggcaggacttgggtagatgcagagacagggaaaaccttcaacatggaaagaatagtatgttctggccatccgtgacatggtgtgcttccttggttaccaggaataagtatgggatggtagcacaagtgacacagactttgaaacttgaagacacaccaaagatcaacagccgcttctttggtgaaggtaagagtttctgtccacatagttgctggaaaatctactcaagatgtgcctatcatggcttagccacttgctgagccctgttaaatgtctgctgactaacaagtgatacagacactggtgttctggctacctctagtgagaaagcaaactcatttcatgatgtcaagttgcaatggcataaaggaaaagaagttcccaaagctacttaggcatttgtaaatagaaaactggaatcctaagtttaacatgacatatttgatagaactgacatcacccatcctgtgataagatccagagctgtcccagacgaggtggaccaagtgggagagaaccttcagagtctggccagatagtaacctcaggagtcagtctttagaggtagaaggaactctaacaatctcaagtccaacccttacccagtattgtattgtatttatatctgtccaaattccttcttgtacattacctcattgtcctttttgctcatagcaacctgtgatgtcaggtggtagagatgtgattttatacctattctacagaggagacagtgacacagagaggcttagagtttgatgtagtcaaggccgcagaatattagaggggggaaaataagtgccaggttgtaatctaagccaggactattctcattacaccacatttccatgatgacttttacctctcttcctggcataggtcacagtaggtggtggagaggatacaaaagtgtctcccctccccacaagctgctggtagacccaattagaagaaatggtgataagcacccatgtgcctggtcccagttgtaaccatgtcaacagtagcacctcctcaccaattatttcaagctaagggtaacctgatgatagactcagacaagtctggattccactttagctctacctcttagaccctgagagctcttgggaaacctaagttgctcatctctgggtcacacttcctcatctctgggtctcatctctttgtctcatctctgggactcagagctgagatccagggatgagcaatttacatggcccaaaaactctgtgggtctcagaagcagggctgaatttatcattaaattgaacaataatgccaccccacagggataggatgatgagtcagtgaaaacaagtcaatcacctatggcagagccagatctagcaggcattgaatacaggatagtttctttcccttttcccctgtgctgatactccacaatttccagcttccagtagacaaagatatggttgagatgaagaaagctagagttcctttgacactttccatcttccaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccaggtatctaatggttacagctcaactttttataaaactgatggtaactgactgaactttcaaaccttggccaaatggagaatctcagggaccatttggatatcaatccagttaatcaattagtcaatcagttcatgattgctggatagagaactatcagctgctgcgctgagttccatgaaacacacacgcgcatactgtgttcaaggcagctatgtatttgtgtgttaaaacagaaggagaatagttcccacattttgatgggtaacttttaattcctaggtctattgcaggtgctctccagaagcttataggctggtggagagagaactcagacgaaaaatataatatgatttctctacccttcaaggcactggctttaagtgctatgaaggtgagagaagggactgaggccaggaatgagacccagctaatgttggccaggcatattctgtgtgctggccaaaggactgtgataacagtcttcttgttgctacagatccacagtcccctcttggaacttttctcgattgggcttcttctgtgggtaatattcctaaggaaagcatcatggttctgagctccaagttgggttttgaagttagatttgaatagtgaatgaggtgattaagggctctcctggcagaggacacaccatgagcaatattttatgtgccctgaaggtggtctgtataactttatccatgtctttcttctcagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaagtgagtttccacactgtatttctcctcctaggagcagaggaacatcttgcacctctgtgcatctctgtattaaaactgaacccctccttccactttcaaactctgctccttactcttgtgttttttcttgatcatttttggggtaatgacttgaaataagaaatcagcaaacacaaattgaatttttaaaaatattttctctacattatattataaaagtttttgaacatagcaaagttgacagaatttcacagggaaaacccctagaaaaccagctatctcctactatttaagtgttattatatttgctttatcacatatacatccatccattaattcatcttattttctgaagcatttcaaagtaaattgcaaacatcaacacactttcccctaagtattacagcttgcatattattaacttcagttcaatattagttagcagttttttcctctgaatttttttgtttgtttgttttgtttttttttgttgttgttgtttttttgagatggtctcactgtgtcacccaggctggagtgcagtgatgcagtcacggctcactgaagcctcaaattcctgggctgaagtgatcctcccacctcagcctcctgagtagctgggaccacaggtgcatgctaccatgccctggctaatttttgtattcttggtagatacagggtttcaccatgttgctcaggctagcaggtttttcctttgatgaaattttttggctttttcttttttacatttttatataaatttatgtggaacaagtgtaattttgttacatgaatagattgtgcagtagttaagtcagggctttcagggtatccatcacccagacaacatatagtgtacccactaagtaatttctcaccatccatctccctccacttccacaccttctgagtctcaattgtctatcattccacacactatgtccttgtgtgcacattatttcactcccacttataaatgacaacacgcaatatttgtctttctgtgactgtcctgtttcacttaagacaatgacctccagttccatccatgttgctgcaaatgacatgattttattctttttatggccgaatagtattttattgcctatacatttcacatttttaatccaatcgtccattgatagacacttaggttgattccatgtctttgctattgtgaatagtgctgtgataaacatatgggtgcaggtttcctttggatataatgatttcttttcctttaggtatatacccagtaatgggattgttggatttattggtagttctatttttagttctttgagaaatctctgtattgttttccatagtggttgtacttatttacaatcccatcaacagtgattaactgtttccttttctctgtatcctcaccaacaactgttattttttgtcttttgaataatggccctcctgactcttgtaagatgttatctcattgtggttttaatttacatttctctaatgattagtaatgttatgcattttttcatatgcctattgccatttgtatgtcttcttttgaaaaaaatgtctattcatgtcctttgcctactttttaatgggattatttgggggatttttttgttgagttgtttgaattgcttgtacattccggatattagtaccccattggatgaatagtttgcaaatattttctcccattctgcaggttaccaccctgttgattatttgttttactgtgcagaaactttttactttaattaagttctatttgtctattttttgtttttgttgtctttgcctttgaggtcttattcacgaattctttgtctaggccaatgtccagagaagttttccctaggttttcttcttgcatttttatagtctcaggtcttatatttaagtctttgatccatcttgagttgatttttttatatggtgacagataggagtccagttttattcttctgcatatggcaatccatctttcccagcaccacttattgaaaagggtgtcctttccctagtgtatgtttttgtcaattttgtcaaagatccgttgactgtaagtatgtgactttatttctgggttcagtattctgttccattgatctatgtgtctatttttatgccagtaccatgctgtttagattactatagccttgttgtataatctgaagtcaggtaatgtgatgcctccagctatgttctttttgcttaaaattgcttcagctattcaggctctttttggattccatatgaattttataattattttttctaattcacaagtttgggttttaagacaaacctaactggggttaccaagtcctgactctcttctcttattctgtagctatcataagacaaaccctacagggacccaggagctgctggacattgctaattacctgatggaacagattcaagatgactgcactggggatgaagattacacctatttgattctgcgggtaatctcagtcttttatatgacatacatcatttcagaagcacttttcctggacaccttttacttccctctcctgcaccctgatgggttcttgtttcttttcttcaatgcaggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggtaaagtccacaagaagaggtctgaaagtgaaagtttattaacaaggatttggaaggtactaggggaatgagactctagatttcatctactgactttattctgctgtttctttcctttccttccttccttccttccttccttcctccctccctccctttcttctttccttccttccttccttctttcgagatggaatctcactctattgcccaggctggagtgcagtggcatgatctcggctcactgcaacttctgcctcctgggttcaagcaattctctctgcctcagcctcctgagtaactgggattacaggcatgtgccattacacccagctaatttttgtattttttagtagagatggagttttgccatgttggccaggctggtcttgagctcctgacctcaggtgatccgcctgcctcagccttgcaaagtgctgggattacaggcgtgagccactgcacctggcctctactgttttctaattgcaaatttcaacaagcctattgacttgactgcctagcagtatgtgacgtgagagaaatacttgactttgctgctatgtcaacatgcagaacgtgagatgtttttgcttcctaccgtccacctaccagattgaccatccctctcatcatggaaaaacatgcttaattttcccccaataagcttaggctaggatagccaacttggccccctcttaggtgcaaagactccagaactttggaaactaccctatttattagccccaaactcttactaccccttctcatctttatcctcacattaaaataacttacgttaaaacaacttgattttcacttagtggtggatctccaaacaaatcacaacttggccataatttatgtgttttaatggaattgaattcaacaggcattccacaggctttttctgggaacccttacttgatagtgctctaggaaacactggcaagaagattcaataccagcatttgaagaacgattacagagaaattagacctgtgcttaagaaagagctagcagacaatgccagtgtttgccaggcatgttctgtgttctgaccacaggacagtgataaccatctcctcttttgactgcaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaagagtaagtaagagctattcaccccatataccactgagggccctgagctggaattccaaccctaggttttggcatagccactgtctgcccttgcttctgaaacaaacacttgtgcaaatgtgtagcagatctagacccaaagacttagggtcaatgaaatcaagacattttggtagtgattggaaatccatatttacttggggtgcaagagtcaaaggataataacatggtgtgtcagctcaaaatatacttcttcttatctagtctgaaaaagttagtgaaagaagctctgaaagaatctcaacttccaactgtcatggacttcagaaaattctctcggaactatcaactctacaaatctgtttctcttccatcacttgacccagcctcagccaaaatagaagggaatcttatatttgatccaaataactaccttcctaaagaaagcatgctgaaaactaccctcactgcctttggatttgcttcagctgacctcatcgaggtaagtgtgaagagtttgaggttctctagcccattttgtacagcatcataaacagagagtccctgggagccaggagctacccagaggaaaactaagaaccaccaggcacttcctaccatgattctgaggctttcttctttccctccttccccgccttcctctctccccgctaggggtcacctgaagcatgacttcttaacattaatagaaatgcaggcctggcgaggtggctcactcctgtaatcccagcactttgggaggccgaggcgggtggatcatgaggtcaggatatcgacaccatcctggctaacacggtgaaagcccatctctactaaaaatacaaaaaattagccgggcgtggtggcaggcacctgtagtcccagctacttgggaggatgaggcaggagaatggcgtgaacccaggaggctgagcttgcagtgagccgagagattgcgccactgcgctccagcctgggcgacagagcaagactccatctcaaaaaaaaaaaaaaaaaaaaaaattgaaatgcaaatgtctcgtctttaagtcccaaagccaaggaagcatatgtgctgcctagtcagatctgcttcaaatctcaaatcactcccaactctgaatcctttgttgaattatttgtcctatctgaaccttagctgcctcttctagaaaaaagcaagtaataaggtcaagattctagtgagattttaataaagcagctcctgtgaaatgctaaggtcagctcctggcctgtggtattcaaatacttgtttagataaatggacatcaagagtggggactactaggctggcatacaacaaagaaacctgatgccattttcttgtctgattttctttctcagattggcttggaaggaaaaggctttgagccaacattggaagctctttttgggaagcaaggatttttcccagacagtgtcaacaaagctttgtactgggttaatggtcaagttcctgatggtgtctctaaggtcttagtggaccactttggctataccaaagatgataaacatgagcaggtgtgtatttgtgaagtatcttcttaaggaaagctttgggtctcaatgcaaaaacaattcttttctaagcatggaagtcctcaaaatactatctaactgaagggataactatggtttttatcaaccagacctgctggggtaagggccagtatcctctgcagttaaagatctcctgaattcagtgtgcccagaaaccagactcacaataagtactctaggataacaagagtatgaactctgggctgggtgtggtggttcatgcctgtaatcccagcactttgggaggccaaggtgggcagatcacaaggtcaggaatttgagaccagcctggccaacatactgaaaccccgtctctactaaaaatacaaaaaaactagctgggcatggtagtgggtgcctgtaatcctagctactcgggaggctgagacaggagaattgcttgaacccgggaggtggaggttgcagtgagccgagatcacgccgttacactccagcccgggtgacagtgtgagactgtatcttaaaaaaaaaaaaagtatgaactctgggcatagatttaattctaacttccctgtcttgaagctgtgcgcacttggggaagttggttgatattatgtgtatctgtttctgtctgtatcccagactactaataacagtccaaacctcacaaggttatttaaagacaatgaaataaggcatctaaaatgccaagcacagtgcctgatgctggcattggttgttcaataagcagacactattacgagttctaaattaatattttcattattattaactgctgtctttggctctcactcccatcagtgcactagcaaatgagaccaaacttccactttgaagctagcaatgagcccccatttaaggagggaaataggttgtatgatctggagcttattcttgaattttttgctacccaaagtgtggtctggtcagaaatacagcttctcatgcttcacccacaatctactgaatcagaagcgcattttagcaagacctcatgtgacttgtatgcacattcaactttgcagagcaaggcagtaatttacccctccaggctcactgttgagcacgagctccatcttctaatttcctgacccccacttgaggccgaggatctttgatctgctttgagtctgtcagtttcacattttttttttcccaatgcctgggcatccatctctgagattcttcttctctctgagaagaacttgtctaggatcaagtgtttttcaaacttctggtgaatttatataacagctacattttcttaagaaacaccttgtagtcttcactggtcaaagaagagaaggctaagcagggaacgggtgggggatagaggatcttctaatcttgaggatcctggcatactggagaatagggacccctcctctcatcccaccacatcttactatgtctacagattttttaattaagaatagctttaggagtgccactatccctgacaagaccttagttctttaatctctgcttagaggaattagcctggacttcagtgtctccctgttcctcacctggagcattttttaggcccatcctggctgcatcagacaggtcccacattgggaactgaaaggtgtttgacattgctgacatctcactggccattttattactaaactctcaggatatggtaaatggaataatgctcagtgttgagaagctgattaaagatttgaaatccaaagaagtcccggaagccagagcctacctccgcatcttgggagaggagcttggttttgccagtctccatgacctccagctcctgggaaagctgcttctgatgggtgcccgcactctgcaggggatcccccagatggtaagtcagcaggccccactgggggcccatgagaccagacgttggtttttttttagatcgcccagactcccttacgatcccagctgcacaagcccgaaaagatgcttgtactttcttcagagatggaggtttgccttgaatttcactgaagatgactcttggatcacatggaaatgttaacatttagaaattaagctattcataatgttagctgtatttttaagagcattaatttattcatctggaaaacaatgttcggtataccttcctctacctttgctgaaggtccttttatttttatttttatttttttaattttttgagatggagtcttgctcccaggctggagtgcagtgatacaatctcggctcactgcaactctgccttccgggttcaagcaattctcctgcctcagcctcccaagtagctgggactgtggacgtgcaccagcatgcccggctaatttgtgtatctttagtagagacaagcctgttgacaaccatgtcaggctggtttcgaactcctgacctcaagtgatcctccagcctgggcctcccacagtgctggaataacaggtgtgagccactgcacctgacctgaaggtccttttaagattgaaatgatacaatgattataaaagaaagtatttggcaaactataattcactatctaaatatgctataatttttattattaattcataaaaggaaatatataaatgtactcctatggcttgattaaaaaaatgttgactttaagaaaacaggtctcaagctattttattgaaatattatttaaaaaataaaacccaatgcaaattgatatgtacatcatctcaataggcctttggtttcaaaaaattgattttatcataatataatacatttcaagtacaccttcacttacagtcagactccagaacaccagaattaagccatggcatatatgatacttaaagtccataaagctctgaggcccagcaatattcttaagagccttctgagtccacttgaaaatgacatgatatctatctagtgaaatttcttatatcctgattcactgaaaacggtaaaaacatcagtttgatctttatttatcaaactattcagctcatcaaaatatgctagtccttcctttccagataaagaggaattactctccaatgtatgggaggttgtaattaacaaaaccgactttaaaaagacttacttttatttgctctcccttgttgggtctacagattggagaggtcatcaggaagggctcaaagaatgacttttttcttcactacatcttcatggagaatgcctttgaactccccactggagctggattacagttgcaaatatcttcatctggagtcattgctcccggagccaaggctggagtaaaactggaagtagccaacgtaagattctgtttgccttttgatttcttaggttattactttcttccagggtgcatttcttgttaaaacatatttaaaaatgtgtttccacttcaagacaaaatgcttcatcattgtaatcacctcattatttttttatgaaaaacttcaagcttccaccagaatgcactacctcactagctccagtagtggtatggccataagacaagaactcagttctctcaacaaatgagtattcctatcatctttttaatctggttttgcctcacgttaactcaggtgctttctagttctgggtagtatactccaactctagagaactgagaactcgctttccttcttccaaacaaatcccagtaatgtttccaaaggtctgagttatccaggaaatctttgcccggaggtgagaaagggtggttgatctgactgacaggggactgaagtatttaatgaatctgaataggttgttttctgacttatagatgcaggctgaactggtggcaaaaccctccgtgtctgtggagtttgtgacaaatatgggcatcatcattccggacttcgctaggagtggggtccagatgaacaccaacttcttccacgagtcgggtctggaggctcatgttgccctaaaagctgggaagctgaagtttatcattccttccccaaagagaccagtcaagctgctcagtggagggtaattctttcagccaagtctgcctagccagtttgaaagagagaacagagaatgtacctgcagaattttgccaggctaaacagttgattgagatcattcaggtcctgaggaagcaggagaggagtagaaaggaaagattccgggttacctattttaattctagcctagacttactacataactacataattacctttcttctacttttcacattttactaaactgtcctttatctttctgctttgagacttattaagacctactgcttaattagtttttattaagttgtgattttttgttatctatttgttttgagaatgaagaaacaatagctctggagagatcatctttggaaaattaatattttcccccccaaaaaatacctaagaacatattgatttgaggtagctaggtaggtaaagcatgaaactcctaacctcgtgataatggaatacagcctcttttggagagttccattttaagtggcaccctcaaccattgatttgccttagttttcatattttagacacattcatgtgttcattcaaaaataatatttaattggccagccacggtggttcatgcctgtaatcctagcactttgggagccccaggtggatggatcgcttgagccctggtgtttggataccagcctgggcaacatggcaaaaccccatctctacaaaaaaaattaaataaataacaaaattagccagtcgtggtggcacatgcctgtagctccagctactcagaaggctgagatgggaggatcaactgagcccaagagttcaagccttcagtgaaccatgcttgcaccactgcactccagcctgggagacagagcaagatcctgtctcacaaaaaacaaaaaatagtatatttaattgcctaatatataccacgtatgttgagtgagacacacaaggtccctgacctttgaacgcttacattttataagggagacacacaattaagcaagcagtaatcatagagtaagggctaagttatagaaagtattagagtaccatgaaattttatatcatgtagcctgtgctagtcagggaatgcattctgaagcaagtgtacttgacctgataactgaggactgtgtcagagtcatttaggcaaaggagaaaggagtgagtgttccaggcaaaaggaaaagcatgtaatggcctgaaggtaaaggaatatggttcaaggaactggaagaagtgcagaatggtaaggggctcagagatgatggggagaggtaggcaggggagagagcatgcccagctgcgaaagccatcctaaggagtttggactcttttgaaggcacaggagttgaaaaggggagcagaaataagataggggtgatgttttagaagaaatactctgactctagtgtggaagatgggtgagaaggaggcacagctggacacgaagagaccattggacatctcttacgatcctatgtggctaagagctgataatggcctgcagtggagaaaagccaggtatagaaaggagtgagcagattctacaactttctaagaggcagaatcataagtactgggtgattaactgggtatggggacaaggcaaaagaaagaagaaaagaggaaggaggcgcccttcattttaataagaactacagtgggagagcttctggtttcaaggaaagtgacaaattcagttttggatgtgctgtatttgatgtcctcctatgaaacaaccagtttagaaatctagctgtcaaatagacctatggatctgagcccagtaaagaggcttgggctccacatatggatttgggaatcattagtatacagaggttgttgtggttaaacagcaactggtatagagtgagacatgagagatgaggacagaaatatggagaagacaaacatataaaggaagaaggggaataaccagcaatgagttagaagaagtgaccagagaagcagaaggagaaccaaagccataaaaggtcacagaagccaaagagcagccacaggggagatcaccccatgggtaggcgaaagctggcattaggactccagcacatcagcaaagcttggtcttgtggcacccccaacttggagaaacaatacttggaggaaaatgtgctatttcaaagaaagcatccttagaaaaaaccaggccaatgttgaactttcttacatgtactaagtttttaagtacacacttggaaggaaggtgccatcatctcttcagatgtgagaggctccagcgtcttagtctggtcatgagtgcgcaactctatggaaggcttctgggaggtcaaggaagatgaaacctaaatatgcccattggatgtaggagcaaggagggcattagagacattgatgaaagcattttcaggagatggagtgagcagtcagagcacattgggaggaagtagagactgcaaaggcagacaactcttgatggtgaggaagatgagaaagcaagaaaagaaagaaaggagcataggggaggggcacaggggaagagacttgagcgtgcttaatgcaggtggaaggaagcaggtagagagtaggagatttcatatgaaagagacagtttctcttgccctgcattgtaggaaggaaggggcacactgaagttcagccccagtgatcagctatttaacatctctgagcctctgcttctgtaaaatgagaaccataagcctactgttgtggggattacaggtaacagatggaaagaactcagccagaagcttcagagtcactctcatggcttgtcatgttgatgttctttctaatattatttgtttctcagtaaattaaatagttagagataggtgtggactgagggaagacaggaggataagggggtatttgcaccctgagaatttgtgatgtccattttgattcatgacttggcaataactcaggtatttttgttcttcaccagcaacacattacatttggtctctaccaccaaaacggaggtgatcccacctctcattgagaacaggcagtcctggtcagtttgcaagcaagtctttcctggcctgaattactgcacctcaggcgcttactccaacgccagctccacagactccgcctcctactatccgctgaccggggacaccaggttagagatgctcagtgcctgacccagcattttctcaccttccacatcatggccacctagcatggcacaggaaaaaatactctgtgttgtaagaccctgtcactagccttctgggtttgcaccatctttgggtatttaaagcagggtcctctggccaacacattgggtgtcaccttttgcttccttgtgcatgggatgggatcacagcacagatcccaatttgctcctaattcagtgtccatgtttctgagcctccagacccatcgctatgagcttcctggagcccaccaatgtgcttgaagccttcaccgtacttaggtggctccctgtcttcagcccccaagttccagtgcttgttctcagctttgctgaaacaaccagccaactcctgctctgcttgtccaaagtcttgggaatcctggtgtctgcccttgccttgggttcttgtaggactgagggatcaaaaagatcatcttagttaagggcaagagacaatgttaaaataaggaccatatttttgttgcatttgaggctgaattgttttgggaacataatcaccatccttgaaagctctaacattatgcactgtcttcattgtaatgtctttagattagagctggaactgaggcctacaggagagattgagcagtattctgtcagcgcaacctatgagctccagagagaggacagagccttggtggataccctgaagtttgtaactcaagcagaaggtgagtattcaaaacacagctgcctcatctctgctcgcagtctcaggttcagaattcatgaggagaagacatgtaatttaacctatttaacaaataggttaactgagtacccactaagcggcaggcctattctaagacctgggttaactgagtacccaataagcggcaggcctattctaagacctggggctagaacagtgaacaatggagtctctgccttcatggaagttacagtgaacaaccaaacaagttaatatttggaatatcagataagtactgaggaggaaaacagagcgtagactggtctatggagggctaggagtaggagggaggaagaagggcagggaaagcagtgcatttggaataataagggaaagtctccctggtaaagtgagcataaggagacctatcagaaataagaggagaagccgtgtggtaagactgttaacaggcagagggaccagcaagtgcaaaggccctgaggctgacacactactaccatgtttcaaggaaaggaaggaagacagtatggctggagcagaaagaccagggagaaaagaggtagaagatgaggacagagagatatggagaggtgaaggaaggataatctcataggccatggtaagaactttggctttttctatgaattaaacgaaagccattggggagtcctcatgatttgatttatgtttatgttgagaaaagactatgggcagacaagggcagagaaactaatatgtaggttatcacaataatccaggcaggaatcagtgttgttttggatcagggcaatggcagaagagatatgagaaggggatggattctggccatattttgaagattaggctgacaagatttgctgatacagtggatgttgagtgtaagaggaaaaggggaatgaagacaaacctaaggtttttggcccgggcaactgaaaaatggaacttccatttattgagatggaaagggctactggaggagcaggttttagggaatgggagaaatttaggtgttcactttggaaaaaaaattatatagggatagcgaggagcaggttttagggaatggggcacatttaggtgttcactttggaaaaatttttatatagggatagcatatcacagaattaaactaggaagaaaatcccatgatagaaagcactggaggagcagggcacgctggggaaatagtgtttggtaaacattgttttacgaaggatataaaatggaccagcctatggattgaaggacgcccgggaatcttgttacaaagaaagggggagttggggagatggagcccagggcaagggcagcaaggaaccaggacaggcatcttgggtagaaagtaatatagagatgtcgtgtcttcctggcccagaagggctgcgagcctttgctgttccacaaacaagctaagtgctccccatttcagggcctttgcattcctgaccttctgcctggaatgtgctcctcccagaactcagcgtggctccaacctcttttcattctggtctctgcccacatgtgcccttatcagagagaatttctctgaccaccaagtatgaaataacacttcttctatccctttcttttatccttgtatccagttttactcttcttcataacattcattaccatctgacatgagcaagttacttgtttattgcctgtacacctcccccactagaaggtaagccccatgaaagcaaggattccccagtaccaagagcagtgcccagcacacaataggctcataacaggcaatccataaagacttgcatacatgaacacaactgagtttaaaattatcagtaaatgagacccattaaaaaattttaatgagaaaaaaaaaattcagtaaaatcctgaactgtgtttttgtttaagcacattgattccttggagtttctctaccttttcctctctttccttccaaaacatagcttctttatttatttatttatttatttgtttgtttatttatttatttatttatttatttattttttgagatggagtctcgctcttttgcccaggctgcagtgcagtggtgccatctcggctcactgcaagccccgcctcccgggttcatgccattctcctgcctcagcctcctgagtagctgggactacaggcacccaccaacgcgcccggctaattttttgtatttttagtagagacggggtttcaccatgttagccagaatggtcttgatctcctgacctcatgatctgcccgccttggcctcccaaagtgctgggattacaggtgtgagccaccgcacccggcccaaaacatagcttcttaccacacatctcttgattctcttatacactcgtccaggtgcgaagcagactgaggctaccatgacattcaaatataatcggcagagtatgaccttgtccagtgaagtccaaattccggattttgatgttgacctcggaacaatcctcagagttaatgatgaatctactgagggcaaaacgtcttacagactcaccctggacattcagaacaagaaaattactgaggtcgccctcatgggccacctaaggtaaagaaggccgagggtcatctgacctgcactgcaggcctgggtggttcttttcattattcctcttccacttcatacctgaccaagccatgttctcccctagtctacaatcagagtggcagagagagccctcaacaattttttttttttttgagatggagtctcactctgtcaccaggctggagtgcagtggcacaatctcggctcactgcaacctccgcctcccgagttcaagtgattctcctgcttaagcctcccaaggagctggaactataggtgcatgccaccacacccagctaatttttatatttttagtagagacagggtttcaccatattgaccaggatggtctcgatctcctgacctcgtgatccacctgccttggcctcccaaagtgctgggattacaggtgtaagccactgcacccggccaagctctcaacattttaaccctctgcgcatgtccagttggattttcctaccatttatcaggcacttactattcatgtatcaagcacagtgctgggtgctttaaagaaattatctcggtcctcacaataaactgcgaggtcactgtgagttttcctgtttcatggataaggaaatggtagctcagaggggttaaatcatttggtcaaaatcacagagctagtaaatagcagagcaggattcaaacagttttcaaaaaacttctctttctcctaaacctgtttgcaaagtccttaatttgtgctgaatgttggctttagaagttgatgagtttgatctgtggctgtttctctgaaccatccttgtatctggttttgatcaccacaaatggaacttctgtttaatcctgcatatctccattgaaaggacaaaatcattggtgccaactgattttctttaccatagttgtgacacaaaggaagaaagaaaaatcaagggtgttatttccataccccgtttgcaagcagaagccagaagtgagatcctcgcccactggtcgcctgccaaactgcttctccaaatggactcatctgctacagcttatggctccacagtttccaagagggtggcatggcattatggtatgtgtctcttcccctgtgtgagcacttccaaagtaatgcaggtgttgagacctgtggttacaggctgaactagtaccattcacaactatttcctacgtattttcagatgaagagaagattgaatttgaatggaacacaggcaccaatgtagataccaaaaaaatgacttccaatttccctgtggatctctccgattatcctaagagcttgcatatgtatgctaatagactcctggatcacagagtccctcaaacagacatgactttccggcacgtgggttccaaattaatagttgtaagtatgagtctgccagtcaataaatacatggatataagtgctaattacatcctcaactctgagctaggtgcaggaaggtttccaaagatgtataaggcatgcttccttccccccagggaattcttggggagaaaaaaaaactttcacaagtgtgtagttacccagttacacaaagctgaatgtgatacatatcaaagagatgctactaagtagaacagttctttgcctagtggtatcaaaggaagcttcaggacaccagctaggaggctgactatgttagacattccttttataaatatggacagtgatcagtgactggcaacgaagattcataattttctgttatttatttttaactttcagtgcattgtccagcttaataattaacttgtcaaatcggtatttttgcctaatgttcattgctctttgaggctcatccaagcccattaccttaaaaatctcctgtcattttgtaggcaatgagctcatggcttcagaaggcatctgggagtcttccttatacccagactttgcaagaccacctcaatagcctgaaggagttcaacctccagaacatgggattgccagacttccacatcccagaaaacctcttcttaaaaaggtaaaagaagaaagcagcaaggcttcttgaaccatgcaaagtaaatgaaagattttacatagcatgatttagacatttttttaaatttttaaaggaaataatttaagcattttaaggagattaataactatagcacaaacactgtggcatctttgcattagtaaacatgagaacaccaaccctgtcaggaagaatctaagaaagtcattagaggattctggtactttcaccctaagatattttattcagtacaacctgttataagcaaattctccctctgactgtgaagaattcagaatggctagaggcgttattgactacaggcttgctgttaagctagagagagtcagaacagccattgagcactaaatggaggcagcattctgagaaaatactttaacccaggcttactgacttccatacctatgttctttccacaaatcaagttgtctcaattcagtttagcaaatttgtatcaagtatcccctatgtgcaaaatgctagactaggtacagtgagaagatagaaactgggtaaggtatagccttttctttcaagaagataccatggagacatcaacaaatgagaaataattaattatataagcaaaattatgacatgctctttgagaaaggtgcaagggactatgtaactgtaagaatgagacaaattggctatgacttaggtgggatggtaatgataaggagtggcccttagaagagctttgtcaggatttgagtgtttgacaggtggaggtaaaagcaaaggggtccaggcataggagtagcacaaagaaaagtgcagagtggctttgggaatggggcaagtacaatattgttgtgaaggtcagaggcagagaactttgaatgactgatgtctgactgtggggatgttatctttgttgttcatttcagcgatggccgggtcaaatataccttgaacaagaacagtttgaaaattgagattcctttgccttttggtggcaaatcctccagagatctaaagatgttagagactgttaggacaccagccctccacttcaagtctgtgggattccatctgccatctcgagagttccaagtccctacttttaccattcccaagttgtatcaactgcaagtgcctctcctgggtgttctagacctctccacgaatgtctacagcaacttgtacaactggtccgcctcctacagtggtggcaacaccagcacagaccatttcagccttcgggctcgttaccacatgaaggctgactctgtggttgacctgctttcctacaatgtgcaaggtgagctatgctcaggtaaagggtgcaccgggctagttcatggcaggctctaagaggagagcctcctccagggaggaaaggactttggctttctagcagataatcttccttgctacttggaagtcttttattttattcaacaaatagaaatatttattaaacatatcacgtgtattaaatattctagtaggcagtaacagaaagtagacagataagccagcaattataattcagtgtgagaggtgctatgataaagtgtagtatataagtataaggtagagtggaagcactcaacaagggaacctaaacaaagcctgtggtggtcaggcaaggcttcctggaggaatgccttttgctatcagattttatctttgcattacagatggaggagtctattgcacaattggcccagaaaaatggggctttattattgaaagactttcaacatagagattgctctggaaatgtactgcttaatttaaccaatgtcttttcatttttatgttaggatctggagaaacaacatatgaccacaagaatacgttcacactatcatgtgatgggtctctacgccacaaatttctagattcgaatatcaaattcagtcatgtagaaaaacttggaaacaacccagtctcaaaaggtttactaatattcgatgcatctagttcctggggaccacagatgtctgcttcagttcatttggactccaaaaagaaacagcatttgtttgtcaaagaagtcaagattgatgggcagttcagagtctcttcgttctatgctaaaggcacatatggcctgtcttgtcagagggatcctaacactggccggctcaatggagagtccaacctgaggtttaactcctcctacctccaaggcaccaaccagataacaggaagatatgaagatggaaccctctccctcacctccacctctgatctgcaaagtggcatcattaaaaatactgcttccctaaagtatgagaactacgagctgactttaaaatctgacaccaatgggaagtataagaactttgccacttctaacaagatggatatgaccttctctaagcaaaatgcactgctgcgttctgaatatcaggctgattacgagtcattgaggttcttcagcctgctttctggatcactaaattcccatggtcttgagttaaatgctgacatcttaggcactgacaaaattaatagtggtgctcacaaggcgacactaaggattggccaagatggaatatctaccagtgcaacgaccaacttgaagtgtagtctcctggtgctggagaatgagctgaatgcagagcttggcctctctggggcatctatgaaattaacaacaaatggccgcttcagggaacacaatgcaaaattcagtctggatgggaaagccgccctcacagagctatcactgggaagtgcttatcaggccatgattctgggtgtcgacagcaaaaacattttcaacttcaaggtcagtcaagaaggacttaagctctcaaatgacatgatgggctcatatgctgaaatgaaatttgaccacacaaacagtctgaacattgcaggcttatcactggacttctcttcaaaacttgacaacatttacagctctgacaagttttataagcaaactgttaatttacagctacagccctattctctggtaactactttaaacagtgacctgaaatacaatgctctggatctcaccaacaatgggaaactacggctagaacccctgaagctgcatgtggctggtaacctaaaaggagcctaccaaaataatgaaataaaacacatctatgccatctcttctgctgccttatcagcaagctataaagcagacactgttgctaaggttcagggtgtggagtttagccatcggctcaacacagacatcgctgggctggcttcagccattgacatgagcacaaactataattcagactcactgcatttcagcaatgtcttccgttctgtaatggccccgtttaccatgaccatcgatgcacatacaaatggcaatgggaaactcgctctctggggagaacatactgggcagctgtatagcaaattcctgttgaaagcagaacctctggcatttactttctctcatgattacaaaggctccacaagtcatcatctcgtgtctaggaaaagcatcagtgcagctcttgaacacaaagtcagtgccctgcttactccagctgagcagacaggcacctggaaactcaagacccaatttaacaacaatgaatacagccaggacttggatgcttacaacactaaagataaaattggcgtggagcttactggacgaactctggctgacctaactctactagactccccaattaaagtgccacttttactcagtgagcccatcaatatcattgatgctttagagatgagagatgccgttgagaagccccaagaatttacaattgttgcttttgtaaagtatgataaaaaccaagatgttcactccattaacctcccattttttgagaccttgcaagaatattttgagaggaatcgacaaaccattatagttgtactggaaaacgtacagagaaacctgaagcacatcaatattgatcaatttgtaagaaaatacagagcagccctgggaaaactcccacagcaagctaatgattatctgaattcattcaattgggagagacaagtttcacatgccaaggagaaactgactgctctcacaaaaaagtatagaattacagaaaatgatatacaaattgcattagatgatgccaaaatcaactttaatgaaaaactatctcaactgcagacatatatgatacaatttgatcagtatattaaagatagttatgatttacatgatttgaaaatagctattgctaatattattgatgaaatcattgaaaaattaaaaagtcttgatgagcactatcatatccgtgtaaatttagtaaaaacaatccatgatctacatttgtttattgaaaatattgattttaacaaaagtggaagtagtactgcatcctggattcaaaatgtggatactaagtaccaaatcagaatccagatacaagaaaaactgcagcagcttaagagacacatacagaatatagacatccagcacctagctggaaagttaaaacaacacattgaggctattgatgttagagtgcttttagatcaattgggaactacaatttcatttgaaagaataaatgacgttcttgagcatgtcaaacactttgttataaatcttattggggattttgaagtagctgagaaaatcaatgccttcagagccaaagtccatgagttaatcgagaggtatgaagtagaccaacaaatccaggttttaatggataaattagtagagttggcccaccaatacaagttgaaggagactattcagaagctaagcaatgtcctacaacaagttaagataaaagattactttgagaaattggttggatttattgatgatgctgtcaagaagcttaatgaattatcttttaaaacattcattgaagatgttaacaaattccttgacatgttgataaagaaattaaagtcatttgattaccaccagtttgtagatgaaaccaatgacaaaatccgtgaggtgactcagagactcaatggtgaaattcaggctctggaactaccacaaaaagctgaagcattaaaactgtttttagaggaaaccaaggccacagttgcagtgtatctggaaagcctacaggacaccaaaataaccttaatcatcaattggttacaggaggctttaagttcagcatctttggctcacatgaaggccaaattccgagagaccctagaagatacacgagaccgaatgtatcaaatggacattcagcaggaacttcaacgatacctgtctctggtaggccaggtttatagcacacttgtcacctacatttctgattggtggactcttgctgctaagaaccttactgactttgcagagcaatattctatccaagattgggctaaacgtatgaaagcattggtagagcaagggttcactgttcctgaaatcaagaccatccttgggaccatgcctgcctttgaagtcagtcttcaggctcttcagaaagctaccttccagacacctgattttatagtccccctaacagatttgaggattccatcagttcagataaacttcaaagacttaaaaaatataaaaatcccatccaggttttccacaccagaatttaccatccttaacaccttccacattccttcctttacaattgactttgtagaaatgaaagtaaagatcatcagaaccattgaccagatgctgaacagtgagctgcagtggcccgttccagatatatatctcagggatctgaaggtggaggacattcctctagcgagaatcaccctgccagacttccgtttaccagaaatcgcaattccagaattcataatcccaactctcaaccttaatgattttcaagttcctgaccttcacataccagaattccagcttccccacatctcacacacaattgaagtacctacttttggcaagctatacagtattctgaaaatccaatctcctcttttcacattagatgcaaatgctgacatagggaatggaaccacctcagcaaacgaagcaggtatcgcagcttccatcactgccaaaggagagtccaaattagaagttctcaattttgattttcaagcaaatgcacaactctcaaaccctaagattaatccgctggctctgaaggagtcagtgaagttctccagcaagtacctgagaacggagcatgggagtgaaatgctgttttttggaaatgctattgagggaaaatcaaacacagtggcaagtttacacacagaaaaaaatacactggagcttagtaatggagtgattgtcaagataaacaatcagcttaccctggatagcaacactaaatacttccacaaattgaacatccccaaactggacttctctagtcaggctgacctgcgcaacgagatcaagacactgttgaaagctggccacatagcatggacttcttctggaaaagggtcatggaaatgggcctgccccagattctcagatgagggaacacatgaatcacaaattagtttcaccatagaaggacccctcacttcctttggactgtccaataagatcaatagcaaacacctaagagtaaaccaaaacttggtttatgaatctggctccctcaacttttctaaacttgaaattcaatcacaagtcgattcccagcatgtgggccacagtgttctaactgctaaaggcatggcactgtttggagaagggaaggcagagtttactgggaggcatgatgctcatttaaatggaaaggttattggaactttgaaaaattctcttttcttttcagcccagccatttgagatcacggcatccacaaacaatgaagggaatttgaaagttcgttttccattaaggttaacagggaagatagacttcctgaataactatgcactgtttctgagtcccagtgcccagcaagcaagttggcaagtaagtgctaggttcaatcagtataagtacaaccaaaatttctctgctggaaacaacgagaacattatggaggcccatgtaggaataaatggagaagcaaatctggatttcttaaacattcctttaacaattcctgaaatgcgtctaccttacacaataatcacaactcctccactgaaagatttctctctatgggaaaaaacaggcttgaaggaattcttgaaaacgacaaagcaatcatttgatttaagtgtaaaagctcagtataagaaaaacaaacacaggcattccatcacaaatcctttggctgtgctttgtgagtttatcagtcagagcatcaaatcctttgacaggcattttgaaaaaaacagaaacaatgcattagattttgtcaccaaatcctataatgaaacaaaaattaagtttgataagtacaaagctgaaaaatctcacgacgagctccccaggacctttcaaattcctggatacactgttccagttgtcaatgttgaagtgtctccattcaccatagagatgtcggcattcggctatgtgttcccaaaagcagtcagcatgcctagtttctccatcctaggttctgacgtccgtgtgccttcatacacattaatcctgccatcattagagctgccagtccttcatgtccctagaaatctcaagctttctcttccagatttcaaggaattgtgtaccataagccatatttttattcctgccatgggcaatattacctatgatttctcctttaaatcaagtgtcatcacactgaataccaatgctgaactttttaaccagtcagatattgttgctcatctcctttcttcatcttcatctgtcattgatgcactgcagtacaaattagagggcaccacaagattgacaagaaaaaggggattgaagttagccacagctctgtctctgagcaacaaatttgtggagggtagtcataacagtactgtgagcttaaccacgaaaaatatggaagtgtcagtggcaacaaccacaaaagcccaaattccaattttgagaatgaatttcaagcaagaacttaatggaaataccaagtcaaaacctactgtctcttcctccatggaatttaagtatgatttcaattcttcaatgctgtactctaccgctaaaggagcagttgaccacaagcttagcttggaaagcctcacctcttacttttccattgagtcatctaccaaaggagatgtcaagggttcggttctttctcgggaatattcaggaactattgctagtgaggccaacacttacttgaattccaagagcacacggtcttcagtgaagctgcagggcacttccaaaattgatgatatctggaaccttgaagtaaaagaaaattttgctggagaagccacactccaacgcatatattccctctgggagcacagtacgaaaaaccacttacagctagagggcctctttttcaccaacggagaacatacaagcaaagccaccctggaactctctccatggcaaatgtcagctcttgttcaggtccatgcaagtcagcccagttccttccatgatttccctgaccttggccaggaagtggccctgaatgctaacactaagaaccagaagatcagatggaaaaatgaagtccggattcattctgggtctttccagagccaggtcgagctttccaatgaccaagaaaaggcacaccttgacattgcaggatccttagaaggacacctaaggttcctcaaaaatatcatcctaccagtctatgacaagagcttatgggatttcctaaagctggatgtaaccaccagcattggtaggagacagcatcttcgtgtttcaactgcctttgtgtacaccaaaaaccccaatggctattcattctccatccctgtaaaagttttggctgataaattcattattcctgggctgaaactaaatgatctaaattcagttcttgtcatgcctacgttccatgtcccatttacagatcttcaggttccatcgtgcaaacttgacttcagagaaatacaaatctataagaagctgagaacttcatcatttgccctcaacctaccaacactccccgaggtaaaattccctgaagttgatgtgttaacaaaatattctcaaccagaagactccttgattcccttttttgagataaccgtgcctgaatctcagttaactgtgtcccagttcacgcttccaaaaagtgtttcagatggcattgctgctttggatctaaatgcagtagccaacaagatcgcagactttgagttgcccaccatcatcgtgcctgagcagaccattgagattccctccattaagttctctgtacctgctggaattgtcattccttcctttcaagcactgactgcacgctttgaggtagactctcccgtgtataatgccacttggagtgccagtttgaaaaacaaagcagattatgttgaaacagtcctggattccacatgcagctcaaccgtacagttcctagaatatgaactaaatggtaagaaatatcctgcctcctctcctagatactgtatattttcaatgagagttatgagtaaataattatgtatttagttgtgagtagatgtacaattactcaatgtcacaaaattttaagtaagaaaagagatacatgtataccctacacgtaaaaaccaaactgtagaaaatctagtgtcattcaagacaaacagctttaaagaaaatggatttttctgtaattattttaggactaacaatgtcttttaactatttattttaaaataagtgtgagctgtacattgcatattttaaacacaagtgaaatatctggttaggatagaattctcccagttttcacaatgaaaacatcaacgtcctactgttatgaatctaataaaatacaaaatctctcctatacagttttgggaacacacaaaatcgaagatggtacgttagcctctaagactaaaggaacatttgcacaccgtgacttcagtgcagaatatgaagaagatggcaaatatgaaggacttcagtatggagcttttattgaattgaaaccttataccttttgaaaactcattgtgattttcttcatctccatacccctttcgtgatagctcatctgtttttctgctttcagggaatgggaaggaaaagcgcacctcaatatcaaaagcccagcgttcaccgatctccatctgcgctaccagaaagacaagaaaggcatctccacctcagcagcctccccagccgtaggcaccgtgggcatggatatggatgaagatgacgacttttctaaatggaacttctactacagccctcaggtaaataccacctaatgagtgacacgcccccaagagcgagtggagaattggggcagatacatttaattcaggaccaaatattcagagattccccaaactaggtgaaagacaggcggtaagcaacttcttctctgaggaaatattctctagaaagtattacaatgagtccttgattgattttaatgtttagatgcacacatgacatcccatcagcactattatttattaattctgggcaaatccaggaagatgagggttatacctcatcatctaaatcataggcaagctcagccataggcagggtatatttttcagagaggactggtttctgtagtatttaaaactttaaaattcttccccacaatagaattgctagatgagatacatcaaattcctctcatgtcatttacaagctctgccagggccaaatcaagggtgacattaccagaggagaagaccaaacatggttctatgactgttactaaaagtttgtcatgggcttggagaatgcgtactgatgttgggattctgggtctctgcagggtgggctccaacttgccttttttgctatttcttcttttcctatctgtcatttcctgactcttcttctctctcctcttctttctcttccccccactcctcttccagttttcagtcctaggaaggctttaattttaagtgtcacaatgtaaatgacaaacagcaagcgtttttgttaaatcctttctggggcatgtgataaagagaaattaacaacagtagacttatttaaccataaaacaaacacatgaactgacatatgaaagataaatccctttcagtatatgaaagattctctgatctttatttttaactgctaatgaagttttagtgtactatattgtgtaattggagtaattgaaaacatgttatttttttttttctctctgtttagtcctctccagataaaaaactcaccatattcaaaactgagttgagggtccgggaatctgatgaggaaactcagatcaaagttaattgggaagaagaggcagcttctggcttgctaacctctctgaaagacaacgtgcccaaggccacaggggtcctttatgattatgtcaacaagtaccactgggaacacacagggctcaccctgagagaagtgtcttcaaagctgagaagaaatctgcagaacaatgctgagtgggtttatcaaggggccattaggcaaattgatgatatcgacgtgaggttccagaaagcagccagtggcaccactgggacctaccaagagtggaaggacaaggcccagaatctgtaccaggaactgttgactcaggaaggccaagccagtttccagggactcaaggataacgtgtttgatggcttggtacgagttactcaagaattccatatgaaagtcaagcatctgattgactcactcattgattttctgaacttccccagattccagtttccggggaaacctgggatatacactagggaggaactttgcactatgttcataagggaggtagggacggtactgtcccaggtatattcgaaagtccataatggttcagaaatactgttttcctatttccaagacctagtgattacacttcctttcgagttaaggaaacataaactaatagatgtaatctcgatgtatagggaactgttgaaagatttatcaaaagaagcccaagaggtatttaaagccattcagtctctcaagaccacagaggtgctacgtaatcttcaggaccttttacaattcattttccaactaatagaagataacattaaacagctgaaagagatgaaatttacttatcttattaattatatccaagatgagatcaacacaatcttcagtgattatatcccatatgtttttaaattgttgaaagaaaacctatgccttaatcttcataagttcaatgaatttattcaaaacgagcttcaggaagcttctcaagagttacagcagatccatcaatacattatggcccttcgtgaagaatattttgatccaagtatagttggctggacagtgaaatattatgaacttgaagaaaagatagtcagtctgatcaagaacctgttagttgctcttaaggacttccattctgaatatattgtcagtgcctctaactttacttcccaactctcaagtcaagttgagcaatttctgcacagaaatattcaggaatatcttagcatccttaccgatccagatggaaaagggaaagagaagattgcagagctttctgccactgctcaggaaataattaaaagccaggccattgcgacgaagaaaataatttctgattaccaccagcagtttagatataaactgcaagatttttcagaccaactctctgattactatgaaaaatttattgctgaatccaaaagattgattgacctgtccattcaaaactaccacacatttctgatatacatcacggagttactgaaaaagctgcaatcaaccacagtcatgaacccctacatgaagcttgctccaggagaacttactatcatcctctaattttttaaaagaaatcttcatttattcttcttttccaattgaactttcacatagcacagaaaaaattcaaactgcctatattgataaaaccatacagtgagccagccttgcagtaggcagtagactataagcagaagcacatatgaactggacctgcaccaaagctggcaccagggctcggaaggtctctgaactcagaaggatggcattttttgcaagttaaagaaaatcaggatctgagttattttgctaaacttgggggaggaggaacaaataaatggagtctttattgtgtatcata

SEQ ID NO: 2 - DNA, 1870 bp, Homo sapiens
agagactgcgagaaggaggtgcgtcctgctgcctgccccggtcactctggctccccagctcaaggttcaggccttgccccaggccgggcctctgggtacctgaggtcttctcccgctctgtgcccttctcctcacctggctgcaatgagtgggggagcacggggcttctgcatgctgaaggcaccccactcagccaggcccttcttctcctccaggtcccccacggcccttcaggatgaaagctgcggtgctgaccttggccgtgctcttcctgacgggtaggtgtcccctaacctagggagccaaccatcggggggctttctccctaaatccccgtggcccaccctcctgggcagaggcagcaggtttctcactggccccctctcccccacctccaagcttggcctttcggctcagatctcagcccacagctggcctgatctgggtctcccctcccaccctcagggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaagtaaggacccagcctggggttgagggcaggggtagggggcagaggcctgtgggatgatgttgaagccagactggccgagtcctcacctaatatctgatgagctgggccccacagatggtctggatggagaaactggaatgggatctccaggcagggtcacagcccatgtcccctgcaaaggacagaccagggctgcccgatgcgtgatcacagagccacattgtgcctgcaagtgtagcaagcccctttcccttcttcaccacctcctctgctcctgcccagcaagactgtgggctgtcttcggagaggagaatgcgctggaggcatagaagcgaggtccttcaagggcccactttggagaccaacgtaactgggcactagtcccagctctgtctcctttttagctcctctctgtgcctcggtccagctgcacaacggggcatggcctggcggggcaggggtgttggttgagagtgtactggaaatgctaggccactgcacctccgcggacaggtgtcacccagggctcacccctgataggctggggcgctgggaggccagccctcaacccttctgtctcaccctccagcctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 3 - DNA, 357 bp, Homo sapiens
acagacaccaaggacagagacgctggctaggccgccctccccactgttaccaacatgaagctgctcgcagcaactgtgctactcctcaccatctgcagccttgaaggagctttggttcggagacaggcaaaggagccatgtgtggagagcctggtttctcagtacttccagaccgtgactgactatggcaaggacctgatggagaaggtcaagagcccagagcttcaggccgaggccaagtcttactttgaaaagtcaaaggagcagctgacacccctgatcaagaaggctggaacggaactggttaacttcttgagctatttcgtggaacttggaacacagcctgccacccagtga

SEQ ID NO: 4 - DNA, 3482 bp, Homo sapiens
gtcatggttacctccccacctccccagctgcaaggaggattcatccggcaaccagttgaggctagattctcagcagctttattgaatattgagaggtggtctcacctcccactggacatgtgtcctcaagttcataccagaacttctttgggacagacagacagacaggtggcagggcagggcaggtgtccacgagggtggggccagtgcaccaggggcagctcagctctccaaaggggccagcatctgcacctgctcctgctgctgctcctgctgctgttcctgctgttgctccagctcagggagggagagagtcttgtcctggctctctttctccttgaaggtgctgaagaaggagttgaccttgtccctcaggtccttctccaggaagctcaagtggccttccacgtcccccgcatgggggcccagtttctgcctgagctgttccatctgctgcaccagggctttgttgaagttttccccgtagggctccacccggcgtcggaactcctccacctgctggtccaggtgcccacccagctctgccagtgacttctgcagcccctcggtgttgcccctcaggttgccacgcacgtcctcggccaagggcgccagcctctgccgcagctcctcggcactggccgagatcctggccttgagctcctcggcgttcttcttcatctggaaggtcaggccctcaagctggtggttgagcttctcctgcgtgtcctgagcatagggagccaggctgcggcgcagctcctccacggtctggtcaatcttgactttgaattcgtcagcgtagggcgtaaggcgtcccttgagctcctccacgttctggtcgatcttggccttgagctcgtcggcgtggggcctcagcgaggcctgcaggctgtcggcgttctcccgcagcactctctccatgcgctgtgcgtagggggtcagctggcgccgcagctgctcggcctgcgtgttgacctgggtgcgcagctggtccgcgtagggctccaggcgctgctgaagctctcgcaggttgtccccgatcttctggctcacctcattggcatggggcagcagccgggccctcagctcctccagctccttcccaatctcctccttcagtttctccgagtccttggccaggcgttcatgcagctcggtggcaaagggcaccagcttcttctgcaggtcacctgcgtaagtgttcacttctccaagtttgtcctggaagagggcactgtggggaagggcacaaggaggccacgttacatttggcatttacacggcaagactttgtctgcttgtataccactcgccttatgcacacgtgctaagacaggtgagtgctcagaggtaccgagttcaccctccctatggtggtgcagattggagaggatgggtgtcacagactaagttacgatgctgaatttctatctcaggatctcccacatagtttgtctcagaatctacccaccatgtcacctccagcagtttgctttcccctccttgtctgcagagggtgggactttgaggtccttcccagagggagtgttctgggccataggatcacattgtgtaacacaatgttgtcttcttgacctctgctggggtcttccagtggcacaaggtgctgctttccttgcctgtgcccaggggcctcccaaatattcccacctccctccccttgattccactggggcctgtctttctgaaacgtattagaaatcagctcacatgacattttcctccctccctctcttccatgagcctgaatgataggatgtggccatttgtcaaactctagaacgtcaaagcaaaaggacatggcagggattattcggtccaaacttccggtttacatatgtggatcctaggttcagaggcccggccagttagtggcagggcagtgggtatcctaagctcagggctcctgtctctaagctcaacccttgccagtacattgcatggcctttaagaattccccgtcaccaccgcacactgtagtccctcttacttgagttgctgggtgagttcagatttctggagatgttccacggcctccttggcattgttgctcagctggctgaagtagtcccacatcactgtggccacctggtcagcactgacctcagccctggctcctgggtggtaacagagagcaattcatgaggccccgtctcctgtgtggcccctctgctccagctctgagctgcagactggatgatggtgggggtgcctcaacctgccattttccctgtctgagcttagctttttggaagccacagggatatgtgaaactggtatagcaccaatgccaatgggcccaccactgggactgggccagctctcctgtgagccacttggcagccaggcagacctcatgtccagctgggggctgatgggcactcagaagtgtccttttgctttggctcagcctccatcctgcactactcagagcagcagcccaggagtgccatccaaagacagcttctactcaccggcgacagccaccagggccagggtcaggaccacggccttcaggaacatcctgagctgcttgctgggctggaggagtttcttgccacactggatcctccctacaatcaggggagctgacagagaggtcctcaggagagctcacctgcgctgcagtgggaactgactgaagctcagagccagccagacatttaaactctctccctatcgccacccccctggctgccctcccctcctccttcctcagtgtgactccacgctggaaggtgacacattcccaagaggcctcttggacttttgtgaccctgagactacgtggaagctgacagcagatcagggctcaggcgatagttagaagtggtggctgttccgtgcgtgggcacccccacccccaccccaccaacctcagcatggaagggaggaggggaacggaaaacagtaggagaaacacctcagggagcccatgagcccgggactgatgcctcgaggccctgctggcggctctgggtttgtgccgggctcatggggggcaaagtccacatctcctctgtctaccccagtgaagacaggtccccccagtctccacatccgccactaatgctgactccatctcagagcaccagacactggagtgggaccatccaggactgcagacagcatagtgagctaaaatctaggtctcttgtcaaggtacacccaagaggagggggccctggagagcctttgggcagatgggtgtgggagacagggccttgtcctgaaagggttaattgtagagtgcttcatgtcccactcccatgctgtgcctccttggtctagttattcaaagccctgagcttcagtttctcctctgaacaatgggatggttcagtacattaaagaagacattgcttgcgaagtgggtagtacggtcatctctcagtctccatgggaaattgattccaggaccctctgcagataccaaaatccatggatgttcaagtcct

SEQ ID NO: 5 - DNA, 1195 bp, Homo sapiens
ccaggcctccctccacctgtcttctcagagcagataatggcaagcatggctgccgtgctcacctgggctctggctcttctttcagcgttttcggccacccaggcacggaaaggcttctgggactacttcagccagaccagcggggacaaaggcagggtggagcagatccatcagcagaagatggctcgcgagcccgcgaccctgaaagacagccttgagcaagacctcaacaatatgaacaagttcctggaaaagctgaggcctctgagtgggagcgaggctcctcggctcccacaggacccggtgggcatgcggcggcagctgcaggaggagttggaggaggtgaaggctcgcctccagccctacatggcagaggcgcacgagctggtgggctggaatttggagggcttgcggcagcaactgaagccctacacgatggatctgatggagcaggtggccctgcgcgtgcaggagctgcaggagcagttgcgcgtggtgggggaagacaccaaggcccagttgctggggggcgtggacgaggcttgggctttgctgcagggactgcagagccgcgtggtgcaccacaccggccgcttcaaagagctcttccacccatacgccgagagcctggtgagcggcatcgggcgccacgtgcaggagctgcaccgcagtgtggctccgcacgcccccgccagccccgcgcgcctcagtcgctgcgtgcaggtgctctcccggaagctcacgctcaaggccaaggccctgcacgcacgcatccagcagaacctggaccagctgcgcgaagagctcagcagagcctttgcaggcactgggactgaggaaggggccggcccggacccccagatgctctccgaggaggtgcgccagcgacttcaggctttccgccaggacacctacctgcagatagctgccttcactcgcgccatcgaccaggagactgaggaggtccagcagcagctggcgccacctccaccaggccacagtgccttcgccccagagtttcaacaaacagacagtggcaaggttctgagcaagctgcaggcccgtctggatgacctgtgggaagacatcactcacagccttcatgaccagggccacagccatctgggggacccctgaggatctacctgcccaggcccattcccagcttcttgtctggggagccttggctctgagc

SEQ ID NO: 6 - DNA, 8690 bp, Homo sapiens
ctgggaatacaggcgtgagccactgcaaccagccagtagcccccatctttgcccctcgctgagccctactggatgttcttggttatgcgacagtttccccatctattaaagagaaacccctatagcagaggggaggatgaggttggaaaagcaggagcattgttatgctattcttgtggggtctgggaagcagacatctgggtggatgtttggggggtgctgggcttagttggggaagtaggggggcccctggggctgacagggactggaagctctgagctggccagagggatgttgcaatcctgccagggtcttgtctatgctgtccttttcacaaccatccccctactgccaggctgacacgtggttgcgggggcacaaggccagccaacctagagtctgaggctaggcggaggacaccctccccaccagctgccagggtcactggcggtcaaaggcagctggtggggaaggcattggactccagccttgggggacggatgtagtgatggtgggaagcaggcttggtgccaggaggggcgtcagagggtgaataagagcagatagagtgtttgggggaggtagccagccaaagggggtgaggcccggtggaagggaagaagggacatacacgcagagctttgcagctgagggttttaattttttgagatggggtctctgtcccaccaggctggagtgcagtggcacaatcacagctcactgcagcctcgaactcctgggctcaagcaatcttcctacctcagcctcttgagtagctgggactacaggcatgcgccaccacgctcggctaatttttgaacttttttgtagagatgaggtctccctatattgcccaggctggtctcgaactcccaggctcaagtgatcctccctcttcagcttcccaaagtgctgggattacaggcatgagccaccatgcctggctaatgcaggtgaggtttttgcagtgtcatccagctaaggcgacccgttcccctcccaaaaaagggagactgagaaccatgaagttaagagcccagagaatatcacggtggtctggggtgcttcaagggctggtctggaataaattggaggtggcacgcagggtaggagcgccgggccaactgggagacccagcaacataaaggaaaagttgttggggctgaggaggcttgctgagagaggggaagtgagggaaagaggtgatctagggacacggtgtgaatgagggggggatgagatcacagggttattactgggagacccctgagggaagatggccacagggacaggacaaggctgtcttcttaagggaggagaccacccctcatattgtcttatgcccaatttctgcctccaaagaaagaaaaagtaaaaactaaaaggcagaaatgaaatccacaagcagacagcccgcgccacaccctgggcctggtggttaaagattgacccctgacctaatccgttaggttatctatagattacagacattgtatagaaaagcactgtgaaaatccctattctgttttgttccgatctaattaccggtgcatgcagcccccagtcacgcatcccctgcttgttcaatcgatcacgaccctctcacgtgcacccacttagagttgtgagcccttaaaaggaacagggattgctcactcggggagctcggctcttgagacaggaatcttgcccattccccgaacgaataaaccccttccttcgttaactcagcgtctgaggaattttgtctgcggctcctcctgctacattctgagtggggaaagggactaaggtggtctgaggaccccacagagtcaggaagattgagaggtgagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctgagtgcctgggagggacacccgcctacactctgcaagaaactcaaaaagggagatgaggggatcgtgggagggaggtagggagggaggagggtgccactgatcccctgaacccctgcctctgcctccagagtgcccctccggcctcgccatgaggctcttcctgtcgctcccggtcctggtggtggttctgtcgatcgtcttggaaggtaaaagtgggatgggagaattgcggagttggagatttggaagagtgaaggtggctacaggcctggggtcccggcttagaggacctctgagagctccggggccccttctgggtcgtggttgcctcatcgtggtcgggtgggtctccaggttctcccaggctcagtcccgcaggcgccaaatctgcgcaggagagcactagcaaccgatgacgtattgaggcccacacctctgggattggctgtcctgcttcgacagccttgaaagtgggtaagctgggtggggggctctgggagaggtcagtgctgagtaaggcaattcccagcagcttgagccccaccaggtcactccagtattcctccccattctttttttttttttttttttttctcttgagacggagtctcgctctgtcgccgaggctggagtgcagtggcgcgatctcggctcactgcaagctccgcctccctggttcacgccattctcctgcctcagcaggactacaggcgcccgccaccgcgcccggctaattttttgtattttcagtagagacagggtttcaccgtggtctcgatctcctgactttgtgatccgcctgcctcgacctcccaaagtgctgggattacaggcgtgagccaccgcgtccggccattcctccccattctaaccacatgatccccaaggatctctatccatcccggtatcccaacctaagggggttccaataacaaatttttggccgggcagggtggctcatgcctgtaatcccagcactttgggaggccgaggcgggcagatcacttgaggtcaggagttcgaaaccagcctggccaacatggtgaaacttcgtctctactaaaaatacaaaaaaattagccaggtgtggaggcacgcgcctgtaggcccagctactcgggaggctgaggcaggagaatcacttgaacccgggaggcggaggttgcagtgagccgagatcataccactgcactccagcctggctgacacagcaagactccgtctcaaaacaaaacaaaacaaaaatagctgggtgtggtggtgcacacctgtaatcccagctacttgggaggctgaggcaggagaactgcttgaacccgggaggtggtggttgcagtaggccgagatcatgccactgcactccagcttgggctacagagcaagactccatctccaaaaaaaaaaaaaaaaaaacaaattttgaacccctgcccatcttcctggcaggcccagccccagcccaggggaccccagacgtctccagtgccttggataagctgaaggagtttggaaacacactggaggacaaggctcgggaactcatcagccgcatcaaacagagtgaactttctgccaagatgcggttagaacccttcccagggcacgggagagctggggtgtgtttttgggtggagccctggcagatggtccaagatgaacagattgaaaaaaaaacaagtcctggagaggctgacaacatccctctggtcacacagctagatctcaaggtgctcagacttcaaggacagtttccctgactcccatccaggccatattttaaaagatggtcttgggctgggcacggtggctcatgcttgcaatcccagcacttagggaggccgaggtgggctgattgcctgaggtcaggagttcgagaccagtctgaccaacatggtgaaaccttgtctctactaaaaatacaaaaaaattaggcaggcatggtggcgtgcacctgtaatcccagctagtcgggaggctgaggcaggggaattgcttgaaccaggaaggtgggagttacagtgagccaacattgtgccagcctgggtgacagaaggagactctgtctcaaaaaaaaaaaaaaaaaaaaaaaaaacaagatggtcttgcccaggtatggtggctcacacctgtaattccagcactatgggaggctgagatgggaggattgcttgagcccaggagttcgagaccagcctgaccaacatggcgagatcctgtctccatttaaaaaaaaaaaaaaaaagatggttttgtgaggtaatgaaaatgaaggccccaagcttggccagacctgggtccccaggctggagtagcaccccttcctgtgtgatcttgacagaggggcattactgtgagcctcagtttcctctcctataaactggtggttctacagggaagtaaaggagcaggcctacagggtgtctggtacatgtagatgctcagtatatcatgaaacccacccttgccccctttggcaagttagagagtcattcgttctttcaaaaatatttactgagcatctgctaagtgctggaaactgtttcaatgtggggaataaaacagtgaagaacgtgccgagcacggtggctcacacctgtaaccccaccactttggaaggccgaggtgggtggatcacttgaggtcaggagtgcgagaaccccgtccctaatagaaatgcaaaaaaaattagctgggcatggtggcccatgcctgtagtcccagctccttgggaggctgaggcgagaggattgcttgagcccaggagatctaggctgcagtgcgccatgtttgtgccactgcattccagcctgggtaacagaatgagaccctgtctcaacaaaaaaagaaaagaaaagagaagaaaagagaaaagaaagacagggagggagggaggaaggaagggagggagggagggaaaatagagccaggcataaacttagaaagatcgtttggaggccaggcacaatggctcacacctgtaatcccagcactttgggaggccaaggcaagcagatcacctgaggtcaggagttcgagaccagcctaacatggagaaaccctgtctctactaaaaatacaaaattagccgggcgtggtggtgcattcctgtagtcctagctactcgggagcctgaggcaggagaatcacttgaacccgggaggcggaggttgcagtgagccgagatcatgccactgcactccagcctgggcgacaaggcgagactccatgccaaaaaagaaaaaaaactcctggcgcggtggctcacgccagtaatcccagcactgtgggaggctgagcaggcggatcacgaggtcaggagttcgagactagcctgctcaacataatgaaaccctctctgtactaaaaatacaaaaattagctgggtgtggtggcaggcacctgtagtcccagctactcgggaggctgaggcaggagaatggcttgaacctgggaggcagaggttgcagtgagccgagacagtgccattgcactccagtccaggtgacagagcgaaactccatctcaaaaaaaaaaggaaggcattggtagcaagagatggcaggccttgaaagccaggccagggtgaagtgtttcttttttttttttttttttttttctttttaaatttttttttttgagacggagtctcgctctgtcacccaggctggattgcagtggcctgatctcggctcactgcaagttccgcctcccgggttcatgccattctcctgcctcaccctcccgagtagctgggactacaggcacctgccaccacgccagctaattttttgtattcttagtagaatgtagaatttacttagtagaattttttgtattcttagccagcatggtctcgatctcctgacctggtgatccacccgcctcggcctcccaaagtgctgggattacaggcgtgagccacggcgcccggccttattttttctttttgagatgtacccagactggagtacagtggtgcgatctcggcttactggaacctccacctcccgggttcaggcaattctcctgcctcagcctcatgagtacttggaactacaggtgtgtgacaccacacatggtattttttgtatttttagtgaagatgacatttcaccatgttgcccaggttggtctcgaactcctgacctcaagtgatcagcctacctcggcctcccaaagtgttgggattacaggcgtgagccaaatgcccagccaagggtaaagtgtttagacttcaacgtgctttggtccatctgggaaactgaggcacagaagttggcccacccagcccagcggtcctcctaatcccacagacagtggggatggagattctgcaaggggaagaggtgggagtcaggtagcaggcagaatttggacagcctgggaggtagctgcacacagtgacccccttccttattcctccccacagggagtggttttcagagacatttcagaaagtgaaggagaaactcaagattgactcatgaggacctgaagggtgacatcccaggaggggcctctgaaatttcccacaccccagcgcctgtgctgaggactccctccatgtggccccaggtgccaccaataaaaatcctacagaaaattctctcctgagtgcttctttactctggggaaggggctgcgggagagggtaggggcttccagagagggcagggtctgcaggagagggcaggggctaaaccttaggtactcctcacaagccctccaatgccctatctacttgccctgtgctgaggatgttttaactccatggtctcaaaagagtcttcctaagaaccctgcaaactgggccttattaatcccataagggcattgaggcccagagaggtgaagttacttgtataaggtcacacagccaggaagtagagaactggaactagattgaaccctcagcctagcaatgtcactatgctacacttttcctagtgtggtctacccgagatgaggggctgaggtttttttttgtttttgtttctgttttgaggcagactcactctctcccccaggatggagtacagtggtgcgatctcagctcactgcaacctccacctcccaggttcaagagattatcctgcctcagcctcccaagtagctgggatttacaggtgtgcgccaccacacccagctaatttttgtatttttagtagagacagggtttcatcatgttggccaggctggtctccaactcctgggcttaagcaatcctcctgccttggcctcccaaagtattagaattacaggcgtgagccactgtgcctggctcttatgtaaaattaaaccacatacacatgagaaacaaccctatgtaattaagatttctttctttttttttttttttttaagagatggagtcacccaggctggagtgcagttgcacaatctccattcactgcagccttgcaacctccaccacctgagttcaagggattctcctgcctcagcctcctgagtagctgggattataggcatgtgccaccacgcccagctaatttttttgtatttttagtagagacggaatttccccatgttggccaggctggtctcaaactcctggccttaaatgatccacccgtcctggcctcccaaagtgctgggattacaggtgtgagccaccgcacccagcttaagatttcataagaaaaatatttgtaagccacatatctaataaccggttaatattcaggctgggtgaagtggctcatgcctgtaatcccagcactttgggaagacaaggcgggcggattacctgaagtcaaaagtttgagacctgcctggccaacatggtgaaaccctgtctctactaaaaatacaaaaaaattagccgggtggggtggcacatgcctgtaatcccagctgcttgggaggctgaggcaagagaatcccttgaacccaggagtcgaaggttgcagtgagccaagatggcgccactgccctccagcctgggagacagagtgggactccatctcaaaaaaaaaaaaaattcaaaatgtataatatacaatcctagttgggacatcaaacactgcagccactgtggaaaacagtatggggtttcctcaaaacattaaagatagaactctcaaatgatccttcaatcccacttctgggtatttattcaaaagaattgaaatcaggaccttgaagagatacctgccctcccatgttcactgcaggtctgctcaatacccaagatatggaaacaacctaaatgtttatcaacagatgaatgggtcaaggaaatgtggtctctacatgtaatggaatgtaacgcatccttaaaaaggaaaccctaaggccgggctctgtggctcacacatgtttttttttttaaattttatttatttatttatttatttatttattttttattgatcattcttgggtgtttctcacagagggggatttggcagggtcataggacaatagtggagggaaggtcagcagataaacaagtgaacaaaggtctctggttttcctaggcagaggaccctgaggccttccgcagtgtttgtgtccctgggtacttgagattagggagtggtgacgactcttaacgagcatgctgccttcaagcatctgtttaac

SEQ ID NO: 7 - DNA, 7576 bp, Homo sapiens
aaagaggaactgacagcaaggctaagccaatgtctgcccctgggggcagaaagtcacctcctgctctccctccactgtccacagaggtagctcagacagggtgggggtcacaggagaacgaagggagaagggggtagttcctgggcagcaaaatcaggtggtgaagggaggcatcagaggatggcaattagagaggccattagaggggaaccacaggcagacagggtgacaggagggactactgacacaaggtgaagagatggcccagccggacggggtggctcacatctgtaatcccagcattttgggagcccgaggtgggtggatcacttgaggtcaggagttcgaggccccaacatggcaaaaccccatctcttctaaaaatacaaaaattagccgggcatgatggcagatgcctgtaatccctgctactcgggaggctgaggcaggaaaattgcctgaatccaggaggtggaggttgcaatgagacgagatcatgacactgcactccaccctgggcaacagagcaagagactgactctgtctcataaaaaaaaagaaaaaagaaaaaaaaaaagagatggctgatggttaaagaggggttagcggtcaggggacacataagggtaaaggcaggaggcaagaggactggcagggggctgcccctgggccaccgggagcgacacaggatgagcatggagggaaagggagaaggggattctagggtcccagcctacccaagttgccctctggttccacctagcatgccagccagaggcccaggaaggaaccctgagccccccaccaaagctaaagatgagtcgctggagcctggtgaggggcaggatgaaggagctgctggagacagtggtgaacaggaccagagacgggtggcaatggttctggtgagggtgtgctgggctgggtggtgggaggggactcctgggtctgagggaggaggggctggggcctggacccctgagtctcagggaggaggaaagggtgggagtggggctgtgacccctaggtctgggaggagtggagggttagagctgagagcaggaactcctaggtcacagagaggagcggataaatggggcagagaacacctggggagagctggggcctccactgtgatgtcctctctcctgtaggagcccgagcaccttccggggcttcatgcagacctactatgacgaccacctgagggacctgggtccgctcaccaaggcctggttcctcgaatccaaagacagcctcttgaagaagacccacagcctgtgccccaggcttgtctgtggggacaaggaccagggttaaaatgttcataaaagccaggtgtggttgtggcgggtgcctgtagtcccagctactcaggaggctgaggtaggatgatggcttgagcccaggagttcgagaccagcctgggcaacacagcgagatctcttgggggtaaaacaaaaagaaaaaaaaaagttcatacttctccaataaataaagtctcacctgtgtccctgtctggatccttccccagtgtggccagaaaaaaacccaccccactgcctcccaggaatcaatgagtagaagaggtgacacctgatggggaaggaagagtagggaggtcgggaagggtatcaaggaataacaccctattgtgggcttgcggagaatgggggacttcaaggcgtgtcagtttcaggagggtgagggcaggagcgtgggtggagtcagcaggtccccatgatggccctcactgagagcttcgcccttgtctcctacaagctctgactccattcccagtgggcacccagcacctccaacccctccacagcccccaacccagcctctgtcggaggcgaattctcagagtgagggttccctgtcacttgagagaaggttccctgtgacgtgaccttgggggacgtcattgccctttctgtccccacccaccccctccgcagttctgttggccaggactttggcctagacaaaggatgggggttgtggctgtggagcggaagtgggtctcaaccactataaatcctctctgtgcccgtccggagctggtgaggacagcctgccagagtctggtaagaaagggactcagggtgcggggacaggggggcgtcagcagggagagggcaaagatcgataaagcaggaattttaagaggcacaatattagaagcccgtgttggaaccatgactgtgtgtgtgtgtgtgtgtgtgtgtgtgtgtgtgtgtgtgtgtgagagagagagagagggagatggagtctcgctatgtagcccaggctagactcaaactcctgggctcaagcaatcctcctgcctcagcctccccagtagctgggactacaggtgcaccaccacactccacaaatcacagaatttagaactgtagactatttgagcttctgcttagagttagggtggctgaggtggggaggatcccttgagcccaggagtttgaggatgcagtgagctgtgatcttgccaccgtgttccagcctgggtgacagagaaaccccatttctaaaaaagagaaagaaaaagggataggtacaatggctcatgcctgtaatcccagcactctgggaggccgaggcgggtggatcacttgaggtcaggagttcgacaccagccttaccagcatggtgaaaccgcatctatactaaaaatacaaaaattggccgggtgtggtagcatatgcctgtaatcccagctattccagaggctgagacaggagaattgcttgaacccaggaagcggaggttgcagtgagcccagatcgtgccactgtactctagcctgggtgacagagcaagactcagtcttggcggaaaaaaagaatgaaaaaatttaaaaaactaaaaaagaactgtaggctgggcgtggtggcttacacttgtaatccaaacgctttgggaggccaaggcaaacggatcacttgatgtcaggagttggagaccagcctggccaacatggtgaaaccccgtctctactaaaaatacaaaaattagacaggcatggtggtgcatgcttgtatttccagttactcaggaggctgaggcaggagaatcgctcgaacccggaagacagaggttgcggtgagccaaaattgcgccatcgcactccagcctgggcgagagaacaagaccttgtcttggaaaaaaaaaaagaattgtagaccatttgcttgtgttctttctccggggatcagatctcaccctctttctgccgtacttcctcatctcctacgtgtggatgatgatattgtgccctgtgcatgttcttcgtcaccaaaagtgcctctctcatagagcaggtgagaactcagtgaggagatgcagggacatgaggtctgacttagggcagagccctaaggtaacacatttgatctactgtaggtccttaatggtgtctgcagagcacctccctgcactgactcagccttagcaaagggcagaggctttgctgtgttccctgctgggcccagaactgtttaggtgctcaagaaagccttctaggctgggctcagtggctcacacctgtactcccagcaccctggggaggccgagatgggaggatcgcttgagcccaggagttccagaccagcctgggcaacaaaacaagtctcccatctctacaaaagaataaaaattagcagctgggcatggtggctcatgcctgtaattccagcactttgggaggccaaggcaggcaaatcacttgaggttaggagttcaagaccagcctggccaacatggtgaaaccccatctctactaaaaatacaaaaatcaggtggggcacagtggctcaagcctgtaatcctagcactttgggaggccaaggtgggcggatcacgaggtcagaagttcgagaccagcctggccagcatggtgaaaccccatctctactaaaaatacaaaatattagccgggcatggtggcaggtgcttgtgattccagctccttgggaggctgaggcagaagaattgctagaaccctggaggcagaggttgcagtgagccgagaacacgccactgcactccagcctgggtgacagagcgagactccatctcaaaaaatacgaaaacaaaaatcagccgggtggtggcgggtgcctgtaatcccagctactggggaggctgaggcaggagaattgcttgaacctgggaggtgggggttgcagtgagccaagattgcaccactgcactccagcctgggcaacagagtgagattccatctcaaaaaagaaaaaaataataattaaaatgttaaaatcaggagtagaatcacagaatgttggaaagtgaggcccaagaagggggctgtgtccaagtccatgcatgggaaacttgactgggacaccgagctcacacagagcaggatctcagtcccccccaccagagtggggcgtgaccacaggaacagccgcctccagtcagcctgccacatgacaccccctcaatgttccaggtctctggacactatgggcacacgactcctcccagctctgtttcttgtcctcctggtattgggatttggtgagtgtgggcttccggggagggaagccttggggaggggaatgagctccaagcatcttcccagcccaggcccttcttacctctgcctctgccctctcctcttcttccttcctcctttccccctgctgcagccccacgggctctcctgacacactctccccctgcagaggtccaggggacccaacagccccagcaagatgagatgcctagcccgaccttcctcacccaggtgaaggaatctctctccagttactgggagtcagcaaagacagccgcccagaacctgtacgagaagacatacctgcccgctgtagatgagaaactcaggtagcacctgcccctggagaaatggggtctggcccataccaccgactgcatccaggacccagaagttcaggccccagcccctcctccctcagacccaggagtccaggcctcagcccctcctccctcagacccaggagtccaggcccccagcccgtcctccctcagacccaggagtccaggcccccagcccctcctccctcagacccaggagtccaggtccccagaccctcctccctcagacccaggagtccaggcccccagcccctcctccctctaaccatctgtgctttctccccagggacttgtacagcaaaagcacagcagccatgagcacttacacaggcatttttactgaccaagttctttctgtgctgaagggagaggagtaacagccagaccccccatcagtggacaaggggagagtcccctactcccctgatcccccaggttcagactgagctcccccttcccagtagctcttgcatcctcctcccaactctagcctgaattcttttcaataaaaaatacaattcaagttgcttctcatggatggcactgcttttctgaggactcaaggtgccaagatggaggggctgactcagtccagccaacatttaatgagcacctactttatgtatggagctctaacccatgggtccatgggaataaagcagtgaatagtaacaataaataatcgtaacagcaattagagactaatctttattgaagtccggctttctctcttttatttttttattttttgagacagggtttcactctgtcacccaggctggacagcagtggtgcaatctcggctcactgcaccctccgcctcccaggttccagcgattctgtgtgcctcagcctctggagtagctgggattatgggcatgtgccactgtgcacggctaatgtttgtatttttagtagggttttgccagttggctaggctggtctcaaacttctgacctcaagtgatccacccgcctcagcctcccaaagtactggggtgacaggcatgagccaccgcgccatgccaaaatccagccttctcatttgtaaaataacaaatcatagacacagctagtccacagtggcctctgacagtctccagttgttaacggtgaatggtgtccaggtttttggcgtcctgaacaaagaactggacaaaacgcacaaacaaagcaaggaaggaatgaagggatttattgaaaacgaacatacactaaacagtgtgggagcgggcccaagcatatgggttcaaaagccctgttacagaatttttgggagtttaaattccccctagaggattccccttccactggttacttctggtacaccctatgtaaatggagaggatgaagaaaagttacaaagtcatttatggcctaccggagaggatgtttcctgttatagctgaagtgaattggccttatgttccctgcctccctattttcctgccttacagttgctggactgacacattttttattttatttattttgttttgagataaggtcttgctctgtcgcccaggctgaagtgcagtggtgagatcttggcttactgtagccttgaccttttgggctcaagagatcctcccatctcagcctcccaagtagttgggactagggatgcccactgccacacctggctaattttttgtatttttagtagagacggggtttcgtcaagttgcctaggctagtctccaattcctgggctcaagcgatctgcccaccttgcccttccaaagtgttgggattacgggtgtgaaccaccgcaccaggcctgggctgacactttaaacgtgccattgcaatcagtgtataatggtcccgtggcagagggagaatggtgcccaagtcagtacttcttggctgggacccaagaagacaaacctctagggccggcgtggtggctcacgcctgtaatcccagcactttggttggctgaggtgggcagatcatctgaggtcaggagttcaagaccagcctggccaacatggtgaaaccttgtctctaataaaaacacaaaaattagctaggggtggcccgcgcctgtaatcccagttacctggaggctgaggcaggagaattgcttgtaccctggaggcggagatagtgccactgcactccagcctgggcagcagagtgagactccgtctctaaaaaaaaaaaagaaaaacttattaaaaaaaaattgcaagcaaaaagcacagaaggcagaaggtgcaaataagcagaggctccatggtctgaaggaggccagtgctgggaccatgaatgaagggggctgggggccaagattcctgtatcctgagggagtccagggtggtactgctccccaagtcccatgggtggggctattggtctctggggatccactgcttgtatttctcctaatctgcttggcattaacctcctgccaggctttcaacacccttggggcagttttcccaggtcctgaggctgggcagctgagtccagtaaaaaacaggggccctggagagggactgtgggcaatgcccccgagtccccggggcctgggaagaacagaggctgccaagtgcagggggtggtggggaggggaagaccctggagtaacaatccctgctgtgtccacttggttcccacggactcaagccaagccaaagcactgcccggaccagttcggcttccaaccaatcctggtgctggcc

SEQ ID NO: 8 - DNA, 514 bp, Homo sapiens
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggctacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgccagctccttgggtcctgcaatctccagggcttcccctgtaggttgcttaaaagggacagtattctcagtgctctcctaccccacctcatgcctggcccccctccaggcatgctggcctcccaataaagc

SEQ ID NO: 9 - DNA, 4510 bp, Homo sapiens
gagacggagtcttgctctttcgcccaggctggactgcagtggcgcgatctcggctcaatgcaagctccacctcccaggttcacgccattctcctgcctcagcctcccgagtagctaggactacaggcgcctgccaccacgcctggctaatttttcatatttttagtagagatggggtttcaccgtgttagccaagatggtctcaatctcctgacctcgtgatccgcctgcctcggcctcccaaagtgttggggttacaggcatgagccaccgcgcctggccaacagcaatgatctttgagcacctatattgccagtctccacggtaagagctttcttcattttttgttttgttttgtttcaagacagagtcttgctctgtcacccaggctggagtgcagtggtgtgatcgcggctcactgcagccttcacttcccgggttcaagccattctcctgcctcagcctcccaagtagctgggattacaggcacgcatcactacttctggctaatttttgtatttttagtagggacagggtttttcaccatgttggccaggttggtctcaaactcctggcctcatatgatctgcccacctcggcctcccaaagtgctgggattacaggcgtgagccactgcgcctttctttgtatttgttcaagtaatatactgaaatatgtactgtgcctcccactttatggaggaggaaactgaggccagcaaatgaggctgtcatgggaggtggagacaggatttgaacctgcctcagtgcaggaggctcaagagcctctgtcttctctcagggcactgtgtgggagggtgagaaggagggaggcccacagaggcatgacctctgattgccactgtcacctgggccctgctctctgaagtctctgccaagcggggaggtggccgggggagggccctgctctgtgcagcctcccctcccccggcccgcagagttgagcacagagggacagaggcacggaacccccagaaatgtccctcctcagaaacaggctccaggccctgcctgccctgtgcctctgcgtgctggtcctggcctgcattgggggtgagaagaagtgggtggagggatgtggggcccacacctggtgggtgtgagtgtggctgtgtgtcctgtggctctgtagccacgtgagacatgagtacggagtgtgtgcgtttcatggcgtgcgtatgcatgtgcgtgtcggggagtgtgtgtgtcggtggctgagagtgaagtgtgaatgtcacattggtacaaactgggatcatctgtgtgtgtgcacgtgcgtgcgtggaagtgggagtatgcagtcgtggtaaaaaagtgcatgtctgtgtgcatatgtgtatttgtgtgcacctgtctctctgtggggtatgtgtgtgcaaaatatttgagtgtgtggacatgtgtgagggggtgagtgtgtgctggtgtgtacgtctgtgttttgcatatgcatttttttttttttttttgagacggagtctcactctgtcacccaggctggagtgcagtggtagcagtggtgcgatcttggctcactgcatcatccgcctacccgtttcaagggattctcctgcctcagtcttcagagtatttgggactacagacacacgccaccatgcctggctaattttttttttttgagacggagtctcgctctgttacccaggctggagtgcagtggcgtgatcttggctcactgcaacgtccgcctcccgggttcacgccattctcctgcctcagcctcccgagtagctgggactacaggagcccaccaccacgcctggctaattttttgtatttttactagagacggggtttcgccgtgttagccaggatggtctccatatcctgacctcgtgatccgcctgcctcggccttccaaagtgctaggattataggcgtgagccactgcgcctggccaatgcctggctaatttttttatatttttggtagagacagggttttgccatgttgcccaggctggtcttgaaatcctgacctcaggtgatccgcccgccttggcctcccaaagtgctgggattacaggcatgagccaccacgcccggccatgtactttatgttaaaatgggatcatattctagatcagcattatccagtagaaatttaaatttttaatacagggccaggcacggtggctcatgcctgtaatcccagcactttcggaggccgaggcgggtggatcgcaaggtcaggagatttgagatcatcctggctaacagatgggtaaaaacccatctctactaaaaatacaaaaaattagccatgcatggtggcatgcgcctgtagtcccagctactcgggaggctgaggccggagaatcacttgaacccgggaggcagaggttgcagtgagccgagatcgcgccactgcattccaacctgggtgacagagcgagactccgtctgaaaaaaaaaaaaaatttaacacgtatgtagacaatgtgcaaggcaccattccatgtgcatcgtatgtagtaactcttaattctcacgataaccctgaggtagatattattaccccgttctacaaaaggagaaacagtcctggggagacaggataagtcaccggccaaggcacacagccagctacatgtggcccccgcgtgacggctggtctctgtaggcgaggctttgtccagatgcgtgggtagaaggtctggcccggaaagaggaactgacagcaaggctaagccaatgtctgcccctgggggcagaaagtcacctcctgctctccctccactgtccacagaggtagctcagacagggtgggggtcacaggagaacgaagggagaagggggtagttcctgggcagcaaaatcaggtggtgaagggaggcatcagaggatggcaattagagaggccattagaggggaaccacaggcagacagggtgacaggagggactactgacacaaggtgaagagatggcccagccggacggggtggctcacatctgtaatcccagcattttgggagcccgaggtgggtggatcacttgaggtcaggagttcgaggccccaacatggcaaaaccccatctcttctaaaaatacaaaaattagccgggcatgatggcagatgcctgtaatccctgctactcgggaggctgaggcaggaaaattgcctgaatccaggaggtggaggttgcaatgagacgagatcatgacactgcactccaccctgggcaacagagcaagagactgactctgtctcataaaaaaaaagaaaaaagaaaaaaaaaaagagatggctgatggttaaagaggggttagcggtcaggggacacataagggtaaaggcaggaggcaagaggactggcagggggctgcccctgggccaccgggagcgacacaggatgagcatggagggaaagggagaaggggattctagggtcccagcctacccaagttgccctctggttccacctagcatgccagccagaggcccaggaaggaaccccgagccccccaccaaagctaaagatgagtcgctggagcctggtgaggggcaggatgaaggagctgctggagacagtggtgaacaggaccagagacgggtggcaatggttctggtgagggtgtgctgggctgggtggtgggaggggactcctgggtctgagggaggaggggctggggcctggacccctgagtctcagggaggaggaaagggtgggagtggggctgtgacccctaggtctgggaggagtggagggttagagctgagagcaggaactcctaggtcacagagaggagcggataaatggggcagagaacacctggggagagctggggcctccactgtgatgtcctctctcctgtaggagcccgagcaccttccggggcttcatgcagacctactatgacgaccacctgagggacctgggtccgctcaccaaggcctggttcctcgaatccaaagacagcctcttgaagaagacccacagcctgtgccccaggcttgtctgtggggacaaggaccagggttaaaatgttcataaaagccaggtgtggttgtggcgggtgcctgtagtcccagctactcaggaggctgaggtaggatgatggcttgagcccaggagttcgagaccagcctgggcaacacagcgagatctcttgggggtaaaacaaaaagaaaaaaaaaagttcatacttctccaataaataaagtctcacctgtgtccctgtctggatccttccccagtgtggccagaaaaaaacccaccccactgcctcccaggaatcaatgagtagaagaggtgacacctgatggggaaggaagagtagggaggtcgggaagggtatcaaggaataacaccctattgtgggcttgcggagaatgggggacttcaaggcgtgtcagtttcaggagggtgagggcaggagcgtgggtggagtcagcaggtccccatgatggccctcactgagagcttcgcccttgtctcctacaagctctgactccattcccagtgggca

SEQ ID NO: 10 - DNA, 7535 bp, Homo sapiens
cagcggatccttgatgctgccttaagtccggctcagaggggcgcagcgtggcctggggtcgctatcttcccatccggaacatctgccctgctgggggacactacgggccttcccttgcctgagggtagggtctcaaggtcacttgcccccagcttgacctggccggagtggctatagaggactttgtccctgcagactgcagcagcagagatgacactgtctctgagtgcagagatgggggcagggagctgggagagggttcaagctactggaacagcttcagaacaactagggtactaggaactgctgtgtcagggagaaggggctcaaggactcgcaggcctgggaggaggggcctaggccagccatgggagttgggtcacctgtgtctgaggacttggtgctgtctggattttgccaacctagggctggggtcagctgatgcccaccacgactcccgagcctccaggaactgaaaccctgtctgcccccagggtctggggaaggaggctgctgagtagaaccaaccccaggttaccaaccccacctcagccaccccttgccagccaaagcaaacaggcccggcccggcactgggggttccttctcgaaccaggagttcagcctcccctgacccgcagaatcttctgatcccacccgctccaggagccaggaatgagtcccagtctctcccagttctcactgtgtggttttgccattcgtcttgctgctgaaccacgggtttctcctctgaaacatctgggatttataacagggcttaggaaagtgacagcgtctgagcgttcactgtggcctgtccattgctagccctaacataggaccgctgtgtgccagggctgtcctccatgctcaatacacgttagcttgtcaccaaacatacccgtgccgctgctttcccagtctgatgagcaaaggaacttgatgctcagagaggacaagtcatttgcccaaggtcacacagctggcaactggcagagccaggattcacgccctggcaatttgactccagaatcctaaccttaacccagaagcacggcttcaagcccctggaaaccacaatacctgtggcagccagggggaggtgctggaatctcatttcacatgtggggagggggctcccctgtgctcaaggtcacaaccaaagaggaagctgtgattaaaacccaggtcccatttgcaaagcctcgacttttagcaggtgcatcatactgttcccacccctcccatcccacttctgtccagccgcctagccccactttcttttttttctttttttgagacagtctccctcttgctgaggctggagtgcagtggcgagatctcggctcactgtaacctccgcctcccgggttcaagcgattctcctgcctcagcctcccaagtagctaggattacaggcgcccgccaccacgcctggctaacttttgtatttttagtagagatggggtttcaccatgttggccaggctggtctcaaactcctgaccttaagtgattcgcccactgtggcctcccaaagtgctgggattacaggcgtgagctaccgcccccagcccctcccatcccacttctgtccagccccctagccctactttctttctgggatccaggagtccagatccccagccccctctccagattacattcatccaggcacaggaaaggacagggtcaggaaaggaggactctgggcggcagcctccacattccccttccacgcttggcccccagaatggaggagggtgtctggattactgggcgaggtgtcctcccttcctggggactgtggggggtggtcaaaagacctctatgccccacctccttcctccctctgccctgctgtgcctggggcagggggagaacagcccacctcgtgactgggggctggcccagcccgccctatccctgggggagggggcgggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccaggtagtctcaggagagctactcggggtcgggcttggggagaggaggagcgggggtgaggcaagcagcaggggactggacctgggaagggctgggcagcagagacgacccgacccgctagaaggtggggtggggagagcagctggactgggatgtaagccatagcaggactccacgagttgtcactatcatttatcgagcacctactgggtgtccccagtgtcctcagatctccataactggggagccaggggcagcgacacggtagctagccgtcgattggagaactttaaaatgaggactgaattagctcataaatggaacacggcgcttaactgtgaggttggagcttagaatgtgaagggagaatgaggaatgcgagactgggactgagatggaaccggcggtggggagggggtggggggatggaatttgaaccccgggagaggaagatggaattttctatggaggccgacctggggatggggagataagagaagaccaggagggagttaaatagggaatgggttgggggcggcttggtaaatgtgctgggattaggctgttgcagataatgcaacaaggcttggaaggctaacctggggtgaggccgggttggggccgggctgggggtgggaggagtcctcactggcggttgattgacagtttctccttccccagactggccaatcacaggcaggaagatgaaggttctgtgggctgcgttgctggtcacattcctggcaggtatgggggcggggcttgctcggttccccccgctcctccccctctcatcctcacctcaacctcctggccccattcaggcagaccctgggccccctcttctgaggcttctgtgctgcttcctggctctgaacagcgatttgacgctctctgggcctcggtttcccccatccttgagataggagttagaagttgttttgttgttgttgtttgttgttgttgttttgtttttttgagatgaagtctcgctctgtcgcccaggctggagtgcagtggcgggatctcggctcactgcaagctccgcctcccaggtccacgccattctcctgcctcagcctcccaagtagctgggactacaggcacatgccaccacacccgactaacttttttgtattttcagtagagacggggtttcaccatgttggccaggctggtctggaactcctgacctcaggtgatctgcccgtttcgatctcccaaagtgctgggattacaggcgtgagccaccgcacctggctgggagttagaggtttctaatgcattgcaggcagatagtgaataccagacacggggcagctgtgatctttattctccatcacccccacacagccctgcctggggcacacaaggacactcaatacatgcttttccgctgggcgcggtggctcacccctgtaatcccagcactttgggaggccaaggtgggaggatcacttgagcccaggagttcaacaccagcctgggcaacatagtgagaccctgtctctactaaaaatacaaaaattagccaggcatggtgccacacacctgtgctctcagctactcaggaggctgaggcaggaggatcgcttgagcccagaaggtcaaggttgcagtgaaccatgttcaggccgctgcactccagcctgggtgacagagcaagaccctgtttataaatacataatgctttccaagtgattaaaccgactcccccctcaccctgcccaccatggctccaaagaagcatttgtggagcaccttctgtgtgcccctaggtactagatgcctggacggggtcagaaggaccctgacccaccttgaacttgttccacacaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgaggtgagtgtccccatcctggcccttgaccctcctggtgggcggctatacctccccaggtccaggtttcattctgcccctgtcgctaagtcttggggggcctgggtctctgctggttctagcttcctcttcccatttctgactcctggctttagctctctggaattctctctctcagctttgtctctctctcttcccttctgactcagtctctcacactcgtcctggctctgtctctgtccttccctagctcttttatatagagacagagagatggggtctcactgtgttgcccaggctggtcttgaacttctgggctcaagcgatcctcccgcctcggcctcccaaagtgctgggattagaggcatgagccaccttgcccggcctcctagctccttcttcgtctctgcctctgccctctgcatctgctctctgcatctgtctctgtctccttctctcggcctctgccccgttccttctctccctcttgggtctctctggctcatccccatctcgcccgccccatcccagcccttctccccgcctcccactgtgcgacaccctcccgccctctcggccgcagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatctgctggcctccccctgtgatttcctctaagccccagcctcagtttctctttctgcccacatactggccacacaattctcagccccctcctctccatctgtgtctgtgtgtatctttctctctgcccttttttttttttttagacggagtctggctctgtcacccaggctagagtgcagtggcacgatcttggctcactgcaacctctgcctcttgggttcaagcgattctgctgcctcagtagctgggattacaggctcacaccaccacacccggctaatttttgtatttttagtagagacgagctttcaccatgttggccaggcaggtctcaaactcctgaccaagtgatccacccgccggcctcccaaagtgctgagattacaggcctgagccaccatgcccggcctctgcccctctttcttttttagggggcagggaaaggtctcaccctgtcacccgccatcacagctcactgcagcctccacctcctggactcaagtgataagtgatcctcccgcctcagcctttccagtagctgagactacaggcgcataccactaggattaatttgggggggggggtggtgtgtgtggagatggggtctggctttgttggccaggctgatgtggaattcctgggctcaagcgatactcccaccttggcctcctgagtagctgagactactggctagcaccaccacacccagctttttattattatttgtagagacaaggtctcaatatgttgcccaggctagtctcaaacccctgggctcaagagatcctccgccatcggcctcccaaagtgctgggattccaggcatggggctccgagcccggcctgcccaacttaataatacttgttcctcagagttgcaactccaaatgacctgagattggtgcctttattctaagctattttcattttttttctgctgtcattattctcccccttctctcctccagtcttatctgatatctgcctccttcccacccaccctgcaccccatcccacccctctgtctctccctgttctcctcaggagactctggcttcctgttttcctccacttctatcttttatctctccctcctacggtttcttttctttctccccggcctgcttgtttctcccccaacccccttcatctggatttcttcttctgccattcagtttggtttgagctctctgcttctccggttccctctgagctagctgtcccttcacccactgtgaactgggtttccctgcccaaccctcattctctttctttctttctttttttttttttttttttttttttttttttgagacagagtcttgctctgttgcccagcctggagtgcagtggtgcaatcttggttcactgcaacctccacttcccagattcaagcaattctcctgcctcagcctccagagtagctgggattacaggcgtgtcccaccacacccgactaatttttgtatttttggtagagacaaggcttcggcattgttggccaggcaggtctcgaactcctgacctcaagtaatctgcctgcctcaccctcccaaagtgctgggattacaggcatgagccacctcacccggaccatccctcattctccatcctttcctccagttgtgatgtctacccctcatgtttcccaacaagcctactgggtgctgaatccaggctgggaagagaagggagcggctcttctgtcggagtctgcaccaggcccatgctgagacgagagctggcgctcagagaggggaagcttggatggaagcccaggagccgccggcactctcttcccctcccaccccctcagttctcagagacggggaggagggttcccaccaacgggggacaggctgagacttgagcttgtatctcctgggccagctgcaacatctgcttgtccctctgcccatcttggctcctgcacaccctgaacttggtgctt